(19) United States
(12) Patent Application Publication
(10) Pub. No.: US 2024/0075142 A1
(43) Pub. Date: Mar. 7, 2024

(54) Compositions and Methods for Treating Cancer with Fully Human Anti-CD20/CD19 Immunotherapy

(71) Applicant: LENTIGEN TECHNOLOGY, INC., Gaithersburg, MD (US)

(72) Inventors: Dina Schneider, Potomac, MD (US); Peirong Hu, Ijamsville, MD (US)

(21) Appl. No.: 18/455,675

(22) Filed: Aug. 25, 2023

Related U.S. Application Data
(60) Provisional application No. 63/401,288, filed on Aug. 26, 2022.

(51) Int. Cl. A61K 39/00; A61P 35/00; C07K 16/28; C12N 5/0783; C12N 15/63

(52) U.S. Cl. CPC A61K 39/4631; A61K 39/4611; A61P 35/00; C07K 16/2803; C07K 16/2887; C12N 5/0636; C12N 15/63; A61K 2239/21; A61K 2239/22

(57) ABSTRACT

Chimeric antigen receptors containing fully human CD20/CD19 antigen binding domains are disclosed. Nucleic acids, recombinant expression vectors, host cells, antigen binding fragments, and pharmaceutical compositions, relating to the chimeric antigen receptors are also disclosed. Methods of treating or preventing cancer in a subject, and methods of making chimeric antigen receptor T cells are also disclosed.

### CROSS-REFERENCE TO RELATED APPLICATIONS

This application claims priority to U.S. Provisional Patent Application No. 63/401,288, filed on Aug. 26, 2022, the entire contents of which are incorporated herein by reference in its entirety.

### SEQUENCE LISTING

This application contains a Sequence Listing that has been submitted electronically as an XML file named 42449-0089001.xml. The XML file, created on Aug. 17, 2023, is 74,928 bytes in size.

The material in the XML file is hereby incorporated by reference in its entirety.

### FIELD OF THE DISCLOSURE

This application relates to the field of cancer, particularly to fully human CD20 and fully human CD19 antigen binding domains (hereinafter termed “fully human CD20/CD19”) and chimeric antigen receptors (CARs) containing such fully human CD20 and fully human CD19 antigen binding domains and methods of use thereof.

### BACKGROUND

Cancer is one of the most deadly threats to human health. In the U.S. alone, cancer affects nearly 1.3 million new patients each year, and is the second leading cause of death after cardiovascular disease, accounting for approximately 1 in 4 deaths. Solid tumors are responsible for most of those deaths. Although there have been significant advances in the medical treatment of certain cancers, the overall 5-year survival rate for all cancers has improved only by about 10% in the past 20 years. Cancers, or malignant tumors, metastasize and grow rapidly in an uncontrolled manner, making treatment extremely difficult.

CD19 is a 85-95 kDa transmembrane cell surface glycoprotein receptor. CD19 is a member of immunoglobulin (Ig) superfamily of proteins, and contains two extracellular Ig-like domains, a transmembrane, and an intracellular signaling domain (Tedder T F, Isaacs, C M, 1989, J Immunol 143:712-171). CD19 modifies B cell receptor signaling, lowering the triggering threshold for the B cell receptor for antigen (Carter, R H, and Fearon, D T, 1992, Science, 256:105-107), and co-ordinates with CD81 and CD21 to regulate this essential B cell signaling complex (Bradbury, L E, Kansas G S, Levy S, Evans R L, Tedder T F, 1992, J Immunol, 149:2841-50). During B cell ontogeny CD19 is able to signal at the pro-B, pre-pre-B cell, pre-B, early B cell stages independent of antigen receptor, and is associated with Src family protein tyrosine kinases, is tyrosine phosphorylated, inducing both intracellular calcium mobilization and inositol phospholipid signaling (Uckun F M, Burkhardt A L, Jarvis L, Jun X, Stealy B, Dibirdik I, Myers D E, Tuel-Ahlgren L, Bolen J B, 1983, J Biol Chem 268:21172-84). The key point of relevance for treatment of B cell malignancies is that CD19 is expressed in a tightly regulated manner on normal B cells, being restricted to early B cell precursors at the stage of IgH gene rearrangement, mature B cells, but not expressed on hematopoietic stem cells, or mature plasma cells (Anderson, K C, Bates, M P, Slaughenhout B L, Pinkus G S, Schlossman S F, Nadler L M, 1984, Blood 63:1424-1433).

CD20 (also termed LEU-16, MS4A1) is a membrane-spanning 4A family protein that is expressed on the surface of B cells from pro-B phase to mature B cell phase, and plays a role in B cell development and differentiation. CD20 antigen is also expressed on a variety of hematological tumors, and a variety of monoclonal anti-CD20 antibodies have been applied over the years for the treatment of CD20-positive malignancies (Reviewed in Lim, Sean H. et al. “Anti-CD20 Monoclonal Antibodies: Historical and Future Perspectives.” Haematologica 95.1 (2010): 135-143. PMC. Web. 31 Jul. 2017.) The anti CD20 monoclonal antibody Rituximab (Rituxan (@) is widely used in treatment of B-cell lymphomas, such as follicular lymphoma (FL), and diffuse large B cell lymphoma (DLBCL), and chronic lymphocytic leukemia (CLL) (Rituxan prescribing information).

The traditional treatment approaches for B-lineage leukemias and lymphomas may involve chemotherapy, radiotherapy and stem cells transplant (see the world wide web at mayclinic.org). High toxicity associated with these treatments, as well as the risk of complications, such as relapse, secondary malignancy, or GVHD, motivate the search for better therapeutic alternatives. The expression of CD19 on both adult and pediatric (pre-B-ALL) B cell malignancies has led to exploiting this target for both antibody and chimeric antigen receptor (CAR)-T cell-based therapy (Kochenderfer J N, Wilson W H, Janik J E, Dudley M E, Stetler-Stevenson M, Feldman S A, Maric I, Raffeld M, Nathan D A, Lanier B J, Morgan R A, Rosenberg S A, 2010, Blood 116:4099-102; Lee D W, Kochenderfer J N, Stetler-Stevenson M, Cui Y K, Delbrook C, Feldman S A, Orentas R, Sabatino M, Shah N N, Steinberg S M, Stroncek D, Tschernia N, Yuan C, Zhang H, Zhang L, Rosenberg S A, Wayne A S, Mackall C L, 2015, Lancet 385:517-28). Moreover, the presence of CD20 antigen on lymphomas (DLBCL, FL), and leukemias (CLL) make it an attractive additional target for efficient tumor elimination and for the prevention of tumor antigen escape.

The present standard of care for B-lineage leukemias may consists of remission induction treatment by high dose of chemotherapy or radiation, followed by consolidation, and may feature stem cell transplantation and additional courses of chemotherapy as needed (see the world wide web at cancer.gov). High toxicity associated with these treatments, as well as the risk of complications, such as relapse, secondary malignancy, or GVHD, motivate the search for better therapeutic alternatives. The expression of CD19 on both adult and pediatric (pre-B-ALL) B cell malignancies has led to exploiting this target for both antibody and chimeric antigen receptor (CAR)-T cell-based therapy (Kochenderfer J N, Wilson W H, Janik J E, Dudley M E, Stetler-Stevenson M, Feldman S A, Maric I, Raffeld M, Nathan D A, Lanier B J, Morgan R A, Rosenberg S A, 2010, Blood 116:4099-102; Lee D W, Kochenderfer J N, Stetler-Stevenson M, Cui Y K, Delbrook C, Feldman S A, Orentas R, Sabatino M, Shah N N, Steinberg S M, Stroncek D, Tschernia N, Yuan C, Zhang H, Zhang L, Rosenberg S A, Wayne A S, Mackall C L, 2015, Lancet 385:517-28).

A number of novel approaches to treat B cell leukemia and lymphoma have been developed, including bi-specific antibodies that link an anti-CD19 or anti-CD20 binding motif to a T cell binding motif (i.e. Blinatumomab, Blincyto® indicated for the treatment of Philadelphia chromosome-negative relapsed or refractory B-cell precursor acute lymphoblastic leukemia (ALL). To date, many of the binding moieties for CD19 or CD20 employed in CAR constructs utilize a domain derived from murine antibodies. A number of these products are currently being considered for approval including those developed by Novartis and Kite Pharmaceuticals. In April of 2017 Novartis announced that CTL019 (tisagenlecleucel) received FDA breakthrough designation for treatment of adult patients with refractory or recurrent (r/r) DLBCL (diffuse large B cell lymphoma) who failed two or more prior therapies, adding this designation to that for r/r B-cell acute lymphoblastic leukemia (ALL). These indications were based on the Phase II JULIET study (NCT02445248) and the ELIANA study (NCT02435849), respectively. The JULIET trial showed and overall response rate (ORR) of 45%, with a 37% complete response (CR), and an 8% partial response (PR) at three months. In the ELIANA study, 82% of patients infused with the product achieved CR or CR with incomplete count recovery, and the relapse free survival rate at 6 months was 60%. The CAR-T product from Kite Pharmaceuticals (KTE-C19, axicabtagene ciloleucel) was granted breakthrough designation for diffuse large B-cell lymphoma (DLBLC), transformed follicular lymphoma (TFL), and primary mediastinal B-cell lymphoma (PMBCL). In the Kite ZUMA-3 phase II trial of KTE-C19 in r/r ALL, a 73% CR was reported (at 2 months or greater). Whether antibody of CAR-T therapies are utilized, there are still a significant number of patients who are not helped by these therapies, and there is considerable room for improved therapeutic approaches.

Chimeric Antigen Receptors (CARs) are hybrid molecules comprising three essential units: (1) an extracellular antigen-binding motif, (2) linking/transmembrane motifs, and (3) intracellular T-cell signaling motifs (Long A H, Haso W M, Orentas R J. Lessons learned from a highly-active CD22-specific chimeric antigen receptor. Oncoimmunology. 2013; 2 (4):e23621). The antigen-binding motif of a CAR is commonly fashioned after an single chain Fragment variable (ScFv), the minimal binding domain of an immunoglobulin (Ig) molecule. Alternate antigen-binding motifs, such as receptor ligands (i.e., IL-13 has been engineered to bind tumor expressed IL-13 receptor), intact immune receptors, library-derived peptides, and innate immune system effector molecules (such as NKG2D) also have been engineered. Alternate cell targets for CAR expression (such as NK or gamma-delta T cells) are also under development (Brown C E et al Clin Cancer Res. 2012; 18(8):2199-209; Lehner M et al. PLoS One. 2012; 7 (2):e31210). There remains significant work to be done with regard to defining the most active T-cell population to transduce with CAR vectors, determining the optimal culture and expansion techniques, and defining the molecular details of the CAR protein structure itself.

The linking motifs of a CAR can be a relatively stable structural domain, such as the constant domain of IgG, or designed to be an extended flexible linker. Structural motifs, such as those derived from IgG constant domains, can be used to extend the ScFv binding domain away from the T-cell plasma membrane surface. This may be important for some tumor targets where the binding domain is particularly close to the tumor cell surface membrane (such as for the disialoganglioside GD2; Orentas et al., unpublished observations). To date, the signaling motifs used in CARs always include the CD3-ζ chain because this core motif is the key signal for T cell activation. The first reported second-generation CARs featured CD28 signaling domains and the CD28 transmembrane sequence. This motif was used in third-generation CARs containing CD137 (4-1BB) signaling motifs as well (Zhao Y et al J Immunol. 2009; 183 (9): 5563-74). With the advent of new technology, the activation of T cells with beads linked to anti-CD3 and anti-CD28 antibody, and the presence of the canonical “signal 2” from CD28 was no longer required to be encoded by the CAR itself. Using bead activation, third-generation vectors were found to be not superior to second-generation vectors in in vitro assays, and they provided no clear benefit over second-generation vectors in mouse models of leukemia (Haso W, Lee D W, Shah N N, Stetler-Stevenson M, Yuan C M, Pastan I H, Dimitrov D S, Morgan R A, FitzGerald D J, Barrett D M, Wayne A S, Mackall C L, Orentas R J. Anti-CD22-chimeric antigen receptors targeting B cell precursor acute lymphoblastic leukemia, Blood. 2013; 121 (7):1165-74; Kochenderfer J N et al. Blood. 2012; 119 (12):2709-20). This is borne out by the clinical success of CD19-specific CARs that are in a second generation CD28/CD3-ζ (Lee D W et al. American Society of Hematology Annual Meeting. New Orleans, L A; Dec. 7-10, 2013) and a CD137/CD3-ζ signaling format (Porter D L et al. N Engl J Med. 2011; 365 (8): 725-33). In addition to CD137, other tumor necrosis factor receptor superfamily members such as OX40 also are able to provide important persistence signals in CAR-transduced T cells (Yvon E et al. Clin Cancer Res. 2009; 15(18):5852-60). Equally important are the culture conditions under which the CAR T-cell populations were cultured, for example the inclusion of the cytokines IL-2, IL-7, and/or IL-15 (Kaiser A D et al. Cancer Gene Ther. 2015; 22(2):72-78).

Current challenges in the more widespread and effective adaptation of CAR therapy for cancer relate to a paucity of compelling targets. Creating binders to cell surface antigens is now readily achievable, but discovering a cell surface antigen that is specific for tumor while sparing normal tissues remains a formidable challenge. One potential way to imbue greater target cell specificity to CAR-expressing T cells is to use combinatorial CAR approaches. In one system, the CD3-ζ and CD28 signal units are split between two different CAR constructs expressed in the same cell; in another, two CARs are expressed in the same T cell, but one has a lower affinity and thus requires the alternate CAR to be engaged first for full activity of the second (Lanitis E et al. Cancer Immunol Res. 2013; 1(1):43-53; Kloss C C et al. Nat Biotechnol. 2013; 31(1):71-5). A second challenge for the generation of a single ScFv-based CAR as an immunotherapeutic agent is tumor cell heterogeneity. At least one group has developed a CAR strategy for glioblastoma whereby the effector cell population targets multiple antigens (HER2, IL-13Ra, EphA2) at the same time in the hope of avoiding the outgrowth of target antigen-negative populations. (Hegde M et al. Mol Ther. 2013; 21(11):2087-101).

T-cell-based immunotherapy has become a new frontier in synthetic biology; multiple promoters and gene products are envisioned to steer these highly potent cells to the tumor microenvironment, where T cells can both evade negative regulatory signals and mediate effective tumor killing. The elimination of unwanted T cells through the drug-induced dimerization of inducible caspase 9 constructs with chemical-based dimerizers, such as AP1903, demonstrates one way in which a powerful switch that can control T-cell populations can be initiated pharmacologically (Di Stasi A et al. N Engl J Med. 2011; 365(18):1673-83). The creation of effector T-cell populations that are immune to the negative regulatory effects of transforming growth factor-p by the expression of a decoy receptor further demonstrates the degree to which effector T cells can be engineered for optimal antitumor activity (Foster A E et al. J Immunother. 2008; 31(5):500-5). Thus, while it appears that CARs can trigger T-cell activation in a manner similar to an endogenous T-cell receptor, a major impediment to the clinical application of this technology to date has been limited in vivo expansion of CAR+ T cells, rapid disappearance of the cells after infusion, and disappointing clinical activity. This may be due in part to the murine origin of some of the CAR sequences employed.

The use of Blinotumomab (bi-specific anti-CD19 and anti-CD3 antibody) has shown impressive results for the gravely ill patients who have received this therapy. Nevertheless the durable remission rate is less than 40%, and at best only 50% of responders can be salvaged to hematopoietic stem cell transplant (HSCT) (see Gore et al., 2014, NCT01471782 and Von Stackelberg, et al., 2014, NCT01471782, summarized in: Benjamin, J E, Stein A S, 2016, Therapeutic Advances in Hematology 7:142-156). The requirement of patients who have received either bi-specific antibody or CAR-T therapy to subsequently undergo HSCT in order to maintain durable responses remains an area of active debate. Although high responses are reported for CD19 CAR-T trials, some even greater than 90%, if the trials are re-cast as “intent to treat” trials the number may be closer to 70% (Davis K L, Mackall C L, 2016, Blood Advances 1:265-268). The best results at 12 months post-CAR19 treatment reported show a RFS of 55% and OS of 79% in patients who were able to receive the T cell product at the University of Pennsylvania (Maude S L, Teachey D T, Rheingold S R, Shaw P A, Aplenc R, Barrett D M, Barker C S, Callahan C, Frey N V, Farzana N, Lacey S F, Zheng A, Levine B, Melenhorst J J, Motley L, Prter D L, June C H, Grupp S A, 2016, J Clin Oncol 34, no15_suppl (May 2016) 3011-3011).

Accordingly, there is an urgent and long felt need in the art for discovering novel compositions and methods for treatment of B-ALL and other CD19 and/or CD20-expressing B cell malignancies using an approach that can exhibit specific and efficacious anti-tumor effect without the aforementioned short comings.

The present invention addresses these needs by providing CAR compositions and therapeutic methods that can be used to treat cancers and other diseases and/or conditions. In particular, the present invention as disclosed and described herein provides CARs that may be used for the treatment of diseases, disorders or conditions associated with dysregulated expression of CD20 and/or CD19 and which CARs contain tandem fully human CD20/CD19 antigen binding domains that exhibit one or more of the following properties: i) a high surface expression on transduced T cells; ii) a high degree of cytolysis of CD20 and/or CD19-expressing cells, in which the transduced T cells demonstrate in vivo expansion and persistence; iii) multi-targeting to overcome antigen escape; iv) armor so as to overcome immunosuppression in tumor microenvironment (TME); v) cytokine stimulated element to promote autonomous T cell stimulation with cytokines, resulting in heightened anti-tumor cytotoxicity, expansion, memory formation, cytokine secretion, persistence; vi) digestive enzymes to overcome the physical barrier of tumor stroma/extracellular matrix (ECM) and enable CAR T tumor penetration; and vii) an on-switch or off-switch, to control the expression of the CAR, or the co-expressed functional “booster” element(s), as well as therapeutic methods of using such CARs that can be used to treat solid tumors, including tumors expressing a targetable antigen, such as ROR1, Mesothelin, HER2/ERBB2, Folate Receptor I, PSMA, CD276, Claudin 6, Claudin 1.28, MUC1, MUC16, GD2, LRRC15, GPC3, FGFR4, FAP, IL-13Ra, EphA2, or other antigens, or any combination thereof, as well as other diseases and/or conditions expressing CAR-relevant targets, including, for example and not by way of limitation, autoimmune diseases, alloimmune diseases, and autoaggressive diseases comprising rheumatoid arthritis, lupus, celiac disease, Sjögren's syndrome, multiple sclerosis, polymyalgia rheumatica, ankylosing spondylitis, type 1 diabetes, alopecia areata, vasculitis, temporal arteritis, post-streptococcal autoimmune disorder, antineuronal antibody-mediated neuropsychiatric disorder, immune-mediated extrapyramidal movement disorder, Sydenham chorea, alloimmune hemolytic disease, pulmonary fibrosis, systemic scleroderma, or fibrotic disease.

### SUMMARY OF THE INVENTION

Novel tandem fully human CD20 and CD19-targeting antibodies or antigen binding domains thereof in which the CD19 targeting moiety is positioned either before or after the CD20 targeting moiety in the amino acid sequence (hereinafter termed “fully human CD20/CD19”), and chimeric antigen receptors (tandem CARs) that contain such CD20 and/or CD19 antigen binding domains are provided herein, as well as host cells (e.g., T cells) expressing the receptors, and nucleic acid molecules encoding the receptors. The CARs exhibit a high surface expression on transduced T cells, with a high degree of cytolysis, and with transduced T cell expansion and persistence in vivo. Methods of using the disclosed CARs, host cells, and nucleic acid molecules are also provided, for example, to treat a cancer in a subject.

In one aspect, an isolated nucleic acid molecule encoding a tandem fully human CD20/CD19 chimeric antigen receptor (CAR) is provided comprising, from N-terminus to C-terminus, at least one fully human CD20/CD19 antigen binding domain, at least one transmembrane domain, and at least one intracellular signaling domain, wherein the tandem fully human CD20/CD19 CAR comprises a nucleic acid sequence selected from the group consisting of SEQ ID NOs: 1, 3, and 48.

In one aspect, an isolated nucleic acid molecule encoding a tandem fully human CD20/CD19 chimeric antigen receptor (CAR) is provided comprising, from N-terminus to C-terminus, at least one fully human CD20/CD19 antigen binding domain, at least one transmembrane domain, and at least one intracellular signaling domain, wherein the tandem fully human CD20/CD19 CAR encoded by the nucleic acid sequence selected from the group consisting of SEQ ID NOs: 1, 3, and 48 encodes a tandem fully human CD20/CD19 CAR comprising the amino acid sequence selected from the group consisting of SEQ ID NO. 2 4, and 49.

In one embodiment, an isolated nucleic acid molecule encoding the CAR is provided wherein the encoded extracellular fully human CD20/CD19 antigen binding domain comprises at least one single chain variable fragment of an antibody that binds to fully human CD20/CD19.

In another embodiment, an isolated nucleic acid molecule encoding the CAR is provided wherein the encoded extracellular fully human CD20/CD19 antigen binding domain comprises at least one heavy chain variable region of an antibody that binds to fully human CD20/CD19.

In yet another embodiment, an isolated nucleic acid molecule encoding the CAR is provided wherein the encoded CAR extracellular fully human CD20/CD19 antigen binding domain further comprises at least one lipocalin-based antigen binding antigen (anticalins) that binds to fully human CD20/CD19.

In one embodiment, an isolated nucleic acid molecule is provided wherein the encoded extracellular fully human CD20/CD19 antigen binding domain is connected to the transmembrane domain by a linker domain.

In another embodiment, an isolated nucleic acid molecule encoding the CAR is provided wherein the encoded fully human CD20/CD19 extracellular antigen binding domain is preceded by a sequence encoding a leader or signal peptide.

In yet another embodiment, an isolated nucleic acid molecule encoding the CAR is provided comprising at least one fully human CD20/CD19 antigen binding domain encoded by a nucleotide sequence comprising a fully human CD20/CD19 nucleotide sequence contained within SEQ ID Nos: 1 and 3, respectively, and wherein the CAR additionally encodes an extracellular antigen binding domain targets an antigen that includes, but is not limited to, CD22, ROR1, mesothelin, CD33, CD38, CD123 (IL3RA), CD138, BCMA (CD269), GPC2, GPC3, FGFR4, c-Met, PSMA, Glycolipid F77, EGFRvIII, GD-2, TSLPR, NY-ESO-1 TCR, MAGE A3 TCR, or any combination thereof.

In yet another embodiment, an isolated nucleic acid molecule encoding the CAR is provided comprising at least one fully human CD20/CD19 antigen binding domain encoded by a nucleotide sequence comprising a fully human CD20/CD19 nucleotide sequence contained within SEQ ID No: 48, and wherein the CAR additionally encodes an extracellular antigen binding domain targets an antigen that includes, but is not limited to, CD22, ROR1, mesothelin, CD33, CD38, CD123 (IL3RA), CD138, BCMA (CD269), GPC2, GPC3, FGFR4, c-Met, PSMA, Glycolipid F77, EGFRvIII, GD-2, TSLPR, NY-ESO-1 TCR, MAGE A3 TCR, or any combination thereof.

In certain embodiments, an isolated nucleic acid molecule encoding the CAR is provided wherein the additionally encoded extracellular antigen binding domain comprises an anti-CD22 ScFv antigen binding domain, an anti-ROR1 ScFv antigen binding domain, an anti-mesothelin ScFv antigen binding domain, an anti-CD33 ScFv antigen binding domain, an anti-CD38 ScFv antigen binding domain, an anti-CD123 (IL3RA) ScFv antigen binding domain, an anti-CD138 ScFv antigen binding domain, an anti-BCMA (CD269) ScFv antigen binding domain, an anti-GPC2 ScFv antigen binding domain, an anti-GPC3 ScFv antigen binding domain, an anti-FGFR4 ScFv antigen binding domain, an anti-TSLPR ScFv antigen binding domain an anti-c-Met ScFv antigen binding domain, an anti-PMSA ScFv antigen binding domain, an anti-glycolipid F77 ScFv antigen binding domain, an anti-EGFRvIII ScFv antigen binding domain, an anti-GD-2 ScFv antigen binding domain, an anti-NY-ESO-1 TCR ScFv antigen binding domain, an anti-MAGE A3 TCR ScFv antigen binding domain, or an amino acid sequence with 85%, 90%, 95%, 96%, 97%, 98% or 99% identity thereof, or any combination thereof.

In one aspect, the CARs provided herein further comprise a linker or spacer domain.

In one embodiment, an isolated nucleic acid molecule encoding the CAR is provided wherein the extracellular fully human CD20/CD19 antigen binding domain, the intracellular signaling domain, or both are connected to the transmembrane domain by a linker or spacer domain.

In one embodiment, an isolated nucleic acid molecule encoding the CAR is provided wherein the encoded linker domain is derived from the extracellular domain of CD8 or CD28, and is linked to a transmembrane domain.

In another embodiment, an isolated nucleic acid molecule encoding the CAR is provided wherein the encoded CAR further comprises a transmembrane domain that comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD83, CD86, CD134, CD137 and CD154, or a combination thereof.

In yet another embodiment, an isolated nucleic acid molecule encoding the CAR is provided wherein the encoded intracellular signaling domain further comprises a CD3 zeta intracellular domain.

In one embodiment, an isolated nucleic acid molecule encoding the CAR is provided wherein the encoded intracellular signaling domain is arranged on a C-terminal side relative to the CD3 zeta intracellular domain.

In another embodiment, an isolated nucleic acid molecule encoding the CAR is provided wherein the encoded at least one intracellular signaling domain comprises a costimulatory domain, a primary signaling domain, or a combination thereof.

In further embodiments, an isolated nucleic acid molecule encoding the CAR is provided wherein the encoded at least one costimulatory domain comprises a functional signaling domain of OX40, CD70, CD27, CD28, CD5, ICAM-1, LFA-1 (CD11a/CD18), ICOS (CD278), DAP10, DAP12, and 4-1BB (CD137), or a combination thereof.

In one embodiment, an isolated nucleic acid molecule encoding the CAR is provided that further contains a leader sequence or signal peptide wherein the leader or signal peptide nucleotide sequence comprises the nucleotide sequence of SEQ ID NO: 11.

In yet another embodiment, an isolated nucleic acid molecule encoding the CAR is provided wherein the encoded leader sequence comprises the amino acid sequence of SEQ ID NO: 12.

In one aspect, a chimeric antigen receptor (CAR) is provided herein comprising, from N-terminus to C-terminus, at least one fully human CD20/CD19 antigen binding domain, at least one transmembrane domain, and at least one intracellular signaling domain.

In one embodiment, a CAR is provided wherein the extracellular fully human CD20/CD19 antigen binding domain comprises at least one single chain variable fragment of an antibody that binds to the antigen, or at least one heavy chain variable region of an antibody that binds to the antigen, or a combination thereof.

In another embodiment, a CAR is provided wherein the at least one transmembrane domain comprises a transmembrane domain of a protein selected from the group consisting of the alpha, beta or zeta chain of the T-cell receptor, CD28, CD3 epsilon, CD45, CD4, CD5, CD8, CD9, CD16, CD22, CD33, CD37, CD64, CD80, CD86, CD134, CD137, CD154, TNFRSF19, or a combination thereof.

In some embodiments, the CAR is provided wherein CAR additionally encodes an extracellular antigen binding domain comprising CD22, ROR1, mesothelin, CD33, CD38, CD123 (IL3RA), CD138, BCMA (CD269), GPC2, GPC3, FGFR4, TSLPR, c-Met, PSMA, Glycolipid F77, EGFRvIII, GD-2, TSLPR, NY-ESO-1 TCR, MAGE A3 TCR, or an amino acid sequence with 85%, 90%, 95%, 96%, 97%, 98% or 99% identity thereof, or any combination thereof.

In one embodiment, the CAR is provided wherein the extracellular antigen binding domain comprises an anti-CD22 ScFv antigen binding domain, an anti-ROR1 ScFv antigen binding domain, an anti-mesothelin ScFv antigen binding domain, an anti-CD33 ScFv antigen binding domain, an anti-CD38 ScFv antigen binding domain, an anti-CD123 (IL3RA) ScFv antigen binding domain, an anti-CD138 ScFv antigen binding domain, an anti-BCMA (CD269) ScFv antigen binding domain, an anti-GPC2 ScFv antigen binding domain, an anti-GPC3 ScFv antigen binding domain, an anti-FGFR4 ScFv antigen binding domain, anti-TSLPR ScFv antigen binding domain, an anti-c-Met ScFv antigen binding domain, an anti-PMSA ScFv antigen binding domain, an anti-glycolipid F77 ScFv antigen binding domain, an anti-EGFRvIII ScFv antigen binding domain, an anti-GD-2 ScFv antigen binding domain, an anti-NY-ESO-1 TCR ScFv antigen binding domain, an anti-MAGE A3 TCR ScFv antigen binding domain, or an amino acid sequence with 85%, 90%, 95%, 96%, 97%, 98% or 99% identity thereof, or any combination thereof.

In another embodiment, a CAR is provided wherein the at least one intracellular signaling domain comprises a costimulatory domain and a primary signaling domain.

In yet another embodiment, a CAR is provided wherein the at least one intracellular signaling domain comprises a costimulatory domain comprising a functional signaling domain of a protein selected from the group consisting of OX40, CD70, CD27, CD28, CD5, ICAM-1, LFA-1 (CD11a/CD18), ICOS (CD278), DAP10, DAP12, and 4-1BB (CD137), or a combination thereof.

In one embodiment, the nucleic acid sequence encoding a CAR comprises the nucleic acid sequence of SEQ ID NO: 5, nucleotide sequence of CAR D0144 (CD20_CD19 CD8 BBz).

In one embodiment, the nucleic acid sequence encodes a CAR comprising the amino acid sequence of SEQ ID NO: 6 CAR D0144 (CD20_CD19 CD8 BBz).

In one embodiment, the nucleic acid sequence encoding a CAR comprises the nucleic acid sequence of SEQ ID NO: 7, nucleotide sequence of CAR D0255 (CD20_CD19 CD28) CD28 BBz.

In one embodiment, the nucleic acid sequence encodes a CAR comprising the amino acid sequence of SEQ ID NO: 8 CAR D0255 (CD20_CD19 CD28) CD28 BBz.

In another embodiment, the nucleic acid sequence encoding a CAR comprises the nucleic acid sequence of SEQ ID NO: 15, nucleotide sequence of CAR D0258 (CD20 CD8 BBz_CD19 CD8 CD28z).

In one embodiment, the nucleic acid sequence encodes a CAR comprising the amino acid sequence of SEQ ID NO: 16, CAR D0258 (CD20 CD8 BBz_CD19 CD8 CD28z).

In one aspect, the CARs disclosed herein are modified to express or contain a detectable marker for use in diagnosis, monitoring, and/or predicting the treatment outcome such as progression free survival of cancer patients or for monitoring the progress of such treatment.

In one embodiment, the nucleic acid molecule encoding the disclosed CARs can be contained in a vector, such as a viral vector. The vector is a DNA vector, an RNA vector, a plasmid vector, a cosmid vector, a herpes virus vector, a measles virus vector, a lentivirus vector, adenoviral vector, or a retrovirus vector, or a combination thereof.

In certain embodiments, the vector further comprises a promoter wherein the promoter is an inducible promoter, a tissue specific promoter, a constitutive promoter, a suicide promoter or any combination thereof.

In yet another embodiment, the vector expressing the CAR can be further modified to include one or more operative elements to control the expression of CAR T cells, or to eliminate CAR-T cells by virtue of a suicide switch. The suicide switch can include, for example, an apoptosis inducing signaling cascade or a drug that induces cell death. In a preferred embodiment, the vector expressing the CAR can be further modified to express an enzyme such thymidine kinase (TK) or cytosine deaminase (CD).

In another aspect, host cells including the nucleic acid molecule encoding the CAR are also provided. In some embodiments, the host cell is a T cell, such as a primary T cell obtained from a subject. In one embodiment, the host cell is a CD8⁺ T cell.

In yet another aspect, a pharmaceutical composition is provided comprising an anti-tumor effective amount of a population of human T cells, wherein the T cells comprise a nucleic acid sequence that encodes a chimeric antigen receptor (CAR) comprising the amino acid sequence of SEQ ID NO. 2 and 4, wherein the CAR comprises at least one extracellular antigen binding domain comprising a fully human CD20/CD19 antigen binding domain, at least one linker domain, at least one transmembrane domain, and at least one intracellular signaling domain, wherein the T cells are T cells of a human having a cancer. The cancer includes, inter alia, a hematological cancer such as leukemia (e.g., chronic lymphocytic leukemia (CLL), acute lymphocytic leukemia (ALL), or chronic myelogenous leukemia (CML), lymphoma (e.g., mantle cell lymphoma, non-Hodgkin's lymphoma or Hodgkin's lymphoma) or multiple myeloma, or a combination thereof.

In yet another aspect, a pharmaceutical composition is provided comprising an anti-tumor effective amount of a population of human T cells, wherein the T cells comprise a nucleic acid sequence that encodes a chimeric antigen receptor (CAR) comprising the amino acid sequence of SEQ ID NO. 49, wherein the CAR comprises at least one extracellular antigen binding domain comprising a fully human CD20/CD19 antigen binding domain, at least one linker domain, at least one transmembrane domain, and at least one intracellular signaling domain, wherein the T cells are T cells of a human having a cancer. The cancer includes, inter alia, a hematological cancer such as leukemia (e.g., chronic lymphocytic leukemia (CLL), acute lymphocytic leukemia (ALL), or chronic myelogenous leukemia (CML), lymphoma (e.g., mantle cell lymphoma, non-Hodgkin's lymphoma or Hodgkin's lymphoma) or multiple myeloma, or a combination thereof.

In one embodiment, a pharmaceutical composition is provided wherein the at least one transmembrane domain of the CAR contains a transmembrane domain of a protein selected from the group consisting of the alpha, beta or zeta chain of the T-cell receptor, CD28, CD3 epsilon, CD45, CD4, CD5, CD8, CD9, CD16, CD22, Mesothelin, CD33, CD37, CD64, CD80, CD83, CD86, CD134, CD137, CD154, TNFRSF19, or a combination thereof.

In another embodiment, a pharmaceutical composition is provided wherein the human cancer includes an adult carcinoma comprising oral and pharynx cancer (tongue, mouth, pharynx, head and neck), digestive system cancers (esophagus, stomach, small intestine, colon, rectum, anus, liver, intrahepatic bile duct, gallbladder, pancreas), respiratory system cancers (larynx, lung and bronchus), bones and joint cancers, soft tissue cancers, skin cancers (melanoma, basal and squamous cell carcinoma), pediatric tumors (neuroblastoma, rhabdomyosarcoma, osteosarcoma, Ewing's sarcoma), tumors of the central nervous system (brain, astrocytoma, glioblastoma, glioma), and cancers of the breast, the genital system (uterine cervix, uterine corpus, ovary, vulva, vagina, prostate, testis, penis, endometrium), the urinary system (urinary bladder, kidney and renal pelvis, ureter), the eye and orbit, the endocrine system (thyroid), and the brain and other nervous system, or any combination thereof.

In yet another embodiment, a pharmaceutical composition is provided comprising an anti-tumor effective amount of a population of human T cells of a human having a cancer wherein the cancer is a refractory cancer non-responsive to one or more chemotherapeutic agents. The cancer includes hematopoietic cancer, myelodysplastic syndrome pancreatic cancer, head and neck cancer, cutaneous tumors, minimal residual disease (MRD) in acute lymphoblastic leukemia (ALL), acute myeloid leukemia (AML), adult B cell malignancies including, CLL (Chronic lymphocytic leukemia), CML (chronic myelogenous leukemia), non-Hodgkin's lymphoma (NHL), pediatric B cell malignancies (including B lineage ALL (acute lymphocytic leukemia)), multiple myeloma lung cancer, breast cancer, ovarian cancer, prostate cancer, colon cancer, melanoma or other hematological cancer and solid tumors, or any combination thereof.

In another aspect, methods of making CAR-containing T cells (hereinafter “CAR-T cells”) are provided. The methods include transducing a T cell with a vector or nucleic acid molecule encoding a disclosed CAR that specifically binds CD19 and/or CD20, thereby making the CAR-T cell.

In yet another aspect, a method of generating a population of RNA-engineered cells is provided that comprises introducing an in vitro transcribed RNA or synthetic RNA of a nucleic acid molecule encoding a disclosed CAR into a cell of a subject, thereby generating a CAR cell.

In one embodiment, the disease, disorder or condition associated with the expression of CD19 is cancer including hematopoietic cancer, myelodysplastic syndrome pancreatic cancer, head and neck cancer, cutaneous tumors, minimal residual disease (MRD) in acute lymphoblastic leukemia (ALL), acute myeloid leukemia (AML), adult B cell malignancies including, CLL (Chronic lymphocytic leukemia), CML (chronic myelogenous leukemia), non-Hodgkin's lymphoma (NHL), pediatric B cell malignancies (including B lineage ALL (acute lymphocytic leukemia)), multiple myeloma lung cancer, breast cancer, ovarian cancer, prostate cancer, colon cancer, melanoma or other hematological cancer and solid tumors, or any combination thereof.

In another embodiment, a method of blocking T-cell inhibition mediated by a CD19− and/or CD20 expressing cell and altering the tumor microenvironment to inhibit tumor growth in a mammal, is provided comprising administering to the mammal an effective amount of a composition comprising a CAR comprising the amino acid sequence selected from the group consisting of SEQ ID NOs: 2, 4, and 49. In one embodiment, the cell is selected from the group consisting of a CD19 and/or CD20-expressing tumor cell, a tumor-associated macrophage, and any combination thereof.

In another embodiment, a method of inhibiting, suppressing or preventing immunosuppression of an anti-tumor or anti-cancer immune response in a mammal, is provided comprising administering to the mammal an effective amount of a composition comprising a CAR selected from the group consisting of SEQ ID NOs: 2, 4, and 49. In one embodiment, the CAR inhibits the interaction between a first cell with a T cell, wherein the first cell is selected from the group consisting of a CD19 and/or CD20-expressing tumor cell, a tumor-associated macrophage, and any combination thereof.

In another aspect, a method is provided for inducing an anti-tumor immunity in a mammal comprising administering to the mammal a therapeutically effective amount of a T cell transduced with vector or nucleic acid molecule encoding a disclosed CAR.

In another embodiment, a method of treating or preventing cancer in a mammal is provided comprising administering to the mammal one or more of the disclosed CARs, in an amount effective to treat or prevent cancer in the mammal. The method includes administering to the subject a therapeutically effective amount of host cells expressing a disclosed CAR that specifically binds CD19 and/or CD20 and/or one or more of the aforementioned antigens, under conditions sufficient to form an immune complex of the antigen binding domain on the CAR and the extracellular domain of CD19 and/or CD20 and/or one or more of the aforementioned antigens in the subject.

In yet another embodiment, a method is provided for treating a mammal having a disease, disorder or condition associated with an elevated expression of a tumor antigen, the method comprising administering to the subject a pharmaceutical composition comprising an anti-tumor effective amount of a population of T cells, wherein the T cells comprise a nucleic acid sequence that encodes a chimeric antigen receptor (CAR), wherein the CAR includes at least one extracellular CD19 and/or CD20 antigen binding domain, or any combination thereof, at least one linker or spacer domain, at least one transmembrane domain, at least one intracellular signaling domain, and wherein the T cells are T cells of the subject having cancer.

In yet another embodiment, a method is provided for treating cancer in a subject in need thereof comprising administering to the subject a pharmaceutical composition comprising an anti-tumor effective amount of a population of T cells, wherein the T cells comprise a nucleic acid sequence that encodes a chimeric antigen receptor (CAR), wherein the CAR comprises the amino acid sequence of SEQ ID NOs. 2 and 4, or any combination thereof, at least one linker or spacer domain, at least one transmembrane domain, at least one intracellular signaling domain, wherein the T cells are T cells of the subject having cancer. In some embodiments of the aforementioned methods, the at least one transmembrane domain comprises a transmembrane the alpha, beta or zeta chain of the T-cell receptor, CD28, CD3 epsilon, CD45, CD4, CD5, CD8, CD9, CD16, CD19, CD22, Mesothelin, CD33, CD37, CD64, CD80, CD83, CD86, CD134, CD137, CD154, TNFRSF16, TNFRSF19, or a combination thereof.

In yet another embodiment, a method is provided for treating cancer in a subject in need thereof comprising administering to the subject a pharmaceutical composition comprising an anti-tumor effective amount of a population of T cells, wherein the T cells comprise a nucleic acid sequence that encodes a chimeric antigen receptor (CAR), wherein the CAR comprises the amino acid sequence of SEQ ID NO. 49, at least one linker or spacer domain, at least one transmembrane domain, at least one intracellular signaling domain, wherein the T cells are T cells of the subject having cancer. In some embodiments of the aforementioned methods, the at least one transmembrane domain comprises a transmembrane the alpha, beta or zeta chain of the T-cell receptor, CD28, CD3 epsilon, CD45, CD4, CD5, CD8, CD9, CD16, CD19, CD22, Mesothelin, CD33, CD37, CD64, CD80, CD83, CD86, CD134, CD137, CD154, TNFRSF16, TNFRSF19, or a combination thereof.

In yet another embodiment, a method is provided for generating a persisting population of genetically engineered T cells in a human diagnosed with cancer. In one embodiment, the method comprises administering to a human a T cell genetically engineered to express a CAR wherein the CAR comprises the amino acid sequence of SEQ ID NOs. 2 and 4, or any combination thereof, at least one transmembrane domain, and at least one intracellular signaling domain wherein the persisting population of genetically engineered T cells, or the population of progeny of the T cells, persists in the human for at least one month, two months, three months, four months, five months, six months, seven months, eight months, nine months, ten months, eleven months, twelve months, two years, or three years after administration.

In yet another embodiment, a method is provided for generating a persisting population of genetically engineered T cells in a human diagnosed with cancer. In one embodiment, the method comprises administering to a human a T cell genetically engineered to express a CAR wherein the CAR comprises the amino acid sequence of SEQ ID NO. 49, at least one transmembrane domain, and at least one intracellular signaling domain wherein the persisting population of genetically engineered T cells, or the population of progeny of the T cells, persists in the human for at least one month, two months, three months, four months, five months, six months, seven months, eight months, nine months, ten months, eleven months, twelve months, two years, or three years after administration.

In one embodiment, the progeny T cells in the human comprise a memory T cell. In another embodiment, the T cell is an autologous T cell.

In all of the aspects and embodiments of methods described herein, any of the aforementioned cancers, diseases, disorders or conditions associated with an elevated expression of a tumor antigen that may be treated or prevented or ameliorated using one or more of the CARs disclosed herein.

In yet another aspect, a kit is provided for making a chimeric antigen receptor T-cell as described supra or for preventing, treating, or ameliorating any of the cancers, diseases, disorders or conditions associated with an elevated expression of a tumor antigen in a subject as described supra, comprising a container comprising any one of the nucleic acid molecules, vectors, host cells, or compositions disclosed supra or any combination thereof, and instructions for using the kit.

It will be understood that the CARs, host cells, nucleic acids, and methods are useful beyond the specific aspects and embodiments that are described in detail herein. The foregoing features and advantages of the disclosure will become more apparent from the following detailed description, which proceeds with reference to the accompanying figures.

### BRIEF DESCRIPTION OF THE FIGURES

FIGS. 1A-1B depict CD20 and CD19 dual targeting CAR structures and surface expression in human primary T cells. FIG. 1A) CD20 and CD19 dual targeting CARs were designed as tandem or bicistronic constructs. Tandem CAR constructs comprised of a fully human 20-19 tandem scFv targeting domain, a hinge and transmembrane domain, a single 4-1BB or tandem CD28_4-1BB co-stimulatory domain and a CD3ζ activation domain. Construct pLTG1497 with mouse tandem scFv were served as control. Duo CAR constructs contained a mono CD20 CAR, followed by 2A sequence, and a monoCD19 CAR with different co-stimulatory domain or transmembrane domain. FIG. 1B) Primary T cells from healthy donor were activated with TransAct in the presence of IL-2, and transduced with lentiviral vectors encoding CAR20_19 constructs. Transduced T cells were assayed for CAR surface expression with CD19 Fc staining followed by anti-Fc-AF647 with flow cytometry. UTD—untransduced control.

FIGS. 2A-2D depict the cytotoxicity of CAR20_19 constructs in vitro. Luciferase-based cytotoxicity assays were performed using CD19+ CD20+ tumor lines: FIG. 2A) Raji, FIG. 2B) Nalm-6, FIG. 2C) Reh and CD19-CD20− tumor line FIG. 2D) 293T. All target lines were stably transduced with firefly luciferase. CAR T cells and tumor cells were co-cultured overnight at the indicated effector to target (E:T) ratios: 2.5:1, 5:1, or 10:1. Percentage specific target lysis was assessed by luminometry. Data represented one independent experiment from 2 different donors. Mean±SD of three technical replicates. One representative experiment of 4 separate donors was shown in the panel.

FIGS. 3A-3C depict CAR T cytokine release in response to Raji lymphoma cell lines. Culture supernatants of CAR T cells was evaluated after overnight incubation alone or with CD19+ CD20+ Raji target cells at E:T ratio of 10. Cytokine production of FIG. 3A) IL-2, FIG. 3B) IFNγ, and FIG. 3C) TNFα, were analyzed by ELISA. Mean±SD of three technical replicates. Data represented one independent experiment from two separate donors.

FIGS. 4A-4C depict the cytotoxicity of CAR20_19 constructs with long term target cell stimulation. FIG. 4A) Schema of long term stimulation with CD19+ CD20+ Raji lymphoma cells. At round1, CAR T cells were cocultured with Raji cells at E:T 0.3:1 for 7 days. The remaining CAT T cells and Raji cells were measured with flow cytometry, and re-adjusted to E:T 0.15:1 for the second round. FIG. 4B) Remaining Raji percentage were analyzed using GFP by flow at day 4, day7 and day10. FIG. 4C) T cell number were measured by flow cytometry with CountBright™ Absolute Counting Beads. Fold expansion compared to input T cell were calculated and plotted. Arrow indicated coculture starting point for each round. Data were representative results from one of the two donors.

FIGS. 5A-5C depict the experimental design and timeline of the in vivo challenge and rechallenge study of CAR T cells in mouse NSG Raji lymphoma xenograft (FIG. 5A), representative bioluminescence images of tumor progression in mice on the study (FIG. 5B), and summary of bioluminescence measurements representing the tumor progression kinetics over the course of the initial challenge and the rechallenge study (FIG. 5C).

### DETAILED DESCRIPTION

### Definitions

As used herein, the singular forms “a,” “an,” and “the,” refer to both the singular as well as plural, unless the context clearly indicates otherwise. For example, the term “an antigen” includes single or plural antigens and can be considered equivalent to the phrase “at least one antigen.” As used herein, the term “comprises” means “includes.” Thus, “comprising an antigen” means “including an antigen” without excluding other elements. The phrase “and/or” means “and” or “or.” It is further to be understood that any and all base sizes or amino acid sizes, and all molecular weight or molecular mass values, given for nucleic acids or polypeptides are approximate, and are provided for descriptive purposes, unless otherwise indicated. Although many methods and materials similar or equivalent to those described herein can be used, particular suitable methods and materials are described below. In case of conflict, the present specification, including explanations of terms, will control. In addition, the materials, methods, and examples are illustrative only and not intended to be limiting. To facilitate review of the various embodiments, the following explanations of terms are provided:

The term “about” when referring to a measurable value such as an amount, a temporal duration, and the like, is meant to encompass variations of .+−0.20% or in some instances .+−0.10%, or in some instances .+−0.5%, or in some instances .+−0.1%, or in some instances .+−0.0.1% from the specified value, as such variations are appropriate to perform the disclosed methods.

Unless otherwise noted, the technical terms herein are used according to conventional usage. Definitions of common terms in molecular biology can be found in Benjamin Lewin, Genes V7, published by Oxford University Press, 1999; Kendrew et al. (eds.), The Encyclopedia of Molecular Biology, published by Blackwell Science Ltd., 1994; and Robert A. Meyers (ed.), Molecular Biology and Biotechnology: a Comprehensive Desk Reference, published by VCH Publishers, Inc., 1995; and other similar references.

The present disclosure provides for fully human CD20/CD19 antibodies or fragments thereof as well as chimeric antigen receptors (CARs) having such fully human CD20/CD19 antigen binding domains. The enhancement of the functional activity of the CAR directly relates to the enhancement of functional activity of the CAR-expressing T cell. As a result of one or more of these modifications, the CARs exhibit both a high degree of cytokine-induced cytolysis and cell surface expression on transduced T cells, along with an increased level of in vivo T cell expansion and persistence of the transduced CAR-expressing T cell. The CARs of the present disclosure are advantageous in that one CART lentiviral product may be utilized to treat multiple patient populations (i.e. CD19+, CD20+ or double CD19+ CD20+ cancer patients), which allows flexibility in circumstances where resources are limited.

The unique ability to combine functional moieties derived from different protein domains has been a key innovative feature of Chimeric Antigen Receptors (CARs). The choice of each of these protein domains is a key design feature, as is the way in which they are specifically combined. Each design domain is an essential component that can be used across different CAR platforms to engineer the function of lymphocytes. For example, the choice of the extracellular binding domain can make an otherwise ineffective CAR be effective.

The invariable framework components of the immunoglobulin-derived protein sequences used to create the extracellular antigen binding domain of a CAR can either be entirely neutral, or they can self-associate and drive the T cell to a state of metabolic exhaustion, thus making the therapeutic T cell expressing that CAR far less effective. This occurs independently of the antigen binding function of this CAR domain. Furthermore, the choice of the intracellular signaling domain(s) also can govern the activity and the durability of the therapeutic lymphocyte population used for immunotherapy. While the ability to bind target antigen and the ability to transmit an activation signal to the T cell through these extracellular and intracellular domains, respectively, are important CAR design aspects, what has also become apparent is that the choice of the source of the extracellular antigen binding fragments can have a significant effect on the efficacy of the CAR and thereby have a defining role for the function and clinical utility of the CAR.

The CARs disclosed herein are expressed at a high level in a cell. A cell expressing the CAR has a high in vivo proliferation rate, produces large amounts of cytokines, and has a high cytotoxic activity against a cell having, on its surface, a fully human CD20/CD19 antigen to which a CAR binds. The use of an extracellular fully human CD20/CD19 antigen binding domain results in generation of a CAR that functions better in vivo, while avoiding the induction of anti-CAR immunity in the host immune response and the killing of the CAR T cell population. The CARs expressing the extracellular fully human CD20/CD19 ScFv antigen binding domain exhibit superior activities/properties including i) prevention of poor CAR T persistence and function as seen with mouse-derived binding sequences; ii) lack of regional (i.e. intrapleural) delivery of the CAR to be efficacious; and iii) ability to generate CAR T cell designs based both on binders with high and low affinity to fully human CD20/CD19. This latter property allows investigators to better tune efficacy vs toxicity, and/or tissue specificity of the CAR T product, since lower-affinity binders may have higher specificity to tumors vs normal tissues due to higher expression of fully human CD20/CD19 on tumors than normal tissue, which may prevent on-target off tumor toxicity and bystander cell killing.

What follows is a detailed description of the inventive CARs including a description of their extracellular fully human CD20/CD19 antigen binding domain, the transmembrane domain and the intracellular domain, along with additional description of the CARs, antibodies and antigen binding fragments thereof, conjugates, nucleotides, expression, vectors, and host cells, methods of treatment, compositions, and kits employing the disclosed CARs.

In each of the aforementioned aspects and embodiments described supra, for example, scFv binders have been created for mesothelin as disclosed in Applicant's issued U.S. Pat. No. 10,183,993, entitled Compositions and Methods for Treating Cancer with Anti-Mesothelin Immunotherapy, and assigned Lentigen Technology, Inc. matter number LEN_017, nucleotide sequence ScFv antigen SEQ ID NO: 149 and amino acid sequence SEQ ID NO: 150, respectively, that can be incorporated into functional CARs, nucleotide sequence SEQ ID NO: 39 and amino acid sequence SEQ ID NO: 40, respectively, and that can thereby be incorporated into a DuoCAR therapy.

In each of the aforementioned aspects and embodiments described supra, in addition to scFv sequences, single chain antigen binders (as opposed to scFv) can be incorporated into a single, tandem, DuoCAR, or multi-targeting CAR application. For example, the CD33-specific heavy chain only binder as disclosed in Applicant's issued U.S. Pat. No. 10,426,797, entitled Compositions and Methods For Treating Cancer With Anti-CD33 Immunotherapy, and assigned Lentigen Technology, Inc. matter number LEN_018, nucleotide sequence SEQ ID NO: 41 and amino acid sequence SEQ ID NO: 42, respectively, can be incorporated into a functional CAR, LTG1906, nucleotide sequence SEQ ID NO: 43 and amino acid sequence SEQ ID NO: 44, respectively, that targets CD33-expressing malignancies.

In each of the aforementioned aspects and embodiments described supra, one example of a single, tandem, DuoCAR, or multi-targeting CAR therapeutic application would be the treatment of leukemia that expresses the CD19, CD20, and TSLPR antigens. In this case, LTG1496 or LTG1497 (SEQ ID NOs: 35, 26, respectively) could be combined with a TSLPR-specific CAR (LTG1789), SEQ ID NO: 47 and amino acid sequence SEQ ID NO: 48, respectively, that had been created from TSLPR-specific scFV domains, nucleotide sequence SEQ ID NO: 45 and amino acid sequence SEQ ID NO: 46.

In each of the aforementioned aspects and embodiments described supra, another example of a single, tandem, DuoCAR, or multi-targeting CAR therapeutic application would be the treatment of cancer that expresses the CD38 antigen. For instance, the CD38-specific binders as disclosed in Applicant's issued U.S. Pat. No. 11,103,533; entitled Compositions and Methods For Treating Cancer With Anti-CD38 Immunotherapy; as filed on Nov. 30, 2018; and assigned Lentigen Technology, Inc. matter number LEN_026; can be incorporated into one or more functional CARs that target CD38-expressing malignancies, as disclosed in Applicant's issued U.S. Pat. No. 11,103,533, the entirety of which is incorporated by reference herein.

In each of the aforementioned aspects and embodiments described supra, another example of a single, tandem, DuoCAR, or multi-targeting CAR therapeutic application would be the treatment of cancer that expresses the CD123 antigen. For instance, the CD123-specific binders as disclosed in Applicant's issued U.S. Pat. No. 10,844,128; entitled Compositions and Methods For Treating Cancer With Anti-CD123 Immunotherapy; as filed on Sep. 20, 2019; and assigned Lentigen Technology, Inc. matter number LEN_024; and claiming priority to Provisional Patent Application No. 62/734,106; as filed on Sep. 20, 2018; can be incorporated into one or more functional CARs that target CD123-expressing malignancies as disclosed in Applicant's issued U.S. Pat. No. 10,844,128, the entirety of which is incorporated by reference herein.

In each of the aforementioned aspects and embodiments described supra, another example of a single, tandem, DuoCAR, or multi-targeting CAR therapeutic application would be the treatment of cancer that expresses the CD123 antigen. For instance, the CD123-specific binders as disclosed in Applicant's U.S. co-pending patent application Ser. No. 17/685,132; entitled Compositions and Methods For Treating Cancer With Anti-CD123 Immunotherapy; as filed on Mar. 2, 2022; and assigned Lentigen Technology, Inc. matter number MBG_99; can be incorporated into one or more functional CARs that target CD123-expressing malignancies, as disclosed in Applicant's co-pending U.S. patent application Ser. No. 17/685,132, the entirety of which is incorporated by reference herein.

In each of the aforementioned aspects and embodiments described supra, another example of a single, tandem, DuoCAR, or multi-targeting CAR therapeutic application would be the treatment of cancer that expresses the BCMA antigen. For instance, the BCMA-specific binders as disclosed in Applicant's issued U.S. Pat. No. 11,052,112; entitled Fully Human BCMA CAR T Cells for the Treatment of Multiple Myeloma and Other BCMA-Positive Malignancies, as filed on May 30, 2019; and assigned Lentigen Technology, Inc. matter number MBG_13; can be incorporated into one or more functional CARs that target BCMA-expressing malignancies, as disclosed in Applicant's issued U.S. Pat. No. 11,052,112, the entirety of which is incorporated by reference herein.

In each of the aforementioned aspects and embodiments described supra, another example of a single, tandem, DuoCAR, or multi-targeting CAR therapeutic application would be the treatment of solid tumor cancers. For instance, the specific binders as disclosed in Applicant's co-pending U.S. Provisional Patent Application No. 63/393,088; entitled Chimeric Antigen Receptor Therapies for Treating Solid Tumors; as filed on Jul. 28, 2022; and assigned Lentigen Technology, Inc. matter number MBG_106; can be incorporated into one or more functional CARs that target solid tumors, as disclosed in Applicant's co-pending U.S. Provisional Patent Application No. 63/393,088, the entirety of which is incorporated by reference herein.

In each of the aforementioned aspects and embodiments described supra, examples of tandem-CARs (containing 2 scFv domains, as described in nucleotide sequence SEQ ID NO: 23 and amino acid sequence SEQ ID NO: 24) on which this technology is based include the CD20_CD19 CAR LTG1497, nucleotide sequence SEQ ID NO: 25 and amino acid sequence SEQ ID NO: 26. In some cases reversing the order of the two binders may provide a better DuoCAR expression in target cells. Thus, LTG1497, where the CD19 scFv is more proximal, as shown in nucleotide sequence SEQ ID NO: 25 and amino acid sequence SEQ ID NO: 26; and LTG1496 where the CD19 scFV is more distal to the membrane, as shown in nucleotide sequence SEQ ID NO: 33 and amino acid sequence SEQ ID NO: 34, can both be used as one of the members of a DuoSet comprising a DuoCAR.

A. Chimeric Antigen Receptors (CARs)

The CARs disclosed herein comprise at least one fully human CD20/CD19 antigen binding domain capable of binding to fully human CD20/CD19, at least one transmembrane domain, and at least one intracellular domain.

A chimeric antigen receptor (CAR) is an artificially constructed hybrid protein or polypeptide containing the antigen binding domains of an antibody (e.g., single chain variable fragment (ScFv)) linked to T-cell signaling domains via the transmembrane domain. Characteristics of CARs include their ability to redirect T-cell specificity and reactivity toward a selected target in a non-MHC-restricted manner, and exploiting the antigen-binding properties of monoclonal antibodies. The non-MHC-restricted antigen recognition gives T cells expressing CARs the ability to recognize antigen independent of antigen processing, thus bypassing a major mechanism of tumor escape. Moreover, when expressed in T-cells, CARs advantageously do not dimerize with endogenous T cell receptor (TCR) alpha and beta chains.

As disclosed herein, the intracellular T cell signaling domains of the CARs can include, for example, a T cell receptor signaling domain, a T cell costimulatory signaling domain, or both. The T cell receptor signaling domain refers to a portion of the CAR comprising the intracellular domain of a T cell receptor, such as, for example, and not by way of limitation, the intracellular portion of the CD3 zeta protein. The costimulatory signaling domain refers to a portion of the CAR comprising the intracellular domain of a costimulatory molecule, which is a cell surface molecule other than an antigen receptor or their ligands that are required for an efficient response of lymphocytes to antigen.

1. Extracellular Domain

In one embodiment, the CAR comprises a target-specific binding element otherwise referred to as an antigen binding domain or moiety. The choice of domain depends upon the type and number of ligands that define the surface of a target cell. For example, the antigen binding domain may be chosen to recognize a ligand that acts as a cell surface marker on target cells associated with a particular disease state. Thus examples of cell surface markers that may act as ligands for the antigen binding domain in the CAR include those associated with viral, bacterial and parasitic infections, autoimmune disease and cancer cells.

In one embodiment, the CAR can be engineered to target a tumor antigen of interest by way of engineering a desired antigen binding domain that specifically binds to an antigen on a tumor cell. Tumor antigens are proteins that are produced by tumor cells that elicit an immune response, particularly T-cell mediated immune responses. The selection of the antigen binding domain will depend on the particular type of cancer to be treated. Tumor antigens include, for example, a glioma-associated antigen, carcinoembryonic antigen (CEA), .beta.-human chorionic gonadotropin, alphafetoprotein (AFP), lectin-reactive AFP, thyroglobulin, RAGE-1, MN-CA IX, human telomerase reverse transcriptase, RU1, RU2 (AS), intestinal carboxyl esterase, mut hsp70-2, M-CSF, prostase, prostate-specific antigen (PSA), PAP, NY-ESO-1, LAGE-1a, p53, prostein, PSMA, Her2/neu, survivin and telomerase, prostate-carcinoma tumor antigen-1 (PCTA-1), MAGE, ELF2M, neutrophil elastase, ephrinB2, CD22, insulin growth factor (IGF)-I, IGF-II, IGF-I receptor and fully human CD20/CD19. The tumor antigens disclosed herein are merely included by way of example. The list is not intended to be exclusive and further examples will be readily apparent to those of skill in the art.

In one embodiment, the tumor antigen comprises one or more antigenic cancer epitopes associated with a malignant tumor. Malignant tumors express a number of proteins that can serve as target antigens for an immune attack. These molecules include, but are not limited to, tissue-specific antigens such as MART-1, tyrosinase and GP 100 in melanoma and prostatic acid phosphatase (PAP) and prostate-specific antigen (PSA) in prostate cancer. Other target molecules belong to the group of transformation-related molecules such as the oncogene HER-2/Neu/ErbB-2. Yet another group of target antigens are onco-fetal antigens such as carcinoembryonic antigen (CEA). In B-cell lymphoma the tumor-specific idiotype immunoglobulin constitutes a truly tumor-specific immunoglobulin antigen that is unique to the individual tumor. B-cell differentiation antigens such as CD19, CD20, CD22, BCMA, ROR1, and CD37 are other candidates for target antigens in B-cell lymphoma. Some of these antigens (CEA, HER-2, CD19, CD20, idiotype) have been used as targets for passive immunotherapy with monoclonal antibodies with limited success.

In one preferred embodiment, the tumor antigen is fully human CD20/CD19 and the tumors associated with expression of fully human CD20/CD19 comprise lung mesothelioma, ovarian, and pancreatic cancers that express high levels of the extracellular protein fully human CD20/CD19, or any combination thereof.

The type of tumor antigen may also be a tumor-specific antigen (TSA) or a tumor-associated antigen (TAA). A TSA is unique to tumor cells and does not occur on other cells in the body. A TAA is not unique to a tumor cell and instead is also expressed on a normal cell under conditions that fail to induce a state of immunologic tolerance to the antigen. The expression of the antigen on the tumor may occur under conditions that enable the immune system to respond to the antigen. TAAs may be antigens that are expressed on normal cells during fetal development when the immune system is immature and unable to respond or they may be antigens that are normally present at extremely low levels on normal cells but which are expressed at much higher levels on tumor cells.

Non-limiting examples of TSAs or TAAs include the following: Differentiation antigens such as MART-1/MelanA (MART-I), gp100 (Pmel 17), tyrosinase, TRP-1, TRP-2 and tumor-specific multi-lineage antigens such as MAGE-1, MAGE-3, BAGE, GAGE-1, GAGE-2, p15; overexpressed embryonic antigens such as CEA; overexpressed oncogenes and mutated tumor-suppressor genes such as p53, Ras, HER-2/neu: unique tumor antigens resulting from chromosomal translocations; such as BCR-ABL, E2A-PRL, H4-RET, IGH-IGK, MYL-RAR; and viral antigens, such as the Epstein Barr virus antigens EBVA and the human papillomavirus (HPV) antigens E6 and E7. Other large, protein-based antigens include TSP-180, MAGE-4, MAGE-5, MAGE-6, RAGE, NY-ESO, p185erbB2, p180erbB-3, c-met, nm-23H1, PSA, TAG-72, CA 19-9, CA 72-4, CAM 17.1, NuMa, K-ras, beta-Catenin, CDK4, Mum-1, p 15, p 16, 43-9F, 5T4, 791Tgp72, alpha-fetoprotein, beta-HCG, BCA225, BTAA, CA 125, CA 15-3\CA 27.29\BCAA, CA 195, CA 242, CA-50, CAM43, CD68\P1, CO-029, FGF-5, G250, Ga733\EpCAM, HTgp-175, M344, MA-50, MG7-Ag, MOV18, NB/70K, NY-CO-1, RCAS1, SDCCAG16, TA-90\Mac-2 binding protein\cyclophilin C-associated protein, TAAL6, TAG72, TLP, and TPS.

In one embodiment, the antigen binding domain portion of the CAR targets an antigen that includes but is not limited to CD19, CD20, CD22, ROR1, CD33, CD38, CD123, CD138, BCMA, c-Met, PSMA, Glycolipid F77, EGFRvIII, GD-2, FGFR4, TSLPR, NY-ESO-1 TCR, MAGE A3 TCR, and the like.

In a preferred embodiment, the antigen binding domain portion of the CAR targets the extracellular CD20/CD19 antigen.

In a preferred embodiment, the antigen binding domain portion of the CAR targets the extracellular CD20 antigen.

In one preferred embodiment, the isolated nucleic acid molecule encoding the extracellular CD20 scFv antigen binding domain comprises a nucleotide sequence of SEQ ID NO: 1, or a sequence with 85%, 90%, 95%, 96%, 97%, 98% or 99% identity thereof. In one embodiment, an isolated nucleic acid molecule is provided wherein the encoded extracellular CD20 scFv antigen binding domain comprises an amino acid sequence of SEQ ID NO: 2, or an amino acid sequence with 85%, 90%, 95%, 96%, 97%, 98% or 99% identity to an amino acid sequence of SEQ ID NO: 2.

In a preferred embodiment, the antigen binding domain portion of the CAR targets the extracellular CD19 antigen.

In one preferred embodiment, the isolated nucleic acid molecule encoding the extracellular CD19 scFv antigen binding domain comprises a nucleotide sequence of SEQ ID NO: 3, or a sequence with 85%, 90%, 95%, 96%, 97%, 98% or 99% identity thereof. In one embodiment, an isolated nucleic acid molecule is provided wherein the encoded extracellular CD19 scFv antigen binding domain comprises an amino acid sequence of SEQ ID NO: 4, or an amino acid sequence with 85%, 90%, 95%, 96%, 97%, 98% or 99% identity to an amino acid sequence of SEQ ID NO: 4.

In a preferred embodiment, the antigen binding domain portion of the CAR targets the extracellular CD20 antigen and CD19 antigen.

In one preferred embodiment, the isolated nucleic acid molecule encoding the extracellular CD20/CD19 scFv antigen binding domain comprises a nucleotide sequence of SEQ ID NO: 48, or a sequence with 85%, 90%, 95%, 96%, 97%, 98% or 99% identity thereof. In one embodiment, an isolated nucleic acid molecule is provided wherein the encoded extracellular CD20/CD19 scFv antigen binding domain comprises an amino acid sequence of SEQ ID NO: 49, or an amino acid sequence with 85%, 90%, 95%, 96%, 97%, 98% or 99% identity to an amino acid sequence of SEQ ID NO: 49.

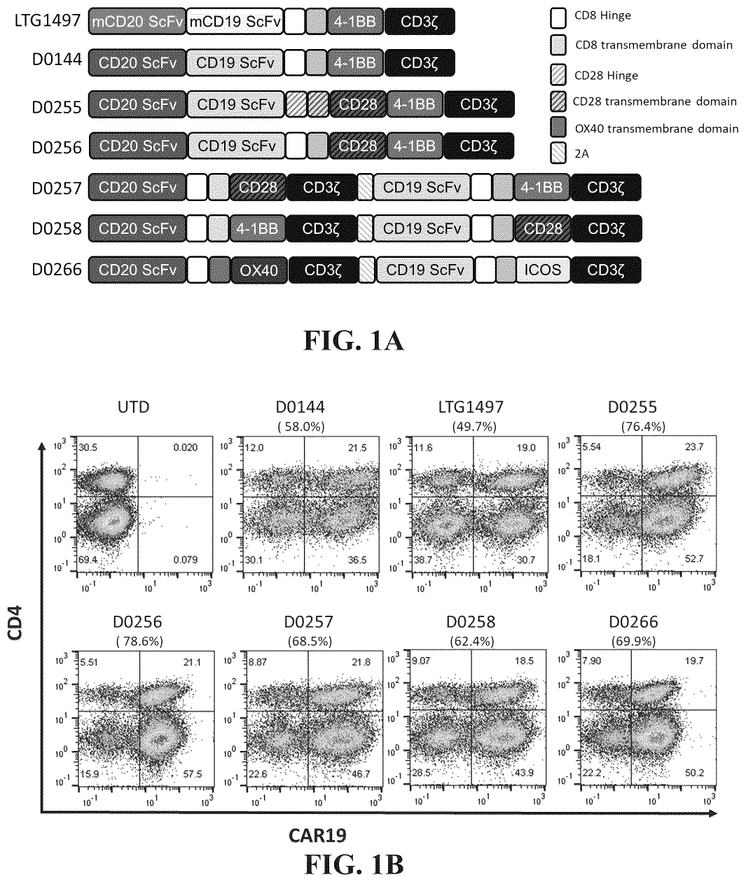

In the various embodiments of the fully human CD20/CD19-specific multi-targeting, tandem, and bicistronic CARs disclosed herein, the general scheme is set forth in FIG. 1A and includes, from the N-terminus to the C-terminus, CAR targeting domain, transmembrane domain, co-stimulatory domain and CD3 zeta activating domain. In tandem CARs, a fully human, tandem CD20 and CD19 targeting domain is included. For bicistronic CARs, the first, CD20 targeting CAR is comprised of a fully human CD20-targeting domain, and is followed in frame by a second, CD19-targeting CAR comprised of a CD19-targeting domain, and a ribosomal skip element is inserted between the CD20 CAR and the CD19 CAR sequences to facilitate co-expression.

In one embodiment, the nucleic acid sequence encoding a CAR comprises the nucleic acid sequence of SEQ ID NO: 5 (D0144) (CD20_CD19 CD8 BBz), and encodes the CAR comprising the amino acid sequence as set forth in SEQ ID NO: 6.

In one embodiment, the nucleic acid sequence encoding a CAR comprises the nucleic acid sequence of SEQ ID NO: 5, or a sequence with 85%, 90%, 95%, 96%, 97%, 98% or 99% identity thereof, and encodes the CAR comprising the amino acid sequence as set forth in SEQ ID NO: 6 or a sequence with 85%, 90%, 95%, 96%, 97%, 98% or 99% identity thereof (D0144) (CD20_CD19 CD8 BBz).

In another embodiment, the nucleic acid sequence encoding a CAR comprises the nucleic acid sequence of SEQ ID NO: 7 (CAR D0255) (CD20_CD19 CD28 CD28 BBz), and encodes the CAR comprising the amino acid sequence as set forth in SEQ ID NO: 8 (CAR D0255) (CD20_CD19 CD28 CD28 BBz ).

In another embodiment, the nucleic acid sequence encoding a CAR comprises the nucleic acid sequence of SEQ ID NO: 7 or a sequence with 85%, 90%, 95%, 96%, 97%, 98% or 99% identity thereof, and encodes the CAR comprising the amino acid sequence as set forth in SEQ ID NO: 8 or a sequence with 85%, 90%, 95%, 96%, 97%, 98% or 99% identity thereof (CAR D0255) (CD20_CD19 CD28 CD28 BBz).

In one embodiment, the nucleic acid sequence encoding a CAR comprises the nucleic acid sequence of SEQ ID NO: 9 (CAR D0256) (CD20_CD19 CD8 CD28 BBz), and encodes the CAR comprising the amino acid sequence as set forth in SEQ ID NO: 10.

In one embodiment, the nucleic acid sequence encoding a CAR comprises the nucleic acid sequence of SEQ ID NO: 9, or a sequence with 85%, 90%, 95%, 96%, 97%, 98% or 99% identity thereof, and encodes the CAR comprising the amino acid sequence as set forth in SEQ ID NO: 10 or a sequence with 85%, 90%, 95%, 96%, 97%, 98% or 99% identity thereof (CAR D0256) (CD20_CD19 CD8 CD28 BBz).

In one embodiment, the nucleic acid sequence encoding a CAR comprises the nucleic acid sequence of SEQ ID NO: 13 (CAR D0257) (CD20 CD8 CD28z_CD19 CD8 BBz), and encodes the CAR comprising the amino acid sequence as set forth in SEQ ID NO: 14.

In one embodiment, the nucleic acid sequence encoding a CAR comprises the nucleic acid sequence of SEQ ID NO: 13, or a sequence with 85%, 90%, 95%, 96%, 97%, 98% or 99% identity thereof, and encodes the CAR comprising the amino acid sequence as set forth in SEQ ID NO: 14 or a sequence with 85%, 90%, 95%, 96%, 97%, 98% or 99% identity thereof (CAR D0257) (CD20 CD8 CD28z_CD19 CD8 BBz).

In one embodiment, the nucleic acid sequence encoding a CAR comprises the nucleic acid sequence of SEQ ID NO: 15 (CAR D0258) (CD20 CD8 BBz_CD19 CD8 CD28z), and encodes the CAR comprising the amino acid sequence as set forth in SEQ ID NO: 16.

In one embodiment, the nucleic acid sequence encoding a CAR comprises the nucleic acid sequence of SEQ ID NO: 15, or a sequence with 85%, 90%, 95%, 96%, 97%, 98% or 99% identity thereof, and encodes the CAR comprising the amino acid sequence as set forth in SEQ ID NO: 16 or a sequence with 85%, 90%, 95%, 96%, 97%, 98% or 99% identity thereof (CAR D0258) (CD20 CD8 BBz_CD19 CD8 CD28z).

In one embodiment, the nucleic acid sequence encoding a CAR comprises the nucleic acid sequence of SEQ ID NO: 17 (CAR D0266) (CD20 CD8 OX40 OX40z_CD19 CD8 ICOSz), and encodes the CAR comprising the amino acid sequence as set forth in SEQ ID NO: 18.

In one embodiment, the nucleic acid sequence encoding a CAR comprises the nucleic acid sequence of SEQ ID NO: 17, or a sequence with 85%, 90%, 95%, 96%, 97%, 98% or 99% identity thereof, and encodes the CAR comprising the amino acid sequence as set forth in SEQ ID NO: 18 or a sequence with 85%, 90%, 95%, 96%, 97%, 98% or 99% identity thereof (CAR D0266) (CD20 CD8 OX40 OX40z_CD19 CD8 ICOSz).

In one embodiment, the nucleic acid sequence encoding a CAR comprises the nucleic acid sequence of SEQ ID NO: 19 (CAR LTG1497) (mCD20_CD19 CD8 BBz), and encodes the CAR comprising the amino acid sequence as set forth in SEQ ID NO: 20.

In one embodiment, the nucleic acid sequence encoding a CAR comprises the nucleic acid sequence of SEQ ID NO: 19, or a sequence with 85%, 90%, 95%, 96%, 97%, 98% or 99% identity thereof, and encodes the CAR comprising the amino acid sequence as set forth in SEQ ID NO: 20 or a sequence with 85%, 90%, 95%, 96%, 97%, 98% or 99% identity thereof (CAR LTG1497) (mCD20_CD19 CD8 BBz).

The surface expression of anti-CD20/CD19 CARs incorporating single chain fragment variable (ScFv) sequences reactive with fully human CD20 and CD19 antigens, is shown in Example 1 infra, FIG. 1B. The expression level for each ScFv-containing CAR was determined by flow cytometric analysis of LV-transduced T cells from healthy donors using CD19 Fc recombinant protein, followed by anti Fc AF647 (APC). The ScFv-based anti-CD20/CD19 CAR constructs D0144, D0255, D0256, D0257, D0258, D0266, and comparator construct LTG1497, comprised of a murine CD20/CD19 scFv tandem targeting domain, were highly expressed in human primary T cells, both in CD4+ T and CD8+ T lineages, as compared to non-transduced T cell controls (range 49.7%-78.6% CAR+ of total T cells).

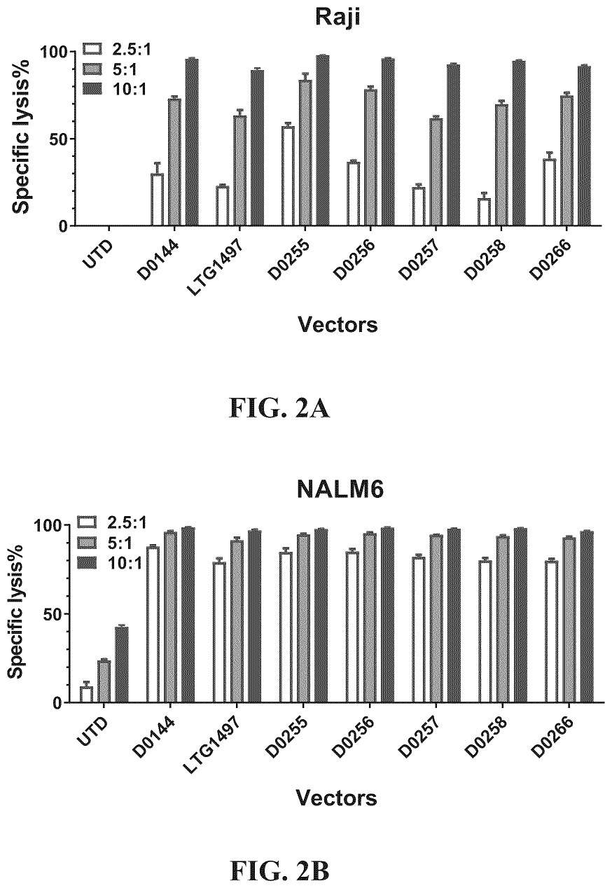

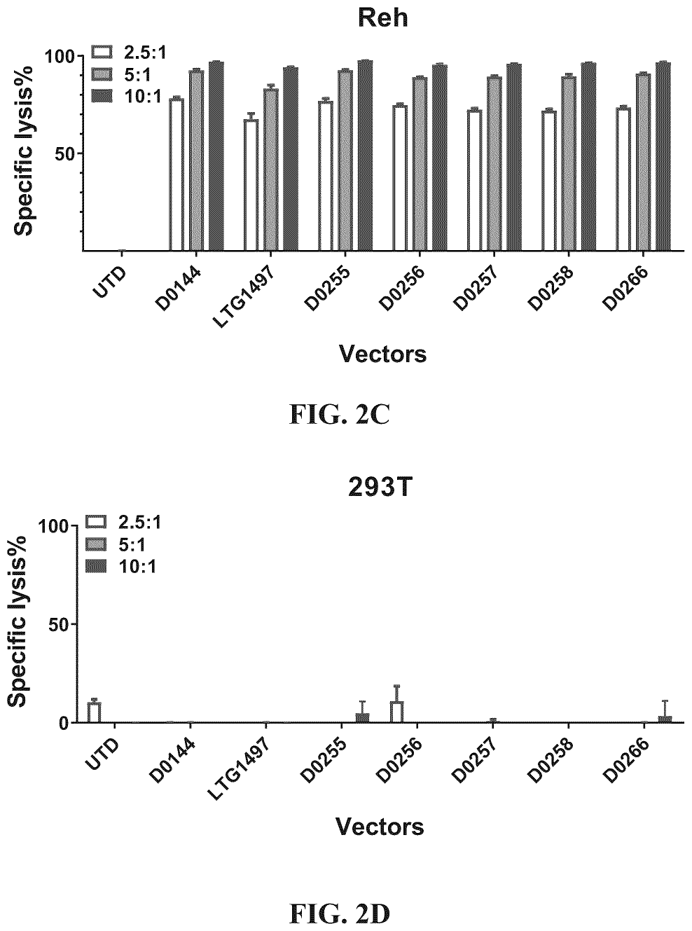

As shown in Example 1 and FIGS. 2A-2D, high cytolytic activity of the fully human CD20/CD19 CARs was demonstrated. Human primary T cells were transduced with LV encoding CAR constructs (D0144, D0255, D0256, D0257, D0258, D0266, and comparator construct LTG1497, see Methods), then incubated for 18 hours with the Raji, NALM-6, REH, or 293T cell lines, stably transduced with firefly luciferase, for luminescence based in vitro killing assays. All leukemia lines tested express CD19 on their surface, while the negative control, 293T do not. CD20 expression varied between tumor lines. The Raji line is CD20 positive, while REH are CD20 negative, as is the control line 293T. NALM-6 line has a weak but detectable expression of CD20.

All tumor lines expressing CD19 and/or CD20 were lysed by CAR T cells, whereas no lysis of the CD19-negative CD20-negative line 293T occurred.

The most potent specific lysis of Raji cells was mediated by CAR constructs D0255 and D0266, as seen by lysis at the lowest effector to target ratio of 2.5:1 (FIG. 2A). Potent and comparable lysis was achieved by all CARs when combined with target lines NALM 6 and Reh (FIG. 2B, 2C).

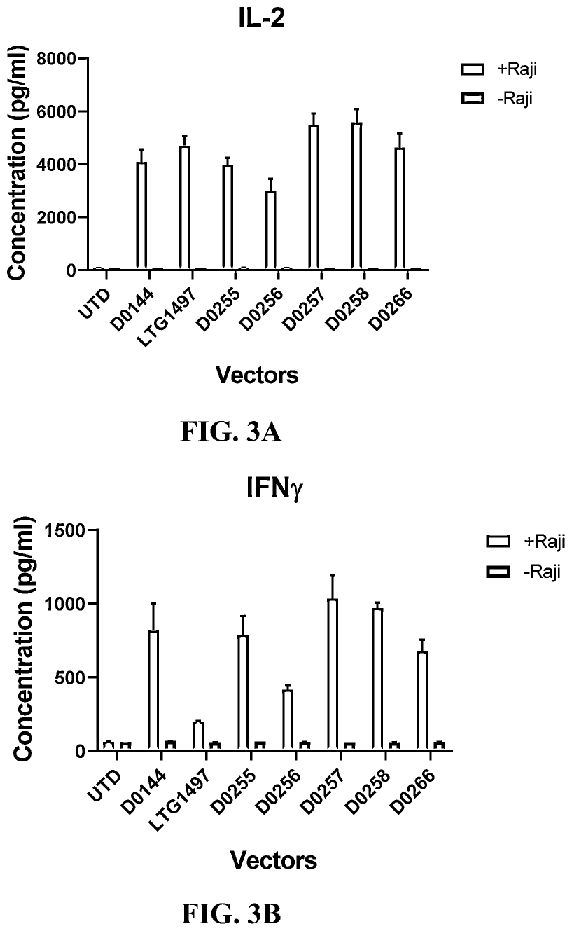

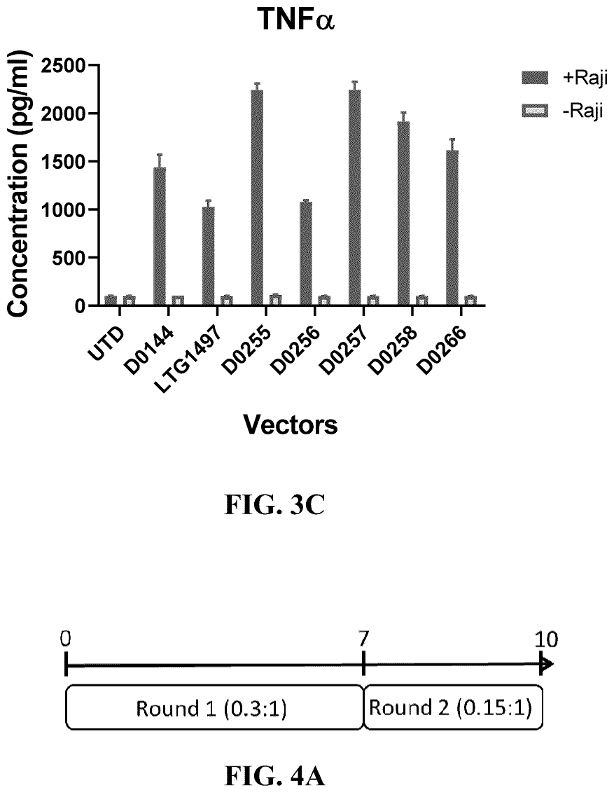

The capacity of anti-CD20/CD19 CAR T cells for cytokine secretion was then evaluated. Tumor cells were co-incubated with CAR T cells or control T cells at effector to target ratio of 10:1 overnight (+ Raji group), and culture supernatants were analyzed by ELISA for IFN gamma, TNF alpha, and IL-2 (c.f., FIGS. 3A-3C). CAR T cells in the absence of targets were included as a negative control (-Raji group). Untransduced (UTD) negative T cell control was also included. All CAR T groups induced cytokines in response to tumor cells, whereas the negative control, -Raji, yielded no appreciable cytokine induction. All CARs strongly induced IFN gamma, TNF alpha, and IL-2 as compared to negative controls.

Th greatest levels of TNF-alpha were elaborated by CARs D0255, D0257, D0258, D0266, and the production of IFN-gamma and IL-2 followed a similar pattern (FIG. 3A-3C).

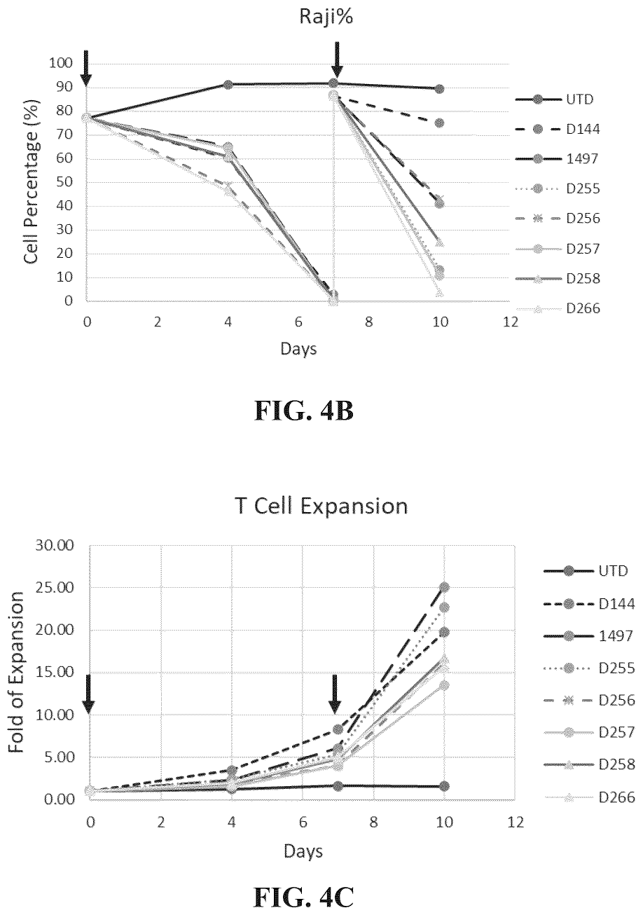

To determine the performance of the fully human Cd20/CD19 CRA candidates in a course of prolonged exposure to antigen, we performed a co-culture experiment wit Raji tumor cells. CAR T cells were placed in culture with Raji lymphoma cells at a low effector to target ratio of 0.3:1, and after 7 days, CAR T cells were restimulated again with Raji cells at the effector to target ratio of 0.15:1, up to study day 10 (FIG. 4A). This setting created a highly challenging environment for CAR T cell function, in order to pinpoint even the smallest functional differences between CAR T candidate constructs. Cell cultures were acquired by flow cytometry on days 0, 7, and 10 to evaluate the live target cell percentage remaining at the end of each co-incubation cycle, and CAR T cell expansion (FIG. 4B-4C). The three CAR T cells that mediated the greatest reduction in Raji target cell; levels at the end of the second co-incubation cycle were D0255, D0257 and D0266 (FIG. 4B). CAR T cells with the best expansion were LTG1497, D0255, and D0144 (FIG. 4C). Therefore we have identified a number of CAR T cell candidates with potent cytotoxicity and expansion properties, as compared to the other constructs in this set and LTG1497 comparator.

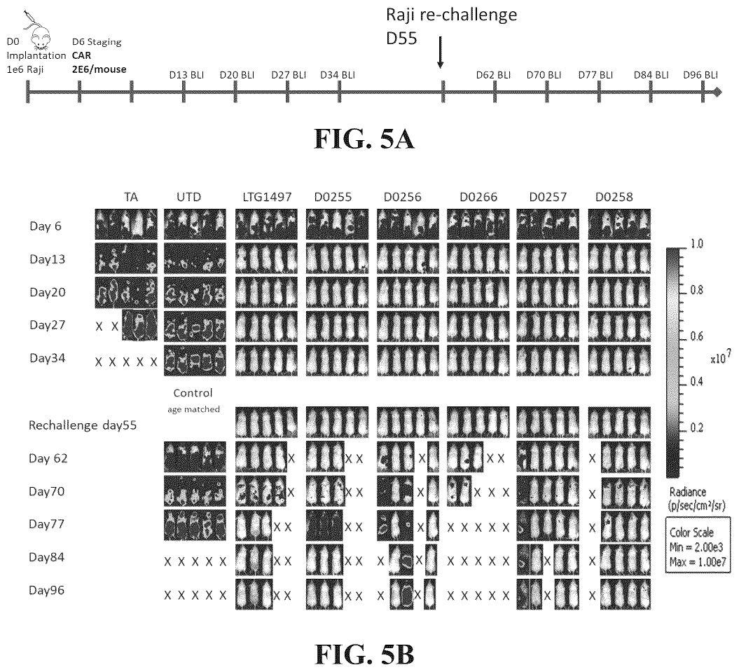

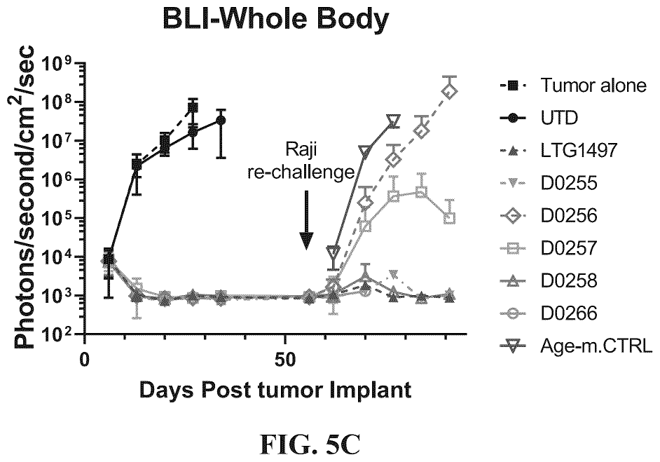

To characterize the functional properties of CAR T cells in vivo, we treated NSG mice with established Raji xenograft tumors with a low dose of two million of fully human CD20/CD19 CAR T cells D0255, D0256, D0266, D0257 or D0258, or the murine scFv CD20/CD19 CAR comparator LTG1497 (FIG. 5A). Tumors were rejected in mice treated with all CAR t constructs by day 13, and mice remained in remission to day 55 (FIG. 5B). By contrast, mice in the control groups tumor alone and UTD developed high tumor burden (FIG. 5B). On day 55, mice in all groups were rechallenged with one million additional Raji cells, and no additional CAR T treatment was administered. One mouse in each group LTG1497, D0256, D0258, and two mice in groups each D0255 and D0266 did not meet rechallenge criteria and were not enrolled. A new, age-matched cohort of mice was also inoculated with Raji cells as a control at this time. Tumor progression was followed up to day 96. On day 96, all enrolled mice were alive and tumor free in CAR groups D0255 and D0258, one mouse was lost in groups each D0257, D0256, LTG1497, and three mice were lost in group D0266. Therefore the ability to clear initial xenograft tumors in vivo was demonstrated for all CAR constructs, and the persistence of CAR T cell anti-tumor function upon tumor rechallenge was demonstrated for constructs D0255, D0258, and LTG1497 (FIG. 5C). The emerging ranking of CAR T candidates in regards to their anti-tumor utility therefore had to be determined empirically and could not have been predicted solely based on CAR architecture or targeting domain composition.

Without being intended to limit to any particular mechanism of action, it is believed that possible reasons for the enhanced therapeutic function associated with the exemplary CARs of the invention include, for example, and not by way of limitation, a) improved lateral movement within the plasma membrane allowing for more efficient signal transduction, b) superior location within plasma membrane microdomains, such as lipid rafts, and greater ability to interact with transmembrane signaling cascades associated with T cell activation, c) superior location within the plasma membrane by preferential movement away from dampening or down-modulatory interactions, such as less proximity to or interaction with phosphatases such as CD45, and d) superior assembly into T cell receptor signaling complexes (i.e. the immune synapse), or any combination thereof.

While the disclosure has been illustrated with an exemplary extracellular fully human CD20/CD19 variable heavy chain only and ScFv antigen binding domains, other nucleotide and/or amino acid variants within the fully human CD20/CD19 variable heavy chain only and ScFv antigen binding domains may be used to derive the fully human CD20/CD19 antigen binding domains for use in the CARs described herein.

Depending on the desired antigen to be targeted, the CAR can be additionally engineered to include the appropriate antigen binding domain that is specific to the desired antigen target. For example, if fully human CD20/CD19 is the desired antigen that is to be targeted, an antibody for fully human CD20/CD19 can be used as the antigen bind domain incorporation into the CAR.

In one aspect of the present invention, there is provided a CAR capable of binding to a non-TSA or non-TAA including, for example and not by way of limitation, an antigen derived from Retroviridae (e.g. human immunodeficiency viruses such as HIV-1 and HIV-LP), Picornaviridae (e.g. poliovirus, hepatitis A virus, enterovirus, human coxsackievirus, rhinovirus, and echovirus), rubella virus, coronavirus, vesicular stomatitis virus, rabies virus, ebola virus, parainfluenza virus, mumps virus, measles virus, respiratory syncytial virus, influenza virus, hepatitis B virus, parvovirus, Adenoviridae, Herpesviridae [e.g. type 1 and type 2 herpes simplex virus (HSV), varicella-zoster virus, cytomegalovirus (CMV), and herpes virus], Poxviridae (e.g. smallpox virus, vaccinia virus, and pox virus), or hepatitis C virus, or any combination thereof.

In another aspect of the present invention, there is provided a CAR capable of binding to an antigen derived from a bacterial strain of Staphylococci, Streptococcus, Escherichia coli, Pseudomonas, or Salmonella. Particularly, there is provided a CAR capable of binding to an antigen derived from an infectious bacterium, for example, Helicobacter pyloris, Legionella pneumophilia, a bacterial strain of Mycobacteria sps. (e.g. M. tuberculosis, M. avium, M. intracellulare, M. kansaii, or M. gordonea), Staphylococcus aureus, Neisseria gonorrhoeae, Neisseria meningitides, Listeria monocytogenes, Streptococcus pyogenes, Group A Streptococcus, Group B Streptococcus (Streptococcus agalactiae), Streptococcus pneumoniae, or Clostridium tetani, or a combination thereof.

### 2. Transmembrane Domain

With respect to the transmembrane domain, the CAR comprises one or more transmembrane domains fused to the extracellular CD19/CD20 antigen binding domain of the CAR.

The transmembrane domain may be derived either from a natural or from a synthetic source. Where the source is natural, the domain may be derived from any membrane-bound or transmembrane protein.

Transmembrane regions of particular use in the CARs described herein may be derived from (i.e. comprise at least the transmembrane region(s) of) the alpha, beta or zeta chain of the T-cell receptor, CD28, CD3 epsilon, CD45, CD4, CD5, CD8, CD9, CD16, CD22, CD28, mesothelin, CD33, CD37, CD64, CD80, CD83, CD86, CD134, CD137, CD154, TNFRSF16, or TNFRSF19. Alternatively the transmembrane domain may be synthetic, in which case it will comprise predominantly hydrophobic residues such as leucine and valine. Preferably a triplet of phenylalanine, tryptophan and valine will be found at each end of a synthetic transmembrane domain. Optionally, a short oligo- or polypeptide linker, preferably between 2 and 10 amino acids in length may form the linkage between the transmembrane domain and the cytoplasmic signaling domain of the CAR. A glycine-serine doublet provides a particularly suitable linker.

In one embodiment, the transmembrane domain that naturally is associated with one of the domains in the CAR is used in addition to the transmembrane domains described supra.

In some instances, the transmembrane domain can be selected or by amino acid substitution to avoid binding of such domains to the transmembrane domains of the same or different surface membrane proteins to minimize interactions with other members of the receptor complex.

In one embodiment, the transmembrane domain in the CAR of the invention is the CD8 transmembrane domain. In one embodiment, the CD8 transmembrane domain comprises the nucleic acid sequence of SEQ ID NO: 35. In one embodiment, the CD8 transmembrane domain comprises the nucleic acid sequence that encodes the amino acid sequence of SEQ ID NO: 36. In another embodiment, the CD8 transmembrane domain comprises the amino acid sequence of SEQ ID NO: 36.

In one embodiment, the encoded transmembrane domain comprises an amino acid sequence having at least one, two or three modifications (e.g., substitutions) but not more than 20, 10 or 5 modifications (e.g., substitutions) of an amino acid sequence of SEQ ID NO: 34, 36, or 48, or a sequence with 95-99% identity to an amino acid sequence of SEQ ID NO: 34, 36, or 48.

In some instances, the transmembrane domain of the CAR comprises the CD8.alpha.hinge domain. In one embodiment, the CD8 hinge domain comprises the nucleic acid sequence of SEQ ID NO: 37. In one embodiment, the CD8 hinge domain comprises the nucleic acid sequence that encodes the amino acid sequence of SEQ ID NO: 38. In another embodiment, the CD8 hinge domain comprises the amino acid sequence of SEQ ID NO: 38, or a sequence with 95-99% identify thereof.

In one embodiment, an isolated nucleic acid molecule is provided wherein the encoded linker domain is derived from the extracellular domain of CD8, and is linked to the transmembrane CD8 domain, the transmembrane CD28 domain, or a combination thereof.

In one embodiment, the transmembrane domain in the CAR of the invention is the CD28 transmembrane domain. In one embodiment, the CD28 transmembrane domain comprises the nucleic acid sequence of SEQ ID NO: 47. In one embodiment, the CD28 transmembrane domain comprises the nucleic acid sequence that encodes the amino acid sequence of SEQ ID NO: 48. In another embodiment, the CD28 transmembrane domain comprises the amino acid sequence of SEQ ID NO: 48.

In one embodiment, the encoded transmembrane domain comprises an amino acid sequence having at least one, two or three modifications (e.g., substitutions) but not more than 20, 10 or 5 modifications (e.g., substitutions) of an amino acid sequence of SEQ ID NO: 47, or a sequence with 95-99% identity to an amino acid sequence of SEQ ID NO: 48.

3. Spacer Domain

In the CAR, a spacer domain can be arranged between the extracellular domain and the transmembrane domain, or between the intracellular domain and the transmembrane domain. The spacer domain means any oligopeptide or polypeptide that serves to link the transmembrane domain with the extracellular domain and/or the transmembrane domain with the intracellular domain. The spacer domain comprises up to 300 amino acids, preferably 10 to 100 amino acids, and most preferably 25 to 50 amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,566, 7,498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

The spacer domain preferably has a sequence that promotes binding of a CAR with an antigen and enhances signaling into a cell. Examples of an amino acid that is expected to promote the binding include cysteine, a charged amino acid, and serine and threonine in a potential glycosylation site, and these amino acids can be used as an amino acid constituting the spacer domain.

As the spacer domain, the entire or a part of amino acid numbers 137-206 (SEQ ID NO: 39) which is a hinge region of CD8.alpha. (NCBI RefSeq: NP.sub.--001759.3), amino acid numbers 135 to 195 of CD8.beta. (GenBank: AAA35664.1), amino acid numbers 315 to 396 of CD4 (NCBI RefSeq: NP.sub.--000607.1), or amino acid numbers 137 to 152 of CD28 (NCBI RefSeq: NP.sub.--006130.1) can be used. Also, as the spacer domain, a part of a constant region of an antibody H chain or L chain can be used. Further, the spacer domain may be an artificially synthesized sequence.

In some instances, the transmembrane domain of the CAR comprises the CD28 hinge domain. In one embodiment, the CD28 hinge domain comprises the nucleic acid sequence of SEQ ID NO: 31. In one embodiment, the CD28 hinge domain comprises the nucleic acid sequence that encodes the amino acid sequence of SEQ ID NO: 32. In another embodiment, the CD28 hinge domain comprises the amino acid sequence of SEQ ID NO: 32, or a sequence with 95-99% identify thereof.

In one embodiment, an isolated nucleic acid molecule is provided wherein the encoded linker domain is derived from the extracellular domain of CD28, and is linked to the transmembrane CD28 domain, the transmembrane CD28 domain, or a combination thereof.

Further, in the CAR, a signal peptide sequence can be linked to the N-terminus. The signal peptide sequence exists at the N-terminus of many secretory proteins and membrane proteins, and has a length of 15 to 30 amino acids. Since many of the protein molecules mentioned above as the intracellular domain have signal peptide sequences, the signal peptides can be used as a signal peptide for the CAR. In one embodiment, the signal peptide comprises the amino acid sequence shown in SEQ ID NO:12.

4. Intracellular Domain

The cytoplasmic domain or otherwise the intracellular signaling domain of the CAR is responsible for activation of at least one of the normal effector functions of the immune cell in which the CAR has been placed in. The term “effector function” refers to a specialized function of a cell. Effector function of a T cell, for example, may be cytolytic activity or helper activity including the secretion of cytokines. Thus the term “intracellular signaling domain” refers to the portion of a protein which transduces the effector function signal and directs the cell to perform a specialized function. While usually the entire intracellular signaling domain can be employed, in many cases it is not necessary to use the entire chain. To the extent that a truncated portion of the intracellular signaling domain is used, such truncated portion may be used in place of the intact chain as long as it transduces the effector function signal. The term intracellular signaling domain is thus meant to include any truncated portion of the intracellular signaling domain sufficient to transduce the effector function signal.

Preferred examples of intracellular signaling domains for use in the CAR include the cytoplasmic sequences of the T cell receptor (TCR) and co-receptors that act in concert to initiate signal transduction following antigen receptor engagement, as well as any derivative or variant of these sequences and any synthetic sequence that has the same functional capability.

It is known that signals generated through the TCR alone are insufficient for full activation of the T cell and that a secondary or co-stimulatory signal is also required. Thus, T cell activation can be said to be mediated by two distinct classes of cytoplasmic signaling sequence. those that initiate antigen-dependent primary activation through the TCR (primary cytoplasmic signaling sequences) and those that act in an antigen-independent manner to provide a secondary or co-stimulatory signal (secondary cytoplasmic signaling sequences).

Primary cytoplasmic signaling sequences regulate primary activation of the TCR complex either in a stimulatory way, or in an inhibitory way. Primary cytoplasmic signaling sequences that act in a stimulatory manner may contain signaling motifs which are known as immunoreceptor tyrosine-based activation motifs or ITAMs.

Examples of ITAM containing primary cytoplasmic signaling sequences that are of particular use in the CARS disclosed herein include those derived from TCR zeta (CD3 Zeta), FcR gamma, FcR beta, CD3 gamma, CD3 delta, CD3 epsilon, CD5, CD22, CD79a, CD79b, and CD66d. Specific, non-limiting examples, of the ITAM include peptides having sequences of amino acid numbers 51 to 164 of CD3.zeta. (NCBI RefSeq: NP.sub.-932170.1), amino acid numbers 45 to 86 of Fc.epsilon.RI.gamma. (NCBI RefSeq: NP.sub.-004097.1), amino acid numbers 201 to 244 of Fc.epsilon.RI.beta. (NCBI RefSeq: NP.sub.--000130.1), amino acid numbers 139 to 182 of CD3.gamma. (NCBI RefSeq: NP.sub.-000064.1), amino acid numbers 128 to 171 of CD3.delta. (NCBI RefSeq: NP.sub.--000723.1), amino acid numbers 153 to 207 of CD3.epsilon. (NCBI RefSeq. NP.sub.-000724.1), amino acid numbers 402 to 495 of CD5 (NCBI RefSeq: NP.sub.--055022.2), amino acid numbers 707 to 847 of 0022 (NCBI RefSeq: NP.sub.--001762.2), amino acid numbers 166 to 226 of CD79a (NCBI RefSeq: NP.sub.--001774.1), amino acid numbers 182 to 229 of CD79b (NCBI RefSeq: NP.sub.-000617.1), and amino acid numbers 177 to 252 of CD66d (NCBI RefSeq: NP.sub.-001806.2), and their variants having the same function as these peptides have. The amino acid number based on amino acid sequence information of NCBI RefSeq ID or GenBank described herein is numbered based on the full length of the precursor (comprising a signal peptide sequence etc.) of each protein. In one embodiment, the cytoplasmic signaling molecule in the CAR comprises a cytoplasmic signaling sequence derived from CD3 zeta.

In a preferred embodiment, the intracellular domain of the CAR can be designed to comprise the CD3-zeta signaling domain by itself or combined with any other desired cytoplasmic domain(s) useful in the context of the CAR. For example, the intracellular domain of the CAR can comprise a CD3 zeta chain portion and a costimulatory signaling region. The costimulatory signaling region refers to a portion of the CAR comprising the intracellular domain of a costimulatory molecule. A costimulatory molecule is a cell surface molecule other than an antigen receptor or their ligands that is required for an efficient response of lymphocytes to an antigen. Examples of such costimulatory molecules include CD27, CD28, 4-1BB (CD137), OX40, CD30, CD40, PD-A, ICOS, lymphocyte function-associated antigen-1 (LFA-1), CD2, CD7, LIGHT, NKG2C, B7-H3, and a ligand that specifically binds with CD83, and the like. Specific, non-limiting examples, of such costimulatory molecules include peptides having sequences of amino acid numbers 236 to 351 of CD2 (NCBI RefSeq: NP.sub.-001758.2), amino acid numbers 421 to 458 of CD4 (NCBI RefSeq: NP.sub.--000607.1), amino acid numbers 402 to 495 of CD5 (NCBI RefSeq: NP.sub.--055022.2), amino acid numbers 207 to 235 of CD8.alpha. (NCBI RefSeq: NP.sub.--001759.3), amino acid numbers 196 to 210 of CD83 (GenBank: AAA35664.1), amino acid numbers 181 to 220 of CD28 (NCBI RefSeq: NP.sub.--006130.1), amino acid numbers 214 to 255 of CD137 (4-1BB, NCBI RefSeq: NP.sub.--001552.2), amino acid numbers 241 to 277 of CD134 (OX40, NCBI RefSeq: NP.sub.--003318.1), and amino acid numbers 166 to 199 of ICOS (NCBI RefSeq: NP.sub.-036224.1), and their variants having the same function as these peptides have. Thus, while the disclosure herein is exemplified primarily with 4-1BB as the co-stimulatory signaling element, other costimulatory elements are within the scope of the disclosure.

The cytoplasmic signaling sequences within the cytoplasmic signaling portion of the CAR may be linked to each other in a random or specified order. Optionally, a short oligo- or polypeptide linker, preferably between 2 and 10 amino acids in length may form the linkage. A glycine-serine doublet provides a particularly suitable linker.

In one embodiment, the intracellular domain is designed to comprise the signaling domain of CD3-zeta and the signaling domain of CD28. In another embodiment, the intracellular domain is designed to comprise the signaling domain of CD3-zeta and the signaling domain of 4-1BB. In yet another embodiment, the intracellular domain is designed to comprise the signaling domain of CD3-zeta and the signaling domain of CD28 and 4-1BB.

In one embodiment, the intracellular domain in the CAR is designed to comprise the signaling domain of 4-1BB and the signaling domain of CD3-zeta, wherein the signaling domain of 4-1BB comprises the nucleic acid sequence set forth in SEQ ID NO: 42, 44, or 45 and the signaling domain of CD3-zeta comprises the nucleic acid sequence set forth in SEQ ID NO: 43.

In another embodiment, the intracellular domain in the disclosed CARs is designed to comprise the signaling domain of CD28 and the signaling domain of CD3-zeta, wherein the signaling domain of CD28 comprises the nucleic acid sequence set forth in SEQ ID NO: 21, and the signaling domain of CD3-zeta comprises the nucleic acid sequence set forth in SEQ ID NO: 42, 45, or 46.

In another embodiment, the intracellular domain in the CAR is designed to comprise the joined signaling domain of CD28_4-1BB, and the signaling domain of CD3-zeta, wherein the signaling domain of CD28_4-1BB comprises the nucleic acid sequence set forth in SEQ ID NO: 25 and the signaling domain of CD3-zeta comprises the nucleic acid sequence set forth in SEQ ID NO: 42, 45 or 46.

5. Additional Description of CARs

Also expressly included within the scope of the invention are functional portions of the CARs disclosed herein. The term “functional portion” when used in reference to a CAR refers to any part or fragment of one or more of the CARs disclosed herein, which part or fragment retains the biological activity of the CAR of which it is a part (the parent CAR). Functional portions encompass, for example, those parts of a CAR that retain the ability to recognize target cells, or detect, treat, or prevent a disease, to a similar extent, the same extent, or to a higher extent, as the parent CAR. In reference to the parent CAR, the functional portion can comprise, for instance, about 10/o, 25%, 30%, 50%, 68%, 80%, 90%, 95%, or more, of the parent CAR.

The functional portion can comprise additional amino acids at the amino or carboxy terminus of the portion, or at both termini, which additional amino acids are not found in the amino acid sequence of the parent CAR. Desirably, the additional amino acids do not interfere with the biological function of the functional portion, e.g., recognize target cells, detect cancer, treat or prevent cancer, etc. More desirably, the additional amino acids enhance the biological activity, as compared to the biological activity of the parent CAR.

Included in the scope of the disclosure are functional variants of the CARs disclosed herein. The term “functional variant” as used herein refers to a CAR, polypeptide, or protein having substantial or significant sequence identity or similarity to a parent CAR, which functional variant retains the biological activity of the CAR of which it is a variant. Functional variants encompass, for example, those variants of the CAR described herein (the parent CAR) that retain the ability to recognize target cells to a similar extent, the same extent, or to a higher extent, as the parent CAR. In reference to the parent CAR, the functional variant can, for instance, be at least about 30%, 50%, 75%, 80%, 90%, 98% or more identical in amino acid sequence to the parent CAR.

A functional variant can, for example, comprise the amino acid sequence of the parent CAR with at least one conservative amino acid substitution. Alternatively or additionally, the functional variants can comprise the amino acid sequence of the parent CAR with at least one non-conservative amino acid substitution. In this case, it is preferable for the non-conservative amino acid substitution to not interfere with or inhibit the biological activity of the functional variant. The non-conservative amino acid substitution may enhance the biological activity of the functional variant, such that the biological activity of the functional variant is increased as compared to the parent CAR.

Amino acid substitutions of the CARs are preferably conservative amino acid substitutions. Conservative amino acid substitutions are known in the art, and include amino acid substitutions in which one amino acid having certain physical and/or chemical properties is exchanged for another amino acid that has the same or similar chemical or physical properties. For instance, the conservative amino acid substitution can be an acidic/negatively charged polar amino acid substituted for another acidic/negatively charged polar amino acid (e.g., Asp or Glu), an amino acid with a nonpolar side chain substituted for another amino acid with a nonpolar side chain (e.g., Ala, Gly, Val, He, Leu, Met, Phe, Pro, Trp, Cys, Val, etc.), a basic/positively charged polar amino acid substituted for another basic/positively charged polar amino acid (e.g. Lys, His, Arg, etc.), an uncharged amino acid with a polar side chain substituted for another uncharged amino acid with a polar side chain (e.g., Asn, Gin, Ser, Thr, Tyr, etc.), an amino acid with a beta-branched side-chain substituted for another amino acid with a beta-branched side-chain (e.g., He, Thr, and Val), an amino acid with an aromatic side-chain substituted for another amino acid with an aromatic side chain (e.g., His, Phe, Trp, and Tyr), etc.

The CAR can consist essentially of the specified amino acid sequence or sequences described herein, such that other components, e.g., other amino acids, do not materially change the biological activity of the functional variant.

The CARs (including functional portions and functional variants) can be of any length, i.e., can comprise any number of amino acids, provided that the CARs (or functional portions or functional variants thereof) retain their biological activity, e.g., the ability to specifically bind to antigen, detect diseased cells in a mammal, or treat or prevent disease in a mammal, etc. For example, the CAR can be about 50 to about 5000 amino acids long, such as 50, 70, 75, 100, 125, 150, 175, 200, 300, 400, 500, 600, 700, 800, 900, 1000 or more amino acids in length.

The CARs (including functional portions and functional variants of the invention) can comprise synthetic amino acids in place of one or more naturally-occurring amino acids. Such synthetic amino acids are known in the art, and include, for example, aminocyclohexane carboxylic acid, norleucine, -amino n-decanoic acid, homoserine, S-acetylaminomethyl-cysteine, trans-3- and trans-4-hydroxyproline, 4-aminophenylalanine, 4-nitrophenylalanine, 4-chlorophenylalanine, 4-carboxyphenylalanine, β-phenylserine β-hydroxyphenylalanine, phenylglycine, a-naphthylalanine, cyclohexylalanine, cyclohexylglycine, indoline-2-carboxylic acid, 1,2,3,4-tetrahydroisoquinoline-3-carboxylic acid, aminomalonic acid, aminomalonic acid monoamide, N′-benzyl-N′-methyl-lysine, N,N′-dibenzyl-lysine, β-hydroxylysine, omithine, β-aminocyclopentane carboxylic acid, a-aminocyclohexane carboxylic acid, a-aminocycloheptane carboxylic acid, a-(2-amino-2-norbornane)-carboxylic acid, γ-diaminobutyric acid, β-diaminopropionic acid, homophenylalanine, and a-tert-butylglycine.

The CARs (including functional portions and functional variants) can be glycosylated, amidated, carboxylated, phosphorylated, esterified, N-acylated, cyclized via, e.g., a disulfide bridge, or converted into an acid addition salt and/or optionally dimerized or polymerized, or conjugated.

The CARs (including functional portions and functional variants thereof) can be obtained by methods known in the art. The CARs may be made by any suitable method of making polypeptides or proteins. Suitable methods of de novo synthesizing polypeptides and proteins are described in references, such as Chan et al., Fmoc Solid Phase Peptide Synthesis, Oxford University Press, Oxford, United Kingdom, 2000; Peptide and Protein Drug Analysis, ed. Reid, R., Marcel Dekker, Inc., 2000; Epitope Mapping, ed. Westwood et al., Oxford University Press, Oxford, United Kingdom, 2001; and U.S. Pat. No. 5,449,752. Also, polypeptides and proteins can be recombinantly produced using the nucleic acids described herein using standard recombinant methods. See, for instance, Sambrook et al., Molecular Cloning: A Laboratory Manual, 3rd ed., Cold Spring Harbor Press, Cold Spring Harbor, N Y 2001; and Ausubel et al., Current Protocols in Molecular Biology, Greene Publishing Associates and John Wiley & Sons, NY, 1994. Further, some of the CARs (including functional portions and functional variants thereof) can be isolated and/or purified from a source, such as a plant, a bacterium, an insect, a mammal, e.g., a rat, a human, etc. Methods of isolation and purification are well-known in the art. Alternatively, the CARs described herein (including functional portions and functional variants thereof) can be commercially synthesized by companies. In this respect, the CARs can be synthetic, recombinant, isolated, and/or purified.

B. Antibodies and Antigen Binding Fragments

One embodiment further provides a CAR, a T cell expressing a CAR, an antibody, or antigen binding domain or portion thereof, which specifically binds to one or more of the antigens disclosed herein. As used herein, a “T cell expressing a CAR,” or a “CAR T cell” means a T cell expressing a CAR, and has antigen specificity determined by, for example, the antibody-derived targeting domain of the CAR.

As used herein, and “antigen binding domain” can include an antibody and antigen binding fragments thereof. The term “antibody” is used herein in the broadest sense and encompasses various antibody structures, including but not limited to monoclonal antibodies, polyclonal antibodies, multi-specific antibodies (e.g., bispecific antibodies), and antigen binding fragments thereof, so long as they exhibit the desired antigen-binding activity. Non-limiting examples of antibodies include, for example, intact immunoglobulins and variants and fragments thereof known in the art that retain binding affinity for the antigen.

A “monoclonal antibody” is an antibody obtained from a population of substantially homogeneous antibodies, i.e., the individual antibodies comprising the population are identical except for possible naturally occurring mutations that may be present in minor amounts. Monoclonal antibodies are highly specific, being directed against a single antigenic epitope. The modifier “monoclonal” indicates the character of the antibody as being obtained from a substantially homogeneous population of antibodies, and is not to be construed as requiring production of the antibody by any particular method. In some examples, a monoclonal antibody is an antibody produced by a single clone of B lymphocytes or by a cell into which nucleic acid encoding the light and heavy variable regions of the antibody of a single antibody (or an antigen binding fragment thereof) have been transfected, or a progeny thereof. In some examples monoclonal antibodies are isolated from a subject. Monoclonal antibodies can have conservative amino acid substitutions which have substantially no effect on antigen binding or other immunoglobulin functions. Exemplary methods of production of monoclonal antibodies are known, for example, see Harlow & Lane, Antibodies, A Laboratory Manual, 2nd ed. Cold Spring Harbor Publications, New York (2013).

Typically, an immunoglobulin has heavy (H) chains and light (L) chains interconnected by disulfide bonds. Immunoglobulin genes include the kappa, lambda, alpha, gamma, delta, epsilon and mu constant region genes, as well as the myriad immunoglobulin variable domain genes. There are two types of light chain, lambda (λ) and kappa (κ). There are five main heavy chain classes (or isotypes) which determine the functional activity of an antibody molecule: IgM, IgD, IgG, IgA and IgE.

Each heavy and light chain contains a constant region (or constant domain) and a variable region (or variable domain; see, e.g., Kindt et al. Kuby Immunology, 6^{th} ed., W.H. Freeman and Co., page 91 (2007).) In several embodiments, the heavy and the light chain variable regions combine to specifically bind the antigen. In additional embodiments, only the heavy chain variable region is required. For example, naturally occurring camelid antibodies consisting of a heavy chain only are functional and stable in the absence of light chain (see, e.g., Hamers-Casterman et al., Nature, 363:446-448, 1993; Sheriff et al., Nat. Struct. Biol., 3:733-736, 1996). References to “VH” or “VH” refer to the variable region of an antibody heavy chain, including that of an antigen binding fragment, such as Fv, ScFv, dsFv or Fab. References to “VL” or “VL” refer to the variable domain of an antibody light chain, including that of an Fv, ScFv, dsFv or Fab.

Light and heavy chain variable regions contain a “framework” region interrupted by three hypervariable regions, also called “complementarity-determining regions” or “CDRs” (see, e.g., Kabat et al., Sequences of Proteins of Immunological Interest, U.S. Department of Health and Human Services, 1991). The sequences of the framework regions of different light or heavy chains are relatively conserved within a species. The framework region of an antibody, that is the combined framework regions of the constituent light and heavy chains, serves to position and align the CDRs in three-dimensional space.

The CDRs are primarily responsible for binding to an epitope of an antigen. The amino acid sequence boundaries of a given CDR can be readily determined using any of a number of well-known schemes, including those described by Kabat el al. (“Sequences of Proteins of Immunological Interest,” 5^{th} Ed. Public Health Service, National Institutes of Health, Bethesda, M D, 1991; “Kabat” numbering scheme), Al-Lazikani et al., (JMB 273,927-948, 1997; “Chothia” numbering scheme), and Lefranc et al. (“IMGT unique numbering for immunoglobulin and T cell receptor variable domains and Ig superfamily V-like domains,” Dev. Comp. Immunol., 27:55-77, 2003; “IMGT” numbering scheme). The CDRs of each chain are typically referred to as CDR1, CDR2, and CDR3 (from the N-terminus to C-terminus), and are also typically identified by the chain in which the particular CDR is located. Thus, a VH CDR3 is the CDR3 from the variable domain of the heavy chain of the antibody in which it is found, whereas a VL CDR1 is the CDR1 from the variable domain of the light chain of the antibody in which it is found. Light chain CDRs are sometimes referred to as LCDR1, LCDR2, and LCDR3. Heavy chain CDRs are sometimes referred to as LCDR1, LCDR2, and LCDR3.

An “antigen binding fragment” is a portion of a full length antibody that retains the ability to specifically recognize the cognate antigen, as well as various combinations of such portions. Non-limiting examples of antigen binding fragments include Fv, Fab, Fab′, Fab′-SH, F(ab′)2; diabodies; linear antibodies; single-chain antibody molecules (e.g. ScFv); and multi-specific antibodies formed from antibody fragments. Antibody fragments include antigen binding fragments either produced by the modification of whole antibodies or those synthesized de novo using recombinant DNA methodologies (see, e.g., Kontermann and Dubel (Ed), Antibody Engineering, Vols. 1-2, 2nd Ed., Springer Press, 2010).

A single-chain antibody (ScFv) is a genetically engineered molecule containing the VH and VL domains of one or more antibody(ies) linked by a suitable polypeptide linker as a genetically fused single chain molecule (see, for example, Bird et al., Science, 242:423 426, 1988; Huston et al., Proc. Natl. Acad. Sci., 85:5879 5883, 1988; Ahmad et al., Clin. Dev. Immunol., 2012, doi:10.1155/2012/980250; Marbry, IDrugs, 13:543-549, 2010). The intramolecular orientation of the VH-domain and the VL-domain in a ScFv, is typically not decisive for ScFvs. Thus, ScFvs with both possible arrangements (VH-domain-linker domain-VL-domain; VL-domain-linker domain-VH-domain) may be used.

In a dsFv, the heavy and light chain variable chains have been mutated to introduce a disulfide bond to stabilize the association of the chains. Diabodies also are included, which are bivalent, bispecific antibodies in which VH and VL domains are expressed on a single polypeptide chain, but using a linker that is too short to allow for pairing between the two domains on the same chain, thereby forcing the domains to pair with complementary domains of another chain and creating two antigen binding sites (see, for example, Holliger et al., Proc. Natl. Acad. Sci., 90:6444 6448, 1993; Poljak et al., Structure, 2:1121 1123, 1994).

Antibodies also include genetically engineered forms such as chimeric antibodies (such as humanized murine antibodies) and heteroconjugate antibodies (such as bispecific antibodies). See also, Pierce Catalog and Handbook, 1994-1995 (Pierce Chemical Co., Rockford, IL); Kuby, J., Immunology, 3rd Ed., W.H. Freeman & Co., New York, 1997.

Non-naturally occurring antibodies can be constructed using solid phase peptide synthesis, can be produced recombinantly, or can be obtained, for example, by screening combinatorial libraries consisting of variable heavy chains and variable light chains as described by Huse et al., Science 246:1275-1281 (1989), which is incorporated herein by reference. These and other methods of making, for example, chimeric, humanized, CDR-grafted, single chain, and bifunctional antibodies, are well known to those skilled in the art (Winter and Harris, Immunol. Today 14:243-246 (1993); Ward et al., Nature 341:544-546 (1989); Harlow and Lane, supra, 1988; Hilyard et al., Protein Engineering: A practical approach (IRL Press 1992); Borrabeck, Antibody Engineering, 2d ed. (Oxford University Press 1995); each of which is incorporated herein by reference).

An “antibody that binds to the same epitope” as a reference antibody refers to an antibody that blocks binding of the reference antibody to its antigen in a competition assay by 50% or more, and conversely, the reference antibody blocks binding of the antibody to its antigen in a competition assay by 50% or more. Antibody competition assays are known, and an exemplary competition assay is provided herein.

A “humanized” antibody or antigen binding fragment includes a human framework region and one or more CDRs from a non-human (such as a mouse, rat, or synthetic) antibody or antigen binding fragment. The non-human antibody or antigen binding fragment providing the CDRs is termed a “donor,” and the human antibody or antigen binding fragment providing the framework is termed an “acceptor.” In one embodiment, all the CDRs are from the donor immunoglobulin in a humanized immunoglobulin. Constant regions need not be present, but if they are, they can be substantially identical to human immunoglobulin constant regions, such as at least about 85-90%, such as about 95% or more identical. Hence, all parts of a humanized antibody or antigen binding fragment, except possibly the CDRs, are substantially identical to corresponding parts of natural human antibody sequences.

A “chimeric antibody” is an antibody which includes sequences derived from two different antibodies, which typically are of different species. In some examples, a chimeric antibody includes one or more CDRs and/or framework regions from one human antibody and CDRs and/or framework regions from another human antibody.

A “fully human antibody” or “human antibody” is an antibody which includes sequences from (or derived from) the human genome, and does not include sequence from another species. In some embodiments, a human antibody includes CDRs, framework regions, and (if present) an Fc region from (or derived from) the human genome. Human antibodies can be identified and isolated using technologies for creating antibodies based on sequences derived from the human genome, for example by phage display or using transgenic animals (see, e.g., Barbas et al. Phage display: A Laboratory Manuel. 1st Ed. New York: Cold Spring Harbor Laboratory Press, 2004. Print.; Lonberg, Nat. Biotech., 23: 1117-1125, 2005; Lonenberg, Curr. Opin. Immunol., 20:450-459, 2008).

An antibody may have one or more binding sites. If there is more than one binding site, the binding sites may be identical to one another or may be different. For instance, a naturally-occurring immunoglobulin has two identical binding sites, a single-chain antibody or Fab fragment has one binding site, while a bispecific or bifunctional antibody has two different binding sites.

Methods of testing antibodies for the ability to bind to any functional portion of the CAR are known in the art and include any antibody-antigen binding assay, such as, for example, radioimmunoassay (RIA), ELISA, Western blot, immunoprecipitation, and competitive inhibition assays (see, e.g., Janeway et al., infra, U.S. Patent Application Publication No. 2002/0197266 A1, and U.S. Pat. No. 7,338,929).

Also, a CAR, a T cell expressing a CAR, an antibody, or antigen binding portion thereof, can be modified to comprise a detectable label, such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles).

C. Conjugates

A CAR, a T cell expressing a CAR, or monoclonal antibodies, or antigen binding fragments thereof, specific for one or more of the antigens disclosed herein, can be conjugated to an agent, such as an effector molecule or detectable marker, using any number of means known to those of skill in the art. Both covalent and noncovalent attachment means may be used. Conjugates include, but are not limited to, molecules in which there is a covalent linkage of an effector molecule or a detectable marker to an antibody or antigen binding fragment that specifically binds one or more of the antigens disclosed herein. One of skill in the art will appreciate that various effector molecules and detectable markers can be used, including (but not limited to) chemotherapeutic agents, anti-angiogenic agents, toxins, radioactive agents such as ¹²⁵I, ³²P, ¹⁴C, ³H and ³⁵S and other labels, target moieties and ligands, etc.

The choice of a particular effector molecule or detectable marker depends on the particular target molecule or cell, and the desired biological effect. Thus, for example, the effector molecule can be a cytotoxin that is used to bring about the death of a particular target cell (such as a tumor cell).

The procedure for attaching an effector molecule or detectable marker to an antibody or antigen binding fragment varies according to the chemical structure of the effector. Polypeptides typically contain a variety of functional groups; such as carboxylic acid (COOH), free amine (—NH₂) or sulfhydryl (—SH) groups, which are available for reaction with a suitable functional group on an antibody to result in the binding of the effector molecule or detectable marker. Alternatively, the antibody or antigen binding fragment is derivatized to expose or attach additional reactive functional groups. The derivatization may involve attachment of any of a number of known linker molecules such as those available from Pierce Chemical Company, Rockford, IL. The linker can be any molecule used to join the antibody or antigen binding fragment to the effector molecule or detectable marker. The linker is capable of forming covalent bonds to both the antibody or antigen binding fragment and to the effector molecule or detectable marker. Suitable linkers are well known to those of skill in the art and include, but are not limited to, straight or branched-chain carbon linkers, heterocyclic carbon linkers, or peptide linkers. Where the antibody or antigen binding fragment and the effector molecule or detectable marker are polypeptides, the linkers may be joined to the constituent amino acids through their side groups (such as through a disulfide linkage to cysteine) or to the alpha carbon amino and carboxyl groups of the terminal amino acids.

In several embodiments, the linker can include a spacer element, which, when present, increases the size of the linker such that the distance between the effector molecule or the detectable marker and the antibody or antigen binding fragment is increased. Exemplary spacers are known to the person of ordinary skill, and include those listed in U.S. Pat. Nos. 7,964,566, 7,498,298, 6,884,869, 6,323,315, 6,239,104, 6,034,065, 5,780,588, 5,665,860, 5,663,149, 5,635,483, 5,599,902, 5,554,725, 5,530,097, 5,521,284, 5,504,191, 5,410,024, 5,138,036, 5,076,973, 4,986,988, 4,978,744, 4,879,278, 4,816,444, and 4,486,414, as well as U.S. Pat. Pub. Nos. 20110212088 and 20110070248, each of which is incorporated by reference herein in its entirety.

In some embodiments, the linker is cleavable under intracellular conditions, such that cleavage of the linker releases the effector molecule or detectable marker from the antibody or antigen binding fragment in the intracellular environment. In yet other embodiments, the linker is not cleavable and the effector molecule or detectable marker is released, for example, by antibody degradation. In some embodiments, the linker is cleavable by a cleaving agent that is present in the intracellular environment (for example, within a lysosome or endosome or caveolea). The linker can be, for example, a peptide linker that is cleaved by an intracellular peptidase or protease enzyme, including, but not limited to, a lysosomal or endosomal protease. In some embodiments, the peptide linker is at least two amino acids long or at least three amino acids long. However, the linker can be 4, 5, 6, 7, 8, 9, 10, 11, 12, 13, 14 or 15 amino acids long, such as 1-2, 1-3, 2-5, 3-10, 3-15, 1-5, 1-10, 1-15 amino acids long. Proteases can include cathepsins B and D and plasmin, all of which are known to hydrolyze dipeptide drug derivatives resulting in the release of active drug inside target cells (see, for example, Dubowchik and Walker, 1999, Pharm. Therapeutics 83:67-123). For example, a peptide linker that is cleavable by the thiol-dependent protease cathepsin-B, can be used (for example, a Phenylalanine-Leucine or a Glycine-Phenylalanine-Leucine-Glycine linker). Other examples of such linkers are described, for example, in U.S. Pat. No. 6,214,345, incorporated herein by reference. In a specific embodiment, the peptide linker cleavable by an intracellular protease is a Valine-Citruline linker or a Phenylalanine-Lysine linker (see, for example, U.S. Pat. No. 6,214,345, which describes the synthesis of doxorubicin with the Valine-Citruline linker).

In other embodiments, the cleavable linker is pH-sensitive, i.e., sensitive to hydrolysis at certain pH values. Typically, the pH-sensitive linker is hydrolyzable under acidic conditions. For example, an acid-labile linker that is hydrolyzable in the lysosome (for example, a hydrazone, semicarbazone, thiosemicarbazone, cis-aconitic amide, orthoester, acetal, ketal, or the like) can be used. (See, for example, U.S. Pat. Nos. 5,122,368; 5,824,805; 5,622,929; Dubowchik and Walker, 1999, Pharm. Therapeutics 83:67-123; Neville et al., 1989, Biol. Chem. 264:14653-14661.) Such linkers are relatively stable under neutral pH conditions, such as those in the blood, but are unstable at below pH 5.5 or 5.0, the approximate pH of the lysosome. In certain embodiments, the hydrolyzable linker is a thioether linker (such as, for example, a thioether attached to the therapeutic agent via an acylhydrazone bond (see, for example, U.S. Pat. No. 5,622,929).

In other embodiments, the linker is cleavable under reducing conditions (for example, a disulfide linker). A variety of disulfide linkers are known in the art, including, for example, those that can be formed using SATA (N-succinimidyl-S-acetylthioacetate), SPDP (N-succinimidyl-3-(2-pyridyldithio)propionate), SPDB (N-succinimidyl-3-(2-pyridyldithio)butyrate) and SMPT (N-succinimidyl-oxycarbonyl-alpha-methyl-alpha-(2-pyridyl-dithio)toluene)-, SPDB and SMPT. (See, for example, Thorpe et al., 1987, Cancer Res. 47:5924-5931; Wawrzynczak et al., In Immunoconjugates: Antibody Conjugates in Radioimagery and Therapy of Cancer (C. W. Vogel ed., Oxford U. Press, 1987); Phillips et al., Cancer Res. 68:92809290, 2008). See also U.S. Pat. No. 4,880,935.)

In yet other specific embodiments, the linker is a malonate linker (Johnson et al., 1995, Anticancer Res. 15:1387-93), a maleimidobenzoyl linker (Lau et al., 1995, Bioorg-Med-Chem. 3(10):1299-1304), or a 3′-N-amide analog (Lau et al., 1995, Bioorg-Med-Chem. 3(10):1305-12).

In yet other embodiments, the linker is not cleavable and the effector molecule or detectable marker is released by antibody degradation. (See U.S. Publication No. 2005/0238649 incorporated by reference herein in its entirety).

In several embodiments, the linker is resistant to cleavage in an extracellular environment. For example, no more than about 20%, no more than about 15%, no more than about 10%, no more than about 5%, no more than about 3%, or no more than about 1% of the linkers, in a sample of conjugate, are cleaved when the conjugate is present in an extracellular environment (for example, in plasma). Whether or not a linker is resistant to cleavage in an extracellular environment can be determined, for example, by incubating the conjugate containing the linker of interest with plasma for a predetermined time period (for example, 2, 4, 8, 16, or 24 hours) and then quantitating the amount of free effector molecule or detectable marker present in the plasma. A variety of exemplary linkers that can be used in conjugates are described in WO 2004-010957, U.S. Publication No. 2006/0074008, U.S. Publication No. 20050238649, and U.S. Publication No. 2006/0024317, each of which is incorporated by reference herein in its entirety.

In several embodiments, conjugates of a CAR, a T cell expressing a CAR, an antibody, or antigen binding portion thereof, and one or more small molecule toxins, such as a calicheamicin, maytansinoids, dolastatins, auristatins, a trichothecene, and CC1065, and the derivatives of these toxins that have toxin activity, are provided.

Maytansine compounds suitable for use as maytansinoid toxin moieties are well known in the art, and can be isolated from natural sources according to known methods, produced using genetic engineering techniques (see Yu et al (2002) PNAS 99:7968-7973), or maytansinol and maytansinol analogues prepared synthetically according to known methods. Maytansinoids are mitototic inhibitors which act by inhibiting tubulin polymerization. Maytansine was first isolated from the east African shrub Maytenus serrata (U.S. Pat. No. 3,896,111). Subsequently, it was discovered that certain microbes also produce maytansinoids, such as maytansinol and C-3 maytansinol esters (U.S. Pat. No. 4,151,042). Synthetic maytansinol and derivatives and analogues thereof are disclosed, for example, in U.S. Pat. Nos. 4,137,230; 4,248,870; 4,256,746; 4,260,608; 4,265,814; 4,294,757; 4,307,016; 4,308,268; 4,308,269; 4,309,428; 4,313,946; 4,315,929; 4,317,821; 4,322,348; 4,331,598; 4,361,650; 4,364,866; 4,424,219; 4,450,254; 4,362,663; and 4,371,533, each of which is incorporated herein by reference. Conjugates containing maytansinoids, methods of making same, and their therapeutic use are disclosed, for example, in U.S. Pat. Nos. 5,208,020; 5,416,064; 6,441,163 and European Patent EP 0 425 235 B1, the disclosures of which are hereby expressly incorporated by reference.

Additional toxins can be employed with a CAR, a T cell expressing a CAR, an antibody, or antigen binding portion thereof. Exemplary toxins include Pseudomonas exotoxin (PE), ricin, abrin, diphtheria toxin and subunits thereof, ribotoxin, ribonuclease, saporin, and calicheamicin, as well as botulinum toxins A through F. These toxins are well known in the art and many are readily available from commercial sources (for example, Sigma Chemical Company, St. Louis, MO). Contemplated toxins also include variants of the toxins (see, for example, see, U.S. Pat. Nos. 5,079,163 and 4,689,401).

Saporin is a toxin derived from Saponaria officinalis that disrupts protein synthesis by inactivating the 60S portion of the ribosomal complex (Stirpe et al., Bio/Technology, 10:405-412, 1992). However, the toxin has no mechanism for specific entry into cells, and therefore requires conjugation to an antibody or antigen binding fragment that recognizes a cell-surface protein that is internalized in order to be efficiently taken up by cells.

Diphtheria toxin is isolated from Corynebacterium diphtheriae. Typically, diphtheria toxin for use in immunotoxins is mutated to reduce or to eliminate non-specific toxicity. A mutant known as CRM107, which has full enzymatic activity but markedly reduced non-specific toxicity, has been known since the 1970's (Laird and Groman, J. Virol. 19:220, 1976), and has been used in human clinical trials. See, U.S. Pat. Nos. 5,792,458 and 5,208,021.

Ricin is the lectin RCA60 from Ricinus communis (Castor bean). For examples of ricin, see, U.S. Pat. Nos. 5,079,163 and 4,689,401. Ricinus communis agglutinin (RCA) occurs in two forms designated RCA₆₀ and RCA₁₂₀ according to their molecular weights of approximately 65 and 120 kD, respectively (Nicholson & Blaustein, J. Biochim. Biophys. Acta 266:543, 1972). The A chain is responsible for inactivating protein synthesis and killing cells. The B chain binds ricin to cell-surface galactose residues and facilitates transport of the A chain into the cytosol (Olsnes et al., Nature 249:627-631, 1974 and U.S. Pat. No. 3,060,165).

Ribonucleases have also been conjugated to targeting molecules for use as immunotoxins (see Suzuki et al., Nat. Biotech. 17:265-70, 1999). Exemplary ribotoxins such as α-sarcin and restrictocin are discussed in, for example Rathore et al., Gene 190:31-5, 1997; and Goyal and Batra, Biochem. 345 Pt 2:247-54, 2000. Calicheamicins were first isolated from Micromonospora echinospora and are members of the enediyne antitumor antibiotic family that cause double strand breaks in DNA that lead to apoptosis (see, for example Lee et al., J. Antibiot. 42:1070-87,1989). The drug is the toxic moiety of an immunotoxin in clinical trials (see, for example, Gillespie et al., Ann. Oncol. 11:735-41, 2000).

Abrin includes toxic lectins from Abrus precatorius. The toxic principles, abrin a, b, c, and d, have a molecular weight of from about 63 and 67 kD and are composed of two disulfide-linked polypeptide chains A and B. The A chain inhibits protein synthesis; the B chain (abrin-b) binds to D-galactose residues (see, Funatsu et al., Agr. Biol. Chem. 52:1095, 1988; and Olsnes, Methods Enzymol. 50:330-335, 1978).

A CAR, a T cell expressing a CAR, monoclonal antibodies, antigen binding fragments thereof, specific for one or more of the antigens disclosed herein, can also be conjugated with a detectable marker; for example, a detectable marker capable of detection by ELISA, spectrophotometry, flow cytometry, microscopy or diagnostic imaging techniques (such as computed tomography (CT), computed axial tomography (CAT) scans, magnetic resonance imaging (MRI), nuclear magnetic resonance imaging NMRI), magnetic resonance tomography (MTR), ultrasound, fiberoptic examination, and laparoscopic examination). Specific, non-limiting examples of detectable markers include fluorophores, chemiluminescent agents, enzymatic linkages, radioactive isotopes and heavy metals or compounds (for example super paramagnetic iron oxide nanocrystals for detection by MRI). For example, useful detectable markers include fluorescent compounds, including fluorescein, fluorescein isothiocyanate, rhodamine, 5-dimethylamine-1-napthalenesulfonyl chloride, phycoerythrin, lanthanide phosphors and the like. Bioluminescent markers are also of use, such as luciferase, Green fluorescent protein (GFP), Yellow fluorescent protein (YFP). A CAR, a T cell expressing a CAR, an antibody, or antigen binding portion thereof, can also be conjugated with enzymes that are useful for detection, such as horseradish peroxidase, β-galactosidase, luciferase, alkaline phosphatase, glucose oxidase and the like. When a CAR, a T cell expressing a CAR, an antibody, or antigen binding portion thereof, is conjugated with a detectable enzyme, it can be detected by adding additional reagents that the enzyme uses to produce a reaction product that can be discerned. For example, when the agent horseradish peroxidase is present the addition of hydrogen peroxide and diaminobenzidine leads to a colored reaction product, which is visually detectable. A CAR, a T cell expressing a CAR, an antibody, or antigen binding portion thereof, may also be conjugated with biotin, and detected through indirect measurement of avidin or streptavidin binding. It should be noted that the avidin itself can be conjugated with an enzyme or a fluorescent label.

A CAR, a T cell expressing a CAR, an antibody, or antigen binding portion thereof, may be conjugated with a paramagnetic agent, such as gadolinium. Paramagnetic agents such as superparamagnetic iron oxide are also of use as labels. Antibodies can also be conjugated with lanthanides (such as europium and dysprosium), and manganese. An antibody or antigen binding fragment may also be labeled with a predetermined polypeptide epitopes recognized by a secondary reporter (such as leucine zipper pair sequences, binding sites for secondary antibodies, metal binding domains, epitope tags).

A CAR, a T cell expressing a CAR, an antibody, or antigen binding portion thereof, can also be conjugated with a radiolabeled amino acid. The radiolabel may be used for both diagnostic and therapeutic purposes. For instance, the radiolabel may be used to detect one or more of the antigens disclosed herein and antigen expressing cells by x-ray, emission spectra, or other diagnostic techniques. Further, the radiolabel may be used therapeutically as a toxin for treatment of tumors in a subject, for example for treatment of a neuroblastoma. Examples of labels for polypeptides include, but are not limited to, the following radioisotopes or radionucleotides: ³H, ¹⁴C, ¹⁵N, ³⁵S, ⁹⁰Y, ⁹⁹Tc, ¹¹¹Min, ¹²⁵I, ¹³¹I.

Means of detecting such detectable markers are well known to those of skill in the art. Thus, for example, radiolabels may be detected using photographic film or scintillation counters, fluorescent markers may be detected using a photodetector to detect emitted illumination. Enzymatic labels are typically detected by providing the enzyme with a substrate and detecting the reaction product produced by the action of the enzyme on the substrate, and colorimetric labels are detected by simply visualizing the colored label.

D. Nucleotides, Expression, Vectors, and Host Cells

Further provided by an embodiment of the invention is a nucleic acid comprising a nucleotide sequence encoding any of the CARs, an antibody, or antigen binding portion thereof, described herein (including functional portions and functional variants thereof). The nucleic acids of the invention may comprise a nucleotide sequence encoding any of the leader sequences, antigen binding domains, transmembrane domains, and/or intracellular T cell signaling domains described herein.

In some embodiments, the nucleotide sequence may be codon-modified. Without being bound to a particular theory, it is believed that codon optimization of the nucleotide sequence increases the translation efficiency of the mRNA transcripts. Codon optimization of the nucleotide sequence may involve substituting a native codon for another codon that encodes the same amino acid, but can be translated by tRNA that is more readily available within a cell, thus increasing translation efficiency. Optimization of the nucleotide sequence may also reduce secondary mRNA structures that would interfere with translation, thus increasing translation efficiency.

In an embodiment of the invention, the nucleic acid may comprise a codon-modified nucleotide sequence that encodes the antigen binding domain of the inventive CAR. In another embodiment of the invention, the nucleic acid may comprise a codon-modified nucleotide sequence that encodes any of the CARs described herein (including functional portions and functional variants thereof).

“Nucleic acid” as used herein includes “polynucleotide,” “oligonucleotide,” and “nucleic acid molecule,” and generally means a polymer of DNA or RNA, which can be single-stranded or double-stranded, synthesized or obtained (e.g., isolated and/or purified) from natural sources, which can contain natural, non-natural or altered nucleotides, and which can contain a natural, non-natural or altered internucleotide linkage, such as a phosphoroamidate linkage or a phosphorothioate linkage, instead of the phosphodiester found between the nucleotides of an unmodified oligonucleotide. In some embodiments, the nucleic acid does not comprise any insertions, deletions, inversions, and/or substitutions. However, it may be suitable in some instances, as discussed herein, for the nucleic acid to comprise one or more insertions, deletions, inversions, and/or substitutions.

A recombinant nucleic acid may be one that has a sequence that is not naturally occurring or has a sequence that is made by an artificial combination of two otherwise separated segments of sequence. This artificial combination is often accomplished by chemical synthesis or, more commonly, by the artificial manipulation of isolated segments of nucleic acids, e.g., by genetic engineering techniques, such as those described in Sambrook et al., supra. The nucleic acids can be constructed based on chemical synthesis and/or enzymatic ligation reactions using procedures known in the art. See, for example, Sambrook el al., supra, and Ausubel et al., supra. For example, a nucleic acid can be chemically synthesized using naturally occurring nucleotides or variously modified nucleotides designed to increase the biological stability of the molecules or to increase the physical stability of the duplex formed upon hybridization (e.g., phosphorothioate derivatives and acridine substituted nucleotides). Examples of modified nucleotides that can be used to generate the nucleic acids include, but are not limited to, 5-fluorouracil, 5-bromouracil, 5-chlorouracil, 5-iodouracil, hypoxanthine, xanthine, 4-acetylcytosine, 5-(carboxyhydroxymethyl) uracil, 5-carboxymethylaminomethyl-2-thiouridine, 5-carboxymethylaminomethyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-substituted adenine, 7-methylguanine, 5-methylaminomethyluracil, 5-methoxyaminomethyl-2-thiouracil, beta-D-mannosylqueosine, 5′-methoxycarboxymethyluracil, 5-methoxyuracil, 2-methylthio-N6-isopentenyladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, 3-(3-amino-3-N-2-carboxypropyl) uracil, and 2,6-diaminopurine. Alternatively, one or more of the nucleic acids of the invention can be purchased from companies, such as Integrated DNA Technologies (Coralville, IA, USA).

The nucleic acid can comprise any isolated or purified nucleotide sequence which encodes any of the CARs or functional portions or functional variants thereof. Alternatively, the nucleotide sequence can comprise a nucleotide sequence which is degenerate to any of the sequences or a combination of degenerate sequences.

An embodiment also provides an isolated or purified nucleic acid comprising a nucleotide sequence which is complementary to the nucleotide sequence of any of the nucleic acids described herein or a nucleotide sequence which hybridizes under stringent conditions to the nucleotide sequence of any of the nucleic acids described herein.

The nucleotide sequence which hybridizes under stringent conditions may hybridize under high stringency conditions. By “high stringency conditions” is meant that the nucleotide sequence specifically hybridizes to a target sequence (the nucleotide sequence of any of the nucleic acids described herein) in an amount that is detectably stronger than non-specific hybridization. High stringency conditions include conditions which would distinguish a polynucleotide with an exact complementary sequence, or one containing only a few scattered mismatches from a random sequence that happened to have a few small regions (e.g., 3-10 bases) that matched the nucleotide sequence. Such small regions of complementarity are more easily melted than a full-length complement of 14-17 or more bases, and high stringency hybridization makes them easily distinguishable. Relatively high stringency conditions would include, for example, low salt and/or high temperature conditions, such as provided by about 0.02-0.1 M NaCl or the equivalent, at temperatures of about 50-70° C. Such high stringency conditions tolerate little, if any, mismatch between the nucleotide sequence and the template or target strand, and are particularly suitable for detecting expression of any of the inventive CARs. It is generally appreciated that conditions can be rendered more stringent by the addition of increasing amounts of formamide.

Also provided is a nucleic acid comprising a nucleotide sequence that is at least about 70% or more, e.g., about 80%, about 90%, about 91%, about 92%, about 93%, about 94%, about 95%, about 96%, about 97%, about 98%, or about 99% identical to any of the nucleic acids described herein.

In an embodiment, the nucleic acids can be incorporated into a recombinant expression vector. In this regard, an embodiment provides recombinant expression vectors comprising any of the nucleic acids. For purposes herein, the term “recombinant expression vector” means a genetically-modified oligonucleotide or polynucleotide construct that permits the expression of an mRNA, protein, polypeptide, or peptide by a host cell, when the construct comprises a nucleotide sequence encoding the mRNA, protein, polypeptide, or peptide, and the vector is contacted with the cell under conditions sufficient to have the mRNA, protein, polypeptide, or peptide expressed within the cell. The vectors are not naturally-occurring as a whole.

However, parts of the vectors can be naturally-occurring. The recombinant expression vectors can comprise any type of nucleotides, including, but not limited to DNA and RNA, which can be single-stranded or double-stranded, synthesized or obtained in part from natural sources, and which can contain natural, non-natural or altered nucleotides. The recombinant expression vectors can comprise naturally-occurring or non-naturally-occurring internucleotide linkages, or both types of linkages. Preferably, the non-naturally occurring or altered nucleotides or internucleotide linkages do not hinder the transcription or replication of the vector.

In an embodiment, the recombinant expression vector can be any suitable recombinant expression vector, and can be used to transform or transfect any suitable host cell. Suitable vectors include those designed for propagation and expansion or for expression or both, such as plasmids and viruses. The vector can be selected from the group consisting of the pUC series (Fermentas Life Sciences, Glen Burnie, MD), the pBluescript series (Stratagene, LaJolla, CA), the pET series (Novagen, Madison, WI), the pGEX series (Pharmacia Biotech, Uppsala, Sweden), and the pEX series (Clontech, Palo Alto, CA).

Bacteriophage vectors, such as λ\hat{ν}TIO, λ\ddot{ν}TI 1, λZapII (Stratagene), EMBL4, and λNMI 149, also can be used. Examples of plant expression vectors include pBIOl, pBI101.2, pBHOl.3, pBI121 and pBIN19 (Clontech). Examples of animal expression vectors include pEUK-Cl, pMAM, and pMAMneo (Clontech). The recombinant expression vector may be a viral vector, e.g., a retroviral vector or a lentiviral vector. A lentiviral vector is a vector derived from at least a portion of a lentivirus genome, including especially a self-inactivating lentiviral vector as provided in Milone et al., Mol. Ther. 17(8): 1453-1464 (2009). Other examples of lentivirus vectors that may be used in the clinic, include, for example, and not by way of limitation, the LENTIVECTOR® gene delivery technology from Oxford BioMedica plc, the LENTIMAX™ vector system from Lentigen and the like. Nonclinical types of lentiviral vectors are also available and would be known to one skilled in the art.

A number of transfection techniques are generally known in the art (see, e.g., Graham et al., Virology, 52: 456-467 (1973): Sambrook et al., supra; Davis et al., Basic Methods in Molecular Biology, Elsevier (1986); and Chu et al, Gene, 13: 97 (1981).

Transfection methods include calcium phosphate co-precipitation (see, e.g., Graham et al., supra), direct micro injection into cultured cells (see, e.g., Capecchi, Cell, 22: 479-488 (1980)), electroporation (see, e.g., Shigekawa et al., BioTechniques, 6: 742-751 (1988)), liposome mediated gene transfer (see, e.g., Mannino et al., BioTechniques, 6: 682-690 (1988)), lipid mediated transduction (see, e.g., Feigner et al., Proc. Natl. Acad. Sci. USA, 84: 7413-7417 (1987)), and nucleic acid delivery using high velocity microprojectiles (see, e.g., Klein et al, Nature, 327: 70-73 (1987)).

In an embodiment, the recombinant expression vectors can be prepared using standard recombinant DNA techniques described in, for example, Sambrook et al., supra, and Ausubel et al., supra. Constructs of expression vectors, which are circular or linear, can be prepared to contain a replication system functional in a prokaryotic or eukaryotic host cell. Replication systems can be derived, e.g., from ColEl, 2μ plasmid, X, SV40, bovine papilloma virus, and the like.

The recombinant expression vector may comprise regulatory sequences, such as transcription and translation initiation and termination codons, which are specific to the type of host cell (e.g., bacterium, fungus, plant, or animal) into which the vector is to be introduced, as appropriate, and taking into consideration whether the vector is DNA- or RNA-based. The recombinant expression vector may comprise restriction sites to facilitate cloning.

The recombinant expression vector can include one or more marker genes, which allow for selection of transformed or transfected host cells. Marker genes include biocide resistance, e.g., resistance to antibiotics, heavy metals, etc., complementation in an auxotrophic host to provide prototrophy, and the like. Suitable marker genes for the inventive expression vectors include, for instance, neomycin/G418 resistance genes, hygromycin resistance genes, histidinol resistance genes, tetracycline resistance genes, and ampicillin resistance genes.

The recombinant expression vector can comprise a native or nonnative promoter operably linked to the nucleotide sequence encoding the CAR (including functional portions and functional variants thereof), or to the nucleotide sequence which is complementary to or which hybridizes to the nucleotide sequence encoding the CAR. The selection of promoters, e.g., strong, weak, inducible, tissue-specific and developmental-specific, is within the ordinary skill of the artisan. Similarly, the combining of a nucleotide sequence with a promoter is also within the skill of the artisan. The promoter can be a non-viral promoter or a viral promoter, e.g., a cytomegalovirus (CMV) promoter, an SV40 promoter, an RSV promoter, or a promoter found in the long-terminal repeat of the murine stem cell virus.

The recombinant expression vectors can be designed for either transient expression, for stable expression, or for both. Also, the recombinant expression vectors can be made for constitutive expression or for inducible expression.

Further, the recombinant expression vectors can be made to include a suicide gene. As used herein, the term “suicide gene” refers to a gene that causes the cell expressing the suicide gene to die. The suicide gene can be a gene that confers sensitivity to an agent, e.g., a drug, upon the cell in which the gene is expressed, and causes the cell to die when the cell is contacted with or exposed to the agent. Suicide genes are known in the art (see, for example, Suicide Gene Therapy: Methods and Reviews, Springer, Caroline J. (Cancer Research UK Centre for Cancer Therapeutics at the Institute of Cancer Research, Sutton, Surrey, UK), Humana Press, 2004) and include, for example, the Herpes Simplex Virus (HSV) thymidine kinase (TK) gene, cytosine daminase, purine nucleoside phosphorylase, and nitroreductase.

An embodiment further provides a host cell comprising any of the recombinant expression vectors described herein. As used herein, the term “host cell” refers to any type of cell that can contain the inventive recombinant expression vector. The host cell can be a eukaryotic cell, e.g., plant, animal, fungi, or algae, or can be a prokaryotic cell, e.g., bacteria or protozoa. The host cell can be a cultured cell or a primary cell, i.e., isolated directly from an organism, e.g., a human. The host cell can be an adherent cell or a suspended cell, i.e., a cell that grows in suspension. Suitable host cells are known in the art and include, for instance, DH5a E. coli cells, Chinese hamster ovarian cells, monkey VERO cells, COS cells, HEK293 cells, and the like. For purposes of amplifying or replicating the recombinant expression vector, the host cell may be a prokaryotic cell, e.g., a DH5a cell. For purposes of producing a recombinant CAR, the host cell may be a mammalian cell. The host cell may be a human cell. While the host cell can be of any cell type, can originate from any type of tissue, and can be of any developmental stage, the host cell may be a peripheral blood lymphocyte (PBL) or a peripheral blood mononuclear cell (PBMC). The host cell may be a T cell.

For purposes herein, the T cell can be any T cell, such as a cultured T cell, e.g., a primary T cell, or a T cell from a cultured T cell line, e.g., Jurkat, SupTl, etc., or a T cell obtained from a mammal. If obtained from a mammal, the T cell can be obtained from numerous sources, including but not limited to blood, bone marrow, lymph node, the thymus, or other tissues or fluids. T cells can also be enriched for or purified. The T cell may be a human T cell. The T cell may be a T cell isolated from a human. The T cell can be any type of T cell and can be of any developmental stage, including but not limited to, CD4+/CD8+ double positive T cells, CD4+ helper T cells, e.g., Th1 and Th2 cells, CD8+ T cells (e.g., cytotoxic T cells), tumor infiltrating cells, memory T cells, memory stem cells, i.e. Tscm, naive T cells, and the like. The T cell may be a CD8+ T cell or a CD4+ T cell.

In an embodiment, the CARs as described herein can be used in suitable non-T cells. Such cells are those with an immune-effector function, such as, for example, NK cells, and T-like cells generated from pluripotent stem cells.

Also provided by an embodiment is a population of cells comprising at least one host cell described herein. The population of cells can be a heterogeneous population comprising the host cell comprising any of the recombinant expression vectors described, in addition to at least one other cell, e.g., a host cell (e.g., a T cell), which does not comprise any of the recombinant expression vectors, or a cell other than a T cell, e.g., a B cell, a macrophage, a neutrophil, an erythrocyte, a hepatocyte, an endothelial cell, an epithelial cell, a muscle cell, a brain cell, etc. Alternatively, the population of cells can be a substantially homogeneous population, in which the population comprises mainly host cells (e.g., consisting essentially of) comprising the recombinant expression vector. The population also can be a clonal population of cells, in which all cells of the population are clones of a single host cell comprising a recombinant expression vector, such that all cells of the population comprise the recombinant expression vector. In one embodiment of the invention, the population of cells is a clonal population comprising host cells comprising a recombinant expression vector as described herein.

CARs (including functional portions and variants thereof), nucleic acids, recombinant expression vectors, host cells (including populations thereof), and antibodies (including antigen binding portions thereof), can be isolated and/or purified. For example, a purified (or isolated) host cell preparation is one in which the host cell is more pure than cells in their natural environment within the body. Such host cells may be produced, for example, by standard purification techniques. In some embodiments, a preparation of a host cell is purified such that the host cell represents at least about 50%, for example at least about 70%, of the total cell content of the preparation. For example, the purity can be at least about 50%, can be greater than about 60%, about 70% or about 80%, or can be about 100%.

E. Methods of Treatment

It is contemplated that the CARs disclosed herein can be used in methods of treating or preventing a disease in a mammal. In this regard, an embodiment provides a method of treating or preventing cancer in a mammal, comprising administering to the mammal the CARs, the nucleic acids, the recombinant expression vectors, the host cells, the population of cells, the antibodies and/or the antigen binding portions thereof, and/or the pharmaceutical compositions in an amount effective to treat or prevent cancer in the mammal.

An embodiment further comprises lymphodepleting the mammal prior to administering the CARs disclosed herein. Examples of lymphodepletion include, but may not be limited to, nonmyeloablative lymphodepleting chemotherapy, myeloablative lymphodepleting chemotherapy, total body irradiation, etc.

For purposes of the methods, wherein host cells or populations of cells are administered, the cells can be cells that are allogeneic or autologous to the mammal. Preferably, the cells are autologous to the mammal. As used herein, allogeneic means any material derived from a different animal of the same species as the individual to whom the material is introduced. Two or more individuals are said to be allogeneic to one another when the genes at one or more loci are not identical. In some aspects, allogeneic material from individuals of the same species may be sufficiently unlike genetically to interact antigenically. As used herein, “autologous” means any material derived from the same individual to whom it is later to be re-introduced into the individual.

The mammal referred to herein can be any mammal. As used herein, the term “mammal” refers to any mammal, including, but not limited to, mammals of the order Rodentia, such as mice and hamsters, and mammals of the order Logomorpha, such as rabbits. The mammals may be from the order Carnivora, including Felines (cats) and Canines (dogs). The mammals may be from the order Artiodactyla, including Bovines (cows) and Swines (pigs) or of the order Perssodactyla, including Equines (horses). The mammals may be of the order Primates, Ceboids, or Simoids (monkeys) or of the order Anthropoids (humans and apes). Preferably, the mammal is a human.

With respect to the methods, the cancer can be any cancer, including any of acute lymphocytic cancer, acute myeloid leukemia, alveolar rhabdomyosarcoma, bladder cancer (e.g., bladder carcinoma), bone cancer, brain cancer (e.g., meduloblastoma), breast cancer, cancer of the anus, anal canal, or anorectum, cancer of the eye, cancer of the intrahepatic bile duct, cancer of the joints, cancer of the neck, gallbladder, or pleura, cancer of the nose, nasal cavity, or middle ear, cancer of the oral cavity, cancer of the vulva, chronic lymphocytic leukemia, chronic myeloid cancer, colon cancer, esophageal cancer, cervical cancer, fibrosarcoma, gastrointestinal carcinoid tumor, head and neck cancer (e.g., head and neck squamous cell carcinoma), Hodgkin lymphoma, hypopharynx cancer, kidney cancer, larynx cancer, leukemia, liquid tumors, liver cancer, lung cancer (e.g., non-small cell lung carcinoma and lung adenocarcinoma), lymphoma, mesothelioma, mastocytoma, melanoma, multiple myeloma, nasopharynx cancer, non-Hodgkin lymphoma, B-chronic lymphocytic leukemia, hairy cell leukemia, acute lymphocytic leukemia (ALL), and Burkitt's lymphoma, ovarian cancer, pancreatic cancer, peritoneum, omentum, and mesentery cancer, pharynx cancer, prostate cancer, rectal cancer, renal cancer, skin cancer, small intestine cancer, soft tissue cancer, solid tumors, synovial sarcoma, gastric cancer, testicular cancer, thyroid cancer, and ureter cancer.

The terms “treat,” and “prevent” as well as words stemming therefrom, as used herein, do not necessarily imply 100% or complete treatment or prevention. Rather, there are varying degrees of treatment or prevention of which one of ordinary skill in the art recognizes as having a potential benefit or therapeutic effect. In this respect, the methods can provide any amount or any level of treatment or prevention of cancer in a mammal.

Furthermore, the treatment or prevention provided by the method can include treatment or prevention of one or more conditions or symptoms of the disease, e.g., cancer, being treated or prevented. Also, for purposes herein, “prevention” can encompass delaying the onset of the disease, or a symptom or condition thereof.

Another embodiment provides a method of detecting the presence of cancer in a mammal, comprising: (a) contacting a sample comprising one or more cells from the mammal with the CARs, the nucleic acids, the recombinant expression vectors, the host cells, the population of cells, the antibodies, and/or the antigen binding portions thereof, or the pharmaceutical compositions, thereby forming a complex, (b) and detecting the complex, wherein detection of the complex is indicative of the presence of cancer in the mammal.

The sample may be obtained by any suitable method, e.g., biopsy or necropsy. A biopsy is the removal of tissue and/or cells from an individual. Such removal may be to collect tissue and/or cells from the individual in order to perform experimentation on the removed tissue and/or cells. This experimentation may include experiments to determine if the individual has and/or is suffering from a certain condition or disease-state. The condition or disease may be, e.g., cancer.

With respect to an embodiment of the method of detecting the presence of a proliferative disorder, e.g., cancer, in a mammal, the sample comprising cells of the mammal can be a sample comprising whole cells, lysates thereof, or a fraction of the whole cell lysates, e.g., a nuclear or cytoplasmic fraction, a whole protein fraction, or a nucleic acid fraction. If the sample comprises whole cells, the cells can be any cells of the mammal, e.g., the cells of any organ or tissue, including blood cells or endothelial cells.

The contacting can take place in vitro or in vivo with respect to the mammal. Preferably, the contacting is in vitro.

Also, detection of the complex can occur through any number of ways known in the art. For instance, the CARs disclosed herein, polypeptides, proteins, nucleic acids, recombinant expression vectors, host cells, populations of cells, or antibodies, or antigen binding portions thereof, described herein, can be labeled with a detectable label such as, for instance, a radioisotope, a fluorophore (e.g., fluorescein isothiocyanate (FITC), phycoerythrin (PE)), an enzyme (e.g., alkaline phosphatase, horseradish peroxidase), and element particles (e.g., gold particles) as disclosed supra.

Methods of testing a CAR for the ability to recognize target cells and for antigen specificity are known in the art. For instance, Clay et al., J. Immunol, 163: 507-513 (1999), teaches methods of measuring the release of cytokines (e.g., interferon-y, granulocyte/monocyte colony stimulating factor (GM-CSF), tumor necrosis factor a (TNF-a) or interleukin 2 (IL-2)). In addition, CAR function can be evaluated by measurement of cellular cytotoxicity, as described in Zhao et al, J. Immunol, 174: 4415-4423 (2005).

Another embodiment provides for the use of the CARs, nucleic acids, recombinant expression vectors, host cells, populations of cells, antibodies, or antigen binding portions thereof, and/or pharmaceutical compositions of the invention, for the treatment or prevention of a proliferative disorder, e.g., cancer, in a mammal. The cancer may be any of the cancers described herein.

Any method of administration can be used for the disclosed therapeutic agents, including local and systemic administration. For example topical, oral, intravascular such as intravenous, intramuscular, intraperitoneal, intranasal, intradermal, intrathecal and subcutaneous administration can be used. The particular mode of administration and the dosage regimen will be selected by the attending clinician, taking into account the particulars of the case (for example the subject, the disease, the disease state involved, and whether the treatment is prophylactic). In cases in which more than one agent or composition is being administered, one or more routes of administration may be used; for example, a chemotherapeutic agent may be administered orally and an antibody or antigen binding fragment or conjugate or composition may be administered intravenously. Methods of administration include injection for which the CAR, CAR T Cell, conjugates, antibodies, antigen binding fragments, or compositions are provided in a nontoxic pharmaceutically acceptable carrier such as water, saline, Ringer's solution, dextrose solution, 5% human serum albumin, fixed oils, ethyl oleate, or liposomes. In some embodiments, local administration of the disclosed compounds can be used, for instance by applying the antibody or antigen binding fragment to a region of tissue from which a tumor has been removed, or a region suspected of being prone to tumor development. In some embodiments, sustained intra-tumoral (or near-tumoral) release of the pharmaceutical preparation that includes a therapeutically effective amount of the antibody or antigen binding fragment may be beneficial. In other examples, the conjugate is applied as an eye drop topically to the cornea, or intravitreally into the eye.

The disclosed therapeutic agents can be formulated in unit dosage form suitable for individual administration of precise dosages. In addition, the disclosed therapeutic agents may be administered in a single dose or in a multiple dose schedule. A multiple dose schedule is one in which a primary course of treatment may be with more than one separate dose, for instance 1-10 doses, followed by other doses given at subsequent time intervals as needed to maintain or reinforce the action of the compositions. Treatment can involve daily or multi-daily doses of compound(s) over a period of a few days to months, or even years. Thus, the dosage regime will also, at least in part, be determined based on the particular needs of the subject to be treated and will be dependent upon the judgment of the administering practitioner.

Typical dosages of the antibodies or conjugates can range from about 0.01 to about 30 mg/kg, such as from about 0.1 to about 10 mg/kg.

In particular examples, the subject is administered a therapeutic composition that includes one or more of the conjugates, antibodies, compositions, CARs, CAR T cells or additional agents, on a multiple daily dosing schedule, such as at least two consecutive days, 10 consecutive days, and so forth, for example for a period of weeks, months, or years. In one example, the subject is administered the conjugates, antibodies, compositions or additional agents for a period of at least 30 days, such as at least 2 months, at least 4 months, at least 6 months, at least 12 months, at least 24 months, or at least 36 months.

In some embodiments, the disclosed methods include providing surgery, radiation therapy, and/or chemotherapeutics to the subject in combination with a disclosed antibody, antigen binding fragment, conjugate, CAR or T cell expressing a CAR (for example, sequentially, substantially simultaneously, or simultaneously). Methods and therapeutic dosages of such agents and treatments are known to those skilled in the art, and can be determined by a skilled clinician. Preparation and dosing schedules for the additional agent may be used according to manufacturer's instructions or as determined empirically by the skilled practitioner. Preparation and dosing schedules for such chemotherapy are also described in Chemotherapy Service, (1992) Ed., M. C. Perry, Williams & Wilkins, Baltimore, Md.

In some embodiments, the combination therapy can include administration of a therapeutically effective amount of an additional cancer inhibitor to a subject. Non-limiting examples of additional therapeutic agents that can be used with the combination therapy include microtubule binding agents, DNA intercalators or cross-linkers, DNA synthesis inhibitors, DNA and RNA transcription inhibitors, antibodies, enzymes, enzyme inhibitors, gene regulators, and angiogenesis inhibitors. These agents (which are administered at a therapeutically effective amount) and treatments can be used alone or in combination. For example, any suitable anti-cancer or anti-angiogenic agent can be administered in combination with the CARS, CAR-T cells, antibodies, antigen binding fragment, or conjugates disclosed herein. Methods and therapeutic dosages of such agents are known to those skilled in the art, and can be determined by a skilled clinician.

Additional chemotherapeutic agents include, but are not limited to alkylating agents, such as nitrogen mustards (for example, chlorambucil, chlormethine, cyclophosphamide, ifosfamide, and melphalan), nitrosoureas (for example, carmustine, fotemustine, lomustine, and streptozocin), platinum compounds (for example, carboplatin, cisplatin, oxaliplatin, and BBR3464), busulfan, dacarbazine, mechlorethamine, procarbazine, temozolomide, thiotepa, and uramustine; antimetabolites, such as folic acid (for example, methotrexate, pemetrexed, and raltitrexed), purine (for example, cladribine, clofarabine, fludarabine, mercaptopurine, and tioguanine), pyrimidine (for example, capecitabine), cytarabine, fluorouracil, and gemcitabine; plant alkaloids, such as podophyllum (for example, etoposide, and teniposide), taxane (for example, docetaxel and paclitaxel), vinca (for example, vinblastine, vincristine, vindesine, and vinorelbine); cytotoxic/antitumor antibiotics, such as anthracycline family members (for example, daunorubicin, doxorubicin, epirubicin, idarubicin, mitoxantrone, and valrubicin), bleomycin, rifampicin, hydroxyurea, and mitomycin; topoisomerase inhibitors, such as topotecan and irinotecan; monoclonal antibodies, such as alemtuzumab, bevacizumab, cetuximab, gemtuzumab, rituximab, panitumumab, pertuzumab, and trastuzumab; photosensitizers, such as aminolevulinic acid, methyl aminolevulinate, porfimer sodium, and verteporfin; and other agents, such as alitretinoin, altretamine, amsacrine, anagrelide, arsenic trioxide, asparaginase, axitinib, bexarotene, bevacizumab, bortezomib, celecoxib, denileukin diftitox, erlotinib, estramustine, gefitinib, hydroxycarbamide, imatinib, lapatinib, pazopanib, pentostatin, masoprocol, mitotane, pegaspargase, tamoxifen, sorafenib, sunitinib, vemurafinib, vandetanib, and tretinoin. Selection and therapeutic dosages of such agents are known to those skilled in the art, and can be determined by a skilled clinician.

The combination therapy may provide synergy and prove synergistic, that is, the effect achieved when the active ingredients used together is greater than the sum of the effects that results from using the compounds separately. A synergistic effect may be attained when the active ingredients are: (1) co-formulated and administered or delivered simultaneously in a combined, unit dosage formulation; (2) delivered by alternation or in parallel as separate formulations; or (3) by some other regimen. When delivered in alternation, a synergistic effect may be attained when the compounds are administered or delivered sequentially, for example by different injections in separate syringes. In general, during alternation, an effective dosage of each active ingredient is administered sequentially, i.e. serially, whereas in combination therapy, effective dosages of two or more active ingredients are administered together.

In one embodiment, an effective amount of an antibody or antigen binding fragment that specifically binds to one or more of the antigens disclosed herein or a conjugate thereof is administered to a subject having a tumor following anti-cancer treatment. After a sufficient amount of time has elapsed to allow for the administered antibody or antigen binding fragment or conjugate to form an immune complex with the antigen expressed on the respective cancer cell, the immune complex is detected. The presence (or absence) of the immune complex indicates the effectiveness of the treatment. For example, an increase in the immune complex compared to a control taken prior to the treatment indicates that the treatment is not effective, whereas a decrease in the immune complex compared to a control taken prior to the treatment indicates that the treatment is effective.

F. Biopharmaceutical Compositions

Biopharmaceutical or biologics compositions (hereinafter, “compositions”) are provided herein for use in gene therapy, immunotherapy and/or cell therapy that include one or more of the disclosed CARs, or T cells expressing a CAR, antibodies, antigen binding fragments, conjugates, CARs, or T cells expressing a CAR that specifically bind to one or more antigens disclosed herein, in a carrier (such as a pharmaceutically acceptable carrier). The compositions can be prepared in unit dosage forms for administration to a subject. The amount and timing of administration are at the discretion of the treating clinician to achieve the desired outcome. The compositions can be formulated for systemic (such as intravenus) or local (such as intra-tumor) administration. In one example, a disclosed CARs, or T cells expressing a CAR, antibody, antigen binding fragment, conjugate, is formulated for parenteral administration, such as intravenous administration. Compositions including a CAR, or T cell expressing a CAR, a conjugate, antibody or antigen binding fragment as disclosed herein are of use, for example, for the treatment and detection of a tumor, for example, and not by way of limitation, a neuroblastoma. In some examples, the compositions are useful for the treatment or detection of a carcinoma. The compositions including a CAR, or T cell expressing a CAR, a conjugate, antibody or antigen binding fragment as disclosed herein are also of use, for example, for the detection of pathological angiogenesis.

The compositions for administration can include a solution of the CAR, or T cell expressing a CAR, conjugate, antibody or antigen binding fragment dissolved in a pharmaceutically acceptable carrier, such as an aqueous carrier. A variety of aqueous carriers can be used, for example, buffered saline and the like. These solutions are sterile and generally free of undesirable matter. These compositions may be sterilized by conventional, well known sterilization techniques. The compositions may contain pharmaceutically acceptable auxiliary substances as required to approximate physiological conditions such as pH adjusting and buffering agents, toxicity adjusting agents, adjuvant agents, and the like, for example, sodium acetate, sodium chloride, potassium chloride, calcium chloride, sodium lactate and the like. The concentration of a CAR, or T cell expressing a CAR, antibody or antigen binding fragment or conjugate in these formulations can vary widely, and will be selected primarily based on fluid volumes, viscosities, body weight and the like in accordance with the particular mode of administration selected and the subject's needs. Actual methods of preparing such dosage forms for use in in gene therapy, immunotherapy and/or cell therapy are known, or will be apparent, to those skilled in the art.

A typical composition for intravenous administration includes about 0.01 to about 30 mg/kg of antibody or antigen binding fragment or conjugate per subject per day (or the corresponding dose of a CAR, or T cell expressing a CAR, conjugate including the antibody or antigen binding fragment). Actual methods for preparing administrable compositions will be known or apparent to those skilled in the art and are described in more detail in such publications as Remington's Pharmaceutical Science, 19th ed., Mack Publishing Company, Easton, PA (1995).

A CAR, or T cell expressing a CAR, antibodies, antigen binding fragments, or conjugates may be provided in lyophilized form and rehydrated with sterile water before administration, although they are also provided in sterile solutions of known concentration. The CARs, or T cells expressing a CAR, antibody or antigen binding fragment or conjugate solution is then added to an infusion bag containing 0.9% sodium chloride, USP, and in some cases administered at a dosage of from 0.5 to 15 mg/kg of body weight. Considerable experience is available in the art in the administration of antibody or antigen binding fragment and conjugate drugs; for example, antibody drugs have been marketed in the U.S. since the approval of RITUXAN® in 1997. A CAR, or T cell expressing a CAR, antibodies, antigen binding fragments and conjugates thereof can be administered by slow infusion, rather than in an intravenous push or bolus. In one example, a higher loading dose is administered, with subsequent, maintenance doses being administered at a lower level. For example, an initial loading dose of 4 mg/kg antibody or antigen binding fragment (or the corresponding dose of a conjugate including the antibody or antigen binding fragment) may be infused over a period of some 90 minutes, followed by weekly maintenance doses for 4-8 weeks of 2 mg/kg infused over a 30 minute period if the previous dose was well tolerated.

Controlled release parenteral formulations can be made as implants, oily injections, or as particulate systems. For a broad overview of protein delivery systems see, Banga, A. J., Therapeutic Peptides and Proteins: Formulation, Processing, and Delivery Systems, Technomic Publishing Company, Inc., Lancaster, PA, (1995). Particulate systems include microspheres, microparticles, microcapsules, nanocapsules, nanospheres, and nanoparticles. Microcapsules contain the therapeutic protein, such as a cytotoxin or a drug, as a central core. In microspheres, the therapeutic is dispersed throughout the particle. Particles, microspheres, and microcapsules smaller than about 1 μm are generally referred to as nanoparticles, nanospheres, and nanocapsules, respectively. Capillaries have a diameter of approximately 5 μm so that only nanoparticles are administered intravenously. Microparticles are typically around 100 μm in diameter and are administered subcutaneously or intramuscularly. See, for example, Kreuter, J., Colloidal Drug Delivery Systems, J. Kreuter, ed., Marcel Dekker, Inc., New York, NY, pp. 219-342 (1994); and Tice & Tabibi, Treatise on Controlled Drug Delivery, A. Kydonieus, ed., Marcel Dekker, Inc. New York, NY, pp. 315-339, (1992).

Polymers can be used for ion-controlled release of the CARs, or T cells expressing a CAR, antibody or antigen binding fragment or conjugate compositions disclosed herein. Various degradable and nondegradable polymeric matrices for use in controlled drug delivery are known in the art (Langer, Accounts Chem. Res. 26:537-542, 1993). For example, the block copolymer, polaxamer 407, exists as a viscous yet mobile liquid at low temperatures but forms a semisolid gel at body temperature. It has been shown to be an effective vehicle for formulation and sustained delivery of recombinant interleukin-2 and urease (Johnston et al., Pharm. Res. 9:425-434, 1992; and Pec et al., J. Parent. Sci. Tech. 44(2):58-65, 1990). Alternatively, hydroxyapatite has been used as a microcarrier for controlled release of proteins (Ijntema et al., Int. J. Pharm. 112:215-224, 1994). In yet another aspect, liposomes are used for controlled release as well as drug targeting of the lipid-capsulated drug (Betageri et al., Liposome Drug Delivery Systems, Technomic Publishing Co., Inc., Lancaster, PA (1993)). Numerous additional systems for controlled delivery of therapeutic proteins are known (see U.S. Pat. Nos. 5,055,303; 5,188,837; 4,235,871; 4,501,728; 4,837,028; 4,957,735; 5,019,369; 5,055,303; 5,514,670; 5,413,797; 5,268,164; 5,004,697; 4,902,505; 5,506,206; 5,271,961; 5,254,342 and 5,534,496).

G. Kits

In one aspect, kits employing the CARs disclosed herein are also provided. For example, kits for treating a tumor in a subject, or making a CAR T cell that expresses one or more of the CARs disclosed herein. The kits will typically include a disclosed antibody, antigen binding fragment, conjugate, nucleic acid molecule, CAR or T cell expressing a CAR as disclosed herein. More than one of the disclosed antibodies, antigen binding fragments, conjugates, nucleic acid molecules, CARs or T cells expressing a CAR can be included in the kit.

The kit can include a container and a label or package insert on or associated with the container. Suitable containers include, for example, bottles, vials, syringes, etc. The containers may be formed from a variety of materials such as glass or plastic. The container typically holds a composition including one or more of the disclosed antibodies, antigen binding fragments, conjugates, nucleic acid molecules, CARs or T cells expressing a CAR. In several embodiments the container may have a sterile access port (for example the container may be an intravenous solution bag or a vial having a stopper pierceable by a hypodermic injection needle). A label or package insert indicates that the composition is used for treating the particular condition.

The label or package insert typically will further include instructions for use of a disclosed antibodies, antigen binding fragments, conjugates, nucleic acid molecules, CARs or T cells expressing a CAR, for example, in a method of treating or preventing a tumor or of making a CAR T cell. The package insert typically includes instructions customarily included in commercial packages of therapeutic products that contain information about the indications, usage, dosage, administration, contraindications and/or warnings concerning the use of such therapeutic products. The instructional materials may be written, in an electronic form (such as a computer diskette or compact disk) or may be visual (such as video files). The kits may also include additional components to facilitate the particular application for which the kit is designed. Thus, for example, the kit may additionally contain means of detecting a label (such as enzyme substrates for enzymatic labels, filter sets to detect fluorescent labels, appropriate secondary labels such as a secondary antibody, or the like). The kits may additionally include buffers and other reagents routinely used for the practice of a particular method. Such kits and appropriate contents are well known to those of skill in the art.

### EXAMPLES

This invention is further illustrated by the following examples, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

### Example 1

Development of CD19 and CD20 Dual Targeting CAR T Constructs

Adoptive immunotherapy for cancer with genetically engineered autologous human T cells is currently being evaluated in numerous centers. One common approach to creating a cell population for adoptive immunotherapy is to isolate T cells by apheresis from the patient and to transduce these cells ex vivo with retroviral or lentiviral vectors that integrate into the host genome and express a chimeric antigen receptor (CAR), reviewed in [1]. Chimeric antigen receptors are created by linking functional sequence domains from different subunits of immunologically active proteins. For example, an scFv domain created from the VH and VL domains of an anti-CD19 or anti-CD20 antibody can be linked to transmembrane sequences derived from CD28 or CD8, and then linked to the intracellular signaling domains derived from the CD3-zeta chain and CD28 or CD137 [2, 3]. The CAR thus confers both a binding domain derived from the scFv and the linked signaling domains in a single transmembrane protein that allows activation of a vector-transduced T cells. This transduced T cell population (CAR-T) can now functionally target cells bearing the cognate antigen for destruction by active cytolysis, and by indirect immune effector mechanisms marshaled by the production of cytokines, such as interferon-gamma (IFNγ), interleukin-2 (IL-2), and tumor necrosis factor-alpha (TNFα). Adoptive immunotherapy with chimeric antigen receptor modified T cells that specifically target CD19 has proven efficacy against pediatric pre-B ALL [4, 5]. The effectiveness of CAR-modified T cells in adult hematologic malignancies has been more heterogeneous.

While the experience with anti-CD19 CAR-T therapy at the University of Pennsylvania with 3 CLL patients seemed to indicate a universal positive response, the Surgery Branch at the NCI reported a mixture of partial responses, stable disease, and one complete response in a diverse collection of 8 patients with adult B cell malignancies [6, 7]. Thus, the anti-CD19 CAR is not universally effective and may benefit from further enhancement of its anti-tumor targeting potential. The laboratory of Thomas-Tikhonenko has elegantly described the escape mechanisms employed by B-ALL during anti-CD19 CAR-T therapy which include alternative splicing of CD19, frameshift mutations, and missense mutations [8]. One means to both broaden the target range of a CAR-T product as well as to target malignancies with greater effect is to include two binding domains in a single CAR structure. Tandem CD19- and CD20-expressing malignancies include chronic lymphocytic leukemia (CLL), hairy cell leukemia (HCL), mantle cell lymphoma (MCL), prolymphocytic leukemia (PLL), and splenic lymphoma with villous lymphocytes (SLVL) [9]. A single CAR vector targeting both antigens would target a broader variety of hematologic malignancies, and potentially target them more effectively.

In this Example, we describe the development of fully human CAR t cells targeting Cd19 and CD20 tumor antigens simultaneously to prevent tumor antigen escape. CAR T optimal architecture and selection of the most effective hinge and transmembrane CAR domains is also described.

Materials and Methods

Cell Lines

The Burkitt lymphoma cell line Raji was purchased from American Tissue Culture Collection (ATCC, Manassas, VA). The REH and the NALM-6 leukemia lines were purchased from DSMZ (Leibniz Institute DSMZ, Braunschweig, Germany). Cells were cultured in RPMI-1640 medium (Corning, NY) supplemented with 10% heat-inactivated fetal bovine serum (FBS, Hyclone, Logan, UT). Single-cell clones of luciferase and GFP expressing cell lines were generated by stably transducing wild-type tumor lines with lentiviral vector encoding firefly luciferase and GFP connected with 2A peptide (Lentigen Technology, Inc., Gaithersburg, MD), followed by selection of luciferase-positive clones. Human Embryonic kidney line 293T was purchased from ATCC and cultured in Dynamis medium (Gibco/Thermo Fisher Scientific, Grand Island, NY) supplemented with 4 mM L-Glutamine (Lonza, Morristown, NJ). Luciferase-expressing 293T cell lines were generated by stably transducing wild-type tumor lines with lentiviral vector encoding firefly luciferase (Lentigen Technology, Inc., Gaithersburg, MD), followed by selection of luciferase-positive cells.

Generation of CAR Constructs and Lentiviral Vector Production

The human anti-CD19 and CD20 chimeric antigen receptor (CAR) constructs were designed as tandem CAR or biscistronic CAR. The various single chain variable fragment (ScFv) sequence targeting the extracellular domain of human CD19 was identified in house, the ScFv targeting CD20 was derived from Ofatumumab. For the tandem CARs, antiCD20 scFv connected with antiCD19 scFv via G4S linker, followed by CD8 or CD28 hinge transmembrane, 4-1BB or CD28_4-1BB costimulatory domain(s), and CD3-ζ activating domain sequences. The bicistronic CARs comprised of a CD20-targeting mono CAR, and a mono CD19-targeting CAR, connected with P2A ribosomal skipping element. Mono-CAR constructs used CD8 hinge region, CD8 or OX40 transmembrane, co-stimulatory domains derived from human 4-1BB, CD28, ICOS, OX40 proteins, and CD3-ζ activating domain. Previous developed tandem CAR20_19 with mouse CD19 and CD20 scFvs (LTG1497) was included as control construct. (D. Schneider et al, A tandem CD19/CD20 CAR lentiviral vector drives on-target and off-target antigen modulation in leukemia cell lines. Journal for immunotherapy of cancer 5, 42, 2017).CAR sequences were cloned into a Lentiviral Vector (LV) expression cassette under the control of the human EF-1α promoter (Lentigen Technology Inc., Gaithersburg, MD). Lentiviral particles were generated by transient transfection of HEK 293T cells, pelleted by centrifugation and stored at −80° C. until transduction.

Primary T Cell Preparation and Transduction

Healthy donor primary T cells were isolated either from leukapheresis collections (AllCells, Alameda, CA) or from processed buffy coats (Oklahoma Blood Institute, Tulsa, OK), obtained with donors' written consent. The CD4-positive and CD8-positive human T cells were purified via positive selection using a 1:1 mixture of CD4 and CD8 Microbeads (Miltenyi Biotec, Bergisch Gladbach, Germany) according to manufacturer's protocol. Purified T cells were activated with CD3/CD28 MACS® GMP T Cell TransAct reagent (Miltenyi Biotec), and cultured in serum free TexMACS medium supplemented with 30 IU/ml IL-2 at a density of 1×10⁶ cells/ml. Further, activated T cells were transduced at MOI 80 on day 1 with lentiviral vector particles encoding CAR constructs. On day 3, the transduced T cells were washed and resuspended to 0.5×10⁶/ml to continue expansion. Every 2-3 days thereafter, cultures were supplemented with fresh TexMACS medium containing 30 IU/ml I1-2, until harvest on day 8-10.

Flow Cytometric Analysis of CAR Surface Expression

Half million CAR T cells were washed in cold AutoMACS buffer supplemented with 0.5% bovine serum albumin (Miltenyi Biotec, Bergisch Gladbach, Germany) and stained with CD19-Fc peptide (R&D System, Minneapolis, MN), followed by anti Fc-AF647 conjugate (Jackson ImmunoResearch, West Grove, PA). The 7-Aminoactinomycin D staining (7-AAD, BD Biosciences, San Jose, CA) was added to exclude dead cells. CD4 antibody labeled with Vioblue fluorochrome was used to separate CD4 and CD8 population. Non-transduced cells (UTD) were used as a negative control. Cells were washed twice, resuspended in 200 μl running buffer, and acquired by flow cytometry. Flow cytometric analysis was performed on a MACSQuant® 10 Analyzer (Miltenyi Biotec), and data plots were generated using FlowJo software (Ashland, OR).

CAR T Cell Cytotoxicity and Cytokine Assay

To assess CAR T cell mediated cytotoxicity, 5×10³ tumor target cells stably transduced with firefly luciferase were combined with CAR T cells at the indicated effector to target ratios and incubated overnight at 37° C. with 5% CO₂. SteadyGlo reagent (Promega, Madison WI) was added to each well and the resulting luminescence quantified as counts per second (sample CPS). Target only wells (max CPS) and target only wells plus 1% Tween-20 (min CPS) were used to determine assay range. Percent specific lysis was calculated as: (1-(sample CPS-min CPS)/(max CPS-min CPS)). For cytokine release analysis, 5×10⁴ effectors and 5×10³ targets were co-cultured overnight, and supernatants from co-cultures were removed and analyzed by ELISA (eBioscience, San Diego, CA) for IFNγ, TNFα and IL-2 concentration. Three technical replicates were performed for each condition, and each experiment was repeated using CAR T cells generated from different healthy donors.

Long-Term CAR T and Tumor Cell Co-Incubation Assay

CD19 and CD20 dual targeting CAR T cells or non-transduced control T cells(1×10⁵) were co-cultured with 1×10⁶ Raji lymphoma cells at effector to target ratio of 0.3:1 for 7 days, supplanted with fresh medium when needed. At day 4 and day7 point, cell mixtures were analyzed by flow cytometry. CountBright™ absolute counting beads (Thermofisher, Eugene, OR) were included into cell mixture to obtain absolute cell counts. After exclusion of dead cells by 7-AAD staining. The live T (CD3⁺) counts and percentage of tumor cells (GFP⁺) for each condition was recorded. At day 7, when the Raji cell % is less than 10% in most of cell mixtures, fresh Raji cells were added to coculture mix to re-adjust E:T ratio to 0.15:1. At day 10, culture products were re-evaluated by flow to assess each CAR T cell cytotoxicity.

Results

Example 1 describes the generation and in vitro evaluation of CAR T cells targeting both CD20 and CD19 antigen for the treatment of B cell malignancy.

Schematic representations of the bispecific CAR constructs targeting the both CD19 and CD20 antigen are shown in FIG. 1A. Fully human binder of CAR19 was developed in house, and the fully human scFv targeting CD20 was derived from Ofatumumab. Tandem CARs designed as a CD20 scFv connected with a CD19 scFv through G4S linker, then in frame to CD8 or CD28 hinge and transmembrane, 4-1BB or CD28_4-1BB co-stimulatory domain and CD3ζ activation domain. Duo CAR constructs configured with a mono CD20 CAR and a mono CD19 CAR separated by P2A sequence. Mono CARs comprised of CD19 or CD20 scFv, CD8 hinge, transmembrane domain from CD8 or OX40, co-stimulatory domains from 4-1BB, CD28, OX40 or ICOS, and a CD3ζ activation domain. CAR sequences were further incorporated into a third-generation lentiviral vectors and transduced into human primary T cells at MOI 80, to generate the CD20_19 CAR T cells under the control of the mammalian EF-1α promoter. Previously evaluated CAR construct LTG1497 with mouse binders was also included as positive control while un-transduced T cells derived from same donor (UTD) were used as negative control. CAR19 surface expression of transduced T cells was measured by flow cytometry using CD19-Fc, followed by staining with anti-Fc Alexa Flour 647. CAR20 expression was not been pursued due to lack of detection reagent. Different CD20_19 CAR constructs exhibited surface higher expression (n=4 donors) than positive control LTG1497. Flow plots (CD4 vs CAR19) from one representative donor were shown in FIG. 1B. For this donor, LTG1497 showed lowest surface expression (49.7%), followed by D0144 (58.0%), and other CAR20_19 constructs were ranged 60% to 80%, demonstrating robust transduction efficiency and CAR expression.

To evaluate the target specific cytotoxicity of CD20_19 CARs in vitro, CD19⁺CD20⁺ leukemia or lymphoma lines (Raji, Nalm-6 and Reh) and CD19⁻CD20⁻ non-leukemic lines 293T were selected as target lines. CAR-T cells were co-incubated with target tumor cell lines at effector to target ratios 2.5:1; 5:1 and 10:1. After overnight co-incubation, cytotoxicity of CARs was analyzed in a luminescence based in vitro killing assays (FIG. 2A-C). In CD19⁺ cell lines, Raji, Nalm-6 and Reh, CAR20_19 constructs showed similar or higher killing capacity, compared to CAR LTG1497, at all E:T ratios tested. No cytotoxicity was observed in UTD group in Raji and Reh lines, and some killing was observed for the higher E:T ratios of UTD cells co-incubated with NALM-6 (background killing). Furthermore, no killing or limited background killing of CD19 and CD20 negative 293T cell line (FIG. 2D) was observed, demonstrating the robust target-specific cytotoxic function of all CD20_19 CAR constructs designed.

Production of the T cell homeostatic and pro-inflammatory cytokines IL-2, IFNγ, and TNFα by the fully human CD20_19 CARs and control construct LTG1497, was examined by ELISA. Culture supernatants after overnight co-incubation of CAR T cells with Raji target line at an E:T ratio of 10 (FIG. 4A-4C) was harvested for the measurement of specific cytokine release. Fully human CAR20_19 constructs exhibited significant cytokine response when compared to UTD or CAR alone controls, indicating robust, target-specific CAR T cell cytokine responses. The intensity of cytokine release of each CAR20_19 construct was comparable or higher than control construct LTG1497.

Long term co-culture assay was designed to further characterize CAR20_19 constructs. CAR T cells were co-incubated with Raji cells at low E:T ratio 0.3:1, UTD cells were included as negative control. The culture mix of CAT T and Raji cells was measured with flow cytometry to exam the percentage of remaining Raji cells and T cell fold expansion during the co-culture (FIG. 4 B, 4C). At day 4, in the coculture products of Raji and CAR T cells, Raji cell percentage was reduced and CAR T cell expansion was observed. In contrast, in the coculture of Raji with UTD cells, percentage of Raji cells was increased and T cells did not show proliferation. Furthermore, the CAR D0256 and D0266 showed highest Raji clearance. At day7, all Raji cells were killed by T cells except UTD group. CD20_19 CARs, showed close fold expansion at the end of first round co-culture in comparison with control CAR LTG1497. Additional Raji cells were added to the coculture to re-adjusting ET ration as 0.15:1. At day 10, the killing potency among the group could be distinguished. Notably, CAR D0266 exhibited best killing capability during second round co-culture, followed by D0255 and D0257>D0258>D0256 and control LTG1497. D0144 CAR only had very few killing ability (FIG. 4B). CAR T cells continued to proliferate during the 2^{nd} round coincubation. CAR LTG1497, D0256 and D0144 exhibited slightly more proliferation than other CAR 20_19s. Similar trend was observed in a different donor most CAR 20_19s maintained higher killing potency than control CAR1497, except CAR D0144 (data not shown).

Among all CAR20_19 constructs, D0144 showed the lowest transduction efficiency, and less cytotoxic function during long term coculture. Compared to LTG1497, all other CAR20_19 construct demonstrated high CAR transduction efficiency, similar or stronger cytotoxicity in overnight or long tern killing assay, specific cytokine induction in vitro. In conclusion, all CAR20_19 constructs except D0144 were proceed to in vivo tumor clearance potency evaluation.

### References for Example 1

1. Lee, D. W., et al., The future is now: chimeric antigen receptors as new targeted therapies for childhood cancer. Clinical Cancer Research, 2012. 18(10): p. 2780-2790.
2. Kochenderfer, J. N., et al., Construction and pre-clinical evaluation of an anti-CD19 chimeric antigen receptor. Journal of immunotherapy (Hagerstown, Md.: 1997), 2009. 32(7): p. 689.
3. Jensen, M., et al., CD20 is a molecular target for scFvFc: zeta receptor redirected T cells: implications for cellular immunotherapy of CD20+ malignancy. Biology of Blood and Marrow Transplantation, 1998. 4(2). p. 75-83.
4. Lee, D. W., et al., T cells expressing CD19 chimeric antigen receptors for acute lymphoblastic leukaemia in children and young adults: a phase 1 dose-escalation trial. The Lancet, 2015. 385(9967): p. 517-528.
5. Grupp, S. A., et al., Chimeric antigen receptor-modified T cells for acute lymphoid leukemia. New England Journal of Medicine, 2013. 368(16): p. 1509-1518.
6. Porter, D. L., et al., Chimeric antigen receptor-modified T cells in chronic lymphoid leukemia. New England Journal of Medicine, 2011. 365(8): p. 725-733.
7. Kochenderfer, J. N., et al., B-cell depletion and remissions of malignancy along with cytokine-associated toxicity in a clinical trial of anti-CD19 chimeric-antigen-receptor-transduced T cells. Blood, 2012. 119(12): p. 2709-2720.
8. Sotillo, E., et al., Convergence of acquired mutations and alternative splicing of CD19 enables resistance to CART-19 immunotherapy. Cancer discovery, 2015. 5(12): p. 1282-1295.
9. Ginaldi, L., et al., Levels of expression of CD19 and CD20 in chronic B cell leukaemias.

Journal of clinical pathology, 1998. 51(5): p. 364-369.

### Sequences of the Disclosure

The nucleic and amino acid sequences listed below are shown using standard letter abbreviations for nucleotide bases, and three letter code for amino acids, as defined in 37 C.F.R. 1.822. Only one strand of each nucleic acid sequence is shown, but the complementary strand is understood as included by any reference to the displayed strand. In the accompanying sequence listing:

nucleotide sequence of CD20-reactive
ScFv1 binding domain
SEQ ID NO: 1
GAAATTGTGTTGACTCAGTCACCGGCAACTCTTAGTCTCTCCCCT
GGAGAGAGAGCAACACTGAGCTGCAGAGCATCTCAAAGTGTTTCC
AGCTATCTGGCCTGGTACCAGCAAAAGCCAGGCCAGGCACCACGA
CTTCTGATTTACGACGCAAGTAACAGGGCGACTGGCATCCCCGCC
CGCTTTTCCGGATCAGGCAGTGGCACAGACTTTACTTTGACGATC
AGTTCTTTGGAGCCAGAGGACTTTGCTGTTTACTACTGTCAGCAG
AGATCCAATTGGCCGATTACCTTCGGCCAAGGTACCAGGCTGGAG
ATAAAGGGTGGCGGCGGCTCTGGTGGAGGTGGTTCCGGTGGGGGA
GGTAGCGAAGTGCAATTGGTGGAAAGCGGTGGTGGCTTGGTTCAA
CCTGGGCGGTCCTTGAGATTGTCATGCGCCGCATCCGGCTTCACC
TTCAACGACTATGCAATGCACTGGGTGCGGCAAGCTCCAGGCAAG
GGCCTTGAGTGGGTCAGCACTATTAGCTGGAACTCCGGGAGTATC
GGATACGCTGATTCAGTCAAAGGTCGATTTACCATTTCCCGCGAT
AATGCCAAAAAAAGCTTGTATCTCCAAATGAACTCACTCAGGGCT
GAAGACACCGCGCTGTATTACTGTGCTAAAGACATCCAATATGGC
AATTACTATTACGGGATGGATGTATGGGGGCAAGGGACAACGGTC
ACTGTCTCATCC
amino acid sequence of CD20-reactive
ScFv1 binding domain
SEQ ID NO: 2
EIVLTQSPATLSLSPGERATLSCRASQSVSSYLAWYQQKPGQAPR
LLIYDASNRATGIPARFSGSGSGTDFTLTISSLEPEDFAVYYCQQ
RSNWPITFGQGTRLEIKGGGGSGGGGSGGGGSEVQLVESGGGLVQ
PGRSLRLSCAASGFTFNDYAMHWVRQAPGKGLEWVSTISWNSGSI
GYADSVKGRFTISRDNAKKSLYLQMNSLRAEDTALYYCAKDIQYG
NYYYGMDVWGQGTTVTVSS
nucleotide sequence of CD 19-reactive
ScFv2 binding domain
SEQ ID NO: 3
GAGGTCCAGCTGGTACAGTCTGGAGCTGAGGTGAAGAAGCCTGGG
GCCTCAGTGAAGGTCTCCTGCAAGGCTTCTGGATACACCTTCACC
AGCTACTATATGCATTGGGTGCGACAGGCCCCTGGACAAGGGCTT
GAGTGGATGGGATTAATCAACCCTAGTGGTGGTAGCACAAGCTAC
GCACAGAAGTTCCAGGGCAGAGTCACCATGACCAGGGACACGTCC
ACGAGCACAGTCTACATGGAGCTGAGCAGCCTGAGATCTGAGGAC
ACGGCCGTGTACTATTGTGCGAGATCGGATCGGGGAATTACCGCC
ACGGACGCTTTTGATATCTGGGGCCAAGGGACAATGGTCACCGTC
TCTTCAGGCGGTGGAGGCTCAGGTGGAGGAGGTAGCGGAGGCGGT
GGTTCCCAGTCTGTGCTGACTCAGCCACCCTCGGTGTCAGTGGCC
CCAGGGCGGATGGCCAAGATTACCTGTGGGGGAAGTGACATTGGA
AATAAAAATGTCCACTGGTATCAGCAGAAGCCTGGCCAGGCTCCT
GTCCTGGTTGTCTATGATGATTACGACCGGCCCTCAGGGATCCCT
GAGCGATTTTCAGGCTCCAACTCTGGGGACGCGGCCACCCTGACG
ATCAGCACGGTCGAAGTCGGGGATGAGGCCGACTATTTCTGTCAG
GTGTGGGACGGTAGTGGTGATCCTTATTGGATGTTCGGCGGAGGG
ACCCAGCTCACCGTTTTAGGT
amino acid sequence of CD19-reactive
ScFv2 binding domain
SEQ ID NO: 4
EVQLVQSGAEVKKPGASVKVSCKASGYTFTSYYMHWVRQAPGQGL
EWMGLINPSGGSTSYAQKFQGRVTMTRDTSTSTVYMELSSLRSED
TAVYYCARSDRGITATDAFDIWGQGTMVTVSSGGGGSGGGGSGGG
GSQSVLTQPPSVSVAPGRMAKITCGGSDIGNKNVHWYQQKPGQAP
VLVVYDDYDRPSGIPERFSGSNSGDAATLTISTVEVGDEADYFCQ
VWDGSGDPYWMFGGGTQLTVLG
nucleotide sequence of CAR D0144
(CD20_CD19 CD8 BBz)
SEQ ID NO: 5
ATGCTGCTGCTGGTGACCAGCCTGCTTCTGTGCGAACTGCCGCAT
CCGGCGTTTCTGTTGATTCCGGAAATTGTGTTGACTCAGTCACCG
GCAACTCTTAGTCTCTCCCCTGGAGAGAGAGCAACACTGAGCTGC
AGAGCATCTCAAAGTGTTTCCAGCTATCTGGCCTGGTACCAGCAA
AAGCCAGGCCAGGCACCACGACTTCTGATTTACGACGCAAGTAAC
AGGGCGACTGGCATCCCCGCCCGCTTTTCCGGATCAGGCAGTGGC
ACAGACTTTACTTTGACGATCAGTTCTTTGGAGCCAGAGGACTTT
GCTGTTTACTACTGTCAGCAGAGATCCAATTGGCCGATTACCTTC
GGCCAAGGTACCAGGCTGGAGATAAAGGGTGGCGGCGGCTCTGGT
GGAGGTGGTTCCGGTGGGGGAGGTAGCGAAGTGCAATTGGTGGAA
AGCGGTGGTGGCTTGGTTCAACCTGGGCGGTCCTTGAGATTGTCA
TGCGCCGCATCCGGCTTCACCTTCAACGACTATGCAATGCACTGG
GTGCGGCAAGCTCCAGGCAAGGGCCTTGAGTGGGTCAGCACTATT
AGCTGGAACTCCGGGAGTATCGGATACGCTGATTCAGTCAAAGGT
CGATTTACCATTTCCCGCGATAATGCCAAAAAAAGCTTGTATCTC
CAAATGAACTCACTCAGGGCTGAAGACACCGCGCTGTATTACTGT
GCTAAAGACATCCAATATGGCAATTACTATTACGGGATGGATGTA
TGGGGGCAAGGGACAACGGTCACTGTCTCATCCGGTGGAGGAGGT
TCAGGCGGAGGAGGCTCAGGCGGAGGCGGTAGCGGCGGAGGAGGA
AGCGGAGGTGGCGGATCAGAGGTCCAGCTGGTACAGTCTGGAGCT
GAGGTGAAGAAGCCTGGGGCCTCAGTGAAGGTCTCCTGCAAGGCT
TCTGGATACACCTTCACCAGCTACTATATGCATTGGGTGCGACAG
GCCCCTGGACAAGGGCTTGAGTGGATGGGATTAATCAACCCTAGT
GGTGGTAGCACAAGCTACGCACAGAAGTTCCAGGGCAGAGTCACC
ATGACCAGGGACACGTCCACGAGCACAGTCTACATGGAGCTGAGC
AGCCTGAGATCTGAGGACACGGCCGTGTACTATTGTGCGAGATCG
GATCGGGGAATTACCGCCACGGACGCTTTTGATATCTGGGGCCAA
GGGACAATGGTCACCGTCTCTTCAGGCGGTGGAGGCTCAGGTGGA
GGAGGTAGCGGAGGCGGTGGTTCCCAGTCTGTGCTGACTCAGCCA
CCCTCGGTGTCAGTGGCCCCAGGGQGGATGGCCAAGATTACCTGT
GGGGGAAGTGACATTGGAÅATAAAAATGTCCACTGGTATCAGCAG
AAGCCTGGCCAGGCTCCTGTCCTGGTTGTCTATGATGATTACGAC
CGGCCCTCAGGGATCCCTGAGCGATTTTCAGGCTCCAACTCTGGG
GACGCGGCCACCCTGACGATCAGCACGGTCGAAGTCGGGGATGAG
GCCGACTATTTCTGTCAGGTGTGGGACGGTAGTGGTGATCCTTAT
TGGATGTTCGGCGGAGGGACCCAGCTCACCGTTTTAGGTGCGGCC
GCAACGACCACTCCTGCACCCCGCCCTCCGACTCCGGCCCCAACC
ATTGCCAGCCAGCCCCTGTCCCTGCGGCCGGAAGCCTGCAGACCG
GCTGCCGGCGGAGCCGTCCATACCCGGGGACTGGATTTCGCCTGC
GATATCTATATCTGGGCACCACTCGCCGGAACCTGTGGAGTGCTG
CTGCTGTCCCTTGTGATCACCCTGTACTGCAAGCGCGGACGGAAG
AAACTCTTGTACATCTTCAAGCAGCCGTTCATGCGCCCTGTGCAA
ACCACCCAAGAAGAGGACGGGTGCTCCTGCCGGTTCCCGGAAGAG
GAAGAGGGCGGCTGCGAACTGCGCGTGAAGTTTTCCCGGTCCGCC
GACGCTCCGGCGTACCAGCAGGGGCAAAACCAGCTGTACAACGAA
CTTAACCTCGGTCGCCGGGAAGAATATGACGTGCTGGACAAGCGG
CGGGGAAGAGATCCCGAGATGGGTGGAAAGCCGCGGCGGAAGAAC
CCTCAGGAGGGCTTGTACAACGAGCTGCAAAAGGACAAAATGGCC
GAAGCCTACTCCGAGATTGGCATGAAGGGAGAGCGCAGACGCGGG
AAGGGACACGATGGACTGTACCAGGGACTGTCAACCGCGACTAAG
GACACTTACGACGCCCTGCACATGCAGGCCCTGCCCCCGCGCTAA
amino acid sequence of CAR D0144
(CD20_CD19 CD8 BBz)
SEQ ID NO: 6
MLLLVTSLLLCELPHPAFLLIPEIVLTQSPATLSLSPGERATLSC
RASQSVSSYLAWYQQKPGQAPRLLIYDASNRATGIPARFSGSGSG
TDFTLTISSLEPEDFAVYYCQQRSNWPITFGQGTRLEIKGGGGSG
GGGSGGGGSEVQLVESGGGLVQPGRSLRLSCAASGFTENDYAMHW
VRQAPGKGLEWVSTISWNSGSIGYADSVKGRFTISRDNAKKSLYL
QMNSLRAEDTALYYCAKDIQYGNYYYGMDVWGQGTTVTVSSGGGG
SGGGGSGGGGSGGGGSGGGGSEVQLVQSGAEVKKPGASVKVSCKA
SGYTFTSYYMHWVRQAPGQGLEWMGLINPSGGSTSYAQKFQGRVT
MTRDTSTSTVYMELSSLRSEDTAVYYCARSDRGITATDAFDIWGQ
GTMVTVSSGGGGSGGGGSGGGGSQSVLTQPPSVSVAPGRMAKITC
GGSDIGNKNVHWYQQKPGQAPVLVVYDDYDRPSGIPERFSGSNSG
DAATLTISTVEVGDEADYFCQVWDGSGDPYWMFGGGTQLTVLGAA
ATTTPAPRPPTPAPTIASQPLSLRPEACRPAAGGAVHTRGLDFAC
DIYIWAPLAGTCGVLLLSLVITLYCKRGRKKLLYIFKQPFMRPVQ
TTQEEDGCSCRFPEEEEGGCELRVKFSRSADAPAYQQGQNQLYNE
LNLGRREEYDVLDKRRGRDPEMGGKPRRKNPQEGLYNELQKDKMA
EAYSEIGMKGERRRGKGHDGLYQGLSTATKDTYDALHMQALPPR
nucleotide sequence of CAR D0255
(CD20_CD19 CD28) CD28 BBz
SEQ ID NO: 7
ATGCTGCTGCTGGTGACCAGCCTGCTTCTGTGCGAACTGCCGCAT
CCGGCGTTTCTGTTGATTCCGGAAATTGTGTTGACTCAGTCACCG
GCAACTCTTAGTCTCTCCCCTGGAGAGAGAGCAACACTGAGCTGC
AGAGCATCTCAAAGTGTTTCCAGCTATCTGGCCTGGTACCAGCAA
AAGCCAGGCCAGGCACCACGACTTCTGATTTACGACGCAAGTAAC
AGGGCGACTGGCATCCCCGCCCGCTTTTCCGGATCAGGCAGTGGC
ACAGACTTTACTTTGACGATCAGTTCTTTGGAGCCAGAGGACTTT
GCTGTTTACTACTGTCAGCAGAGATCCAATTGGCCGATTACCTTC
GGCCAAGGTACCAGGCTGGAGATAAAGGGTGGCGGCGGCTCTGGT
GGAGGTGGTTCCGGTGGGGGAGGTAGCGAAGTGCAATTGGTGGAA
AGCGGTGGTGGCTTGGTTCAACCTGGGCGGTCCTTGAGATTGTCA
TGCGCCGCATCCGGCTTCACCTTCAACGACTATGCAATGCACTGG
GTGCGGCAAGCTCCAGGCAAGGGCCTTGAGTGGGTCAGCACTATT
AGCTGGAACTCCGGGAGTATCGGATACGCTGATTCAGTCAAAGGT
CGATTTACCATTTCCCGCGATAATGCCAAAAAAAGCTTGTATCTC
CAAATGAACTCACTCAGGGCTGAAGACACCGCGCTGTATTACTGT
GCTAAAGACATCCAATATGGCAATTACTATTACGGGATGGATGTA
TGGGGGCAAGGGACAACGGTCACTGTCTCATCCGGTGGAGGAGGT
TCAGGCGGAGGAGGCTCAGGCGGAGGCGGTAGCGGCGGAGGAGGA
AGCGGAGGTGGQGGATCAGAGGTCCAGCTGGTACAGTCTGGAGCT
GAGGTGAAGAAGCCTGGGGCCTCAGTGAAGGTCTCCTGCAAGGCT
TCTGGATACACCTTCACCAGCTACTATATGCATTGGGTGCGACAG
GCCCCTGGACAAGGGCTTGAGTGGATGGGATTAATCAACCCTAGT
GGTGGTAGCACAAGCTACGCACAGAAGTTCCAGGGCAGAGTCACC
ATGACCAGGGACACGTCCACGAGCACAGTCTACATGGAGCTGAGC
AGCCTGAGATCTGAGGACACGGCCGTGTACTATTGTGCGAGATCG
GATCGGGGAATTACCGCCACGGACGCTTTTGATATCTGGGGCCAA
GGGACAATGGTCACCGTCTCTTCAGGCGGTGGAGGCTCAGGTGGA
GGAGGTAGCGGAGGCGGTGGTTCCCAGTCTGTGCTGACTCAGCCA
CCCTCGGTGTCAGTGGCCCCAGGGCGGATGGCCAAGATTACCTGT
GGGGGAAGTGACATTGGAAATAAAAATGTCCACTGGTATCAGCAG
AAGCCTGGCCAGGCTCCTGTCCTGGTTGTCTATGATGATTACGAC
CGGCCCTCAGGGATCCCTGAGCGATTTTCAGGCTCCAACTCTGGG
GACGCGGCCACCCTGACGATCAGCACGGTCGAAGTCGGGGATGAG
GCCGACTATTTCTGTCAGGTGTGGGACGGTAGTGGTGATCCTTAT
TGGATGTTCGGCGGAGGGACCCAGCTCACCGTTTTAGGTGCGGCC
GCAATCGAAGTGATGTATCCACCTCCGTACCTCGATAACGAGAAA
TCAAATGGAACGATCATTCATGTGAAAGGGAAACATCTGTGCCCA
AGCCCATTGTTCCCAGGTCCGTCAAAACCATTCTGGGTGCTTGTC
GTTGTTGGGGGTGTACTCGCATGTTATTCTTTGCTGGTGACTGTG
GCGTTTATCATCTTCTGGGTAAGGAGTAAACGCAGCCGCCTGCTG
CATTCAGACTACATGAACATGACCCCACGGCGGCCCGGCCCAACG
CGCAAACACTACCAACCTTACGCCCCACCGCGAGACTTTGCCGCC
TACAGATCCAAGCGCGGACGGAAGAAACTCTTGTACATCTTCAAG
CAGCCGTTCATGCGCCCTGTGCAAACCACCCAAGAAGAGGACGGG
TGCTCCTGCCGGTTCCCGGAAGAGGAAGAGGGCGGCTGCGAACTG
CGCGTGAAGTTTTCCCGGTCCGCCGACGCTCCGGCGTACCAGCAG
GGGCAAAACCAGCTGTACAACGAACTTAACCTCGGTCGCCGGGAA
GAATATGACGTGCTGGACAAGCGGCGGGGAAGAGATCCCGAGATG
GGTGGAAAGCCGCGGCGGAAGAACCCTCAGGAGGGCTTGTACAAC
GAGCTGCAAAAGGACAAAATGGCCGAAGCCTACTCCGAGATTGGC
ATGAAGGGAGAGCGCAGACGCGGGAAGGGACACGATGGACTGTAC
CAGGGACTGTCAACCGCGACTAAGGACACTTACGACGCCCTGCAC
ATGCAGGCCCTGCCCCCGCGCTAA
amino acid sequence of CAR DO255
(CD20_CD19 CD28) CD28 BBz
SEQ ID NO: 8
MLLLVTSLLLCELPHPAFLLIPEIVLTQSPATLSLSPGERATLSC
RASQSVSSYLAWYQQKPGQAPRLLIYDASNRATGIPARFSGSGSG
TDFTLTISSLEPEDFAVYYCQQRSNWPITFGQGTRLEIKGGGGSG
GGGSGGGGSEVQLVESGGGLVQPGRSLRLSCAASGFTFNDYAMHW
VRQAPGKGLEWVSTISWNSGSIGYADSVKGRFTISRDNAKKSLYL
QMNSLRAEDTALYYCAKDIQYGNYYYGMDVWGQGTTVTVSSGGGG
SGGGGSGGGGSGGGGSGGGGSEVQLVQSGAEVKKPGASVKVSCKA
SGYTFTSYYMHWVRQAPGQGLEWMGLINPSGGSTSYAQKFQGRVT
MTRDTSTSTVYMELSSLRSEDTAVYYCARSDRGITATDAFDIWGQ
GTMVTVSSGGGGSGGGGSGGGGSQSVLTQPPSVSVAPGRMAKITC
GGSDIGNKNVHWYQQKPGQAPVLVVYDDYDRPSGIPERFSGSNSG
DAATLTISTVEVGDEADYFCQVWDGSGDPYWMFGGGTQLTVLGAA
AIEVMYPPPYLDNEKSNGTIIHVKGKHLCPSPLFPGPSKPFWVLV
VVGGVLACYSLLVTVAFIIFWVRSKRSRLLHSDYMNMTPRRPGPT
RKHYQPYAPPRDFAAYRSKRGRKKLLYIFKQPFMRPVQTTQEEDG
CSCRFPEEEEGGCELRVKFSRSADAPAYQQGQNQLYNELNLGRRE
EYDVLDKRRGRDPEMGGKPRRKNPQEGLYNELQKDKMAEAYSEIG
MKGERRRGKGHDGLYQGLSTATKDTYDALHMQALPPR
nucleotide sequence of CAR D0256
(CD20_CD19 CD8 CD28 BBz)
SEQ ID NO: 9
ATGCTGCTGCTGGTGACCAGCCTGCTTCTGTGCGAACTGCCGCAT
CCGGCGTTTCTGTTGATTCCGGAAATTGTGTTGACTCAGTCACCG
GCAACTCTTAGTCTCTCCCCTGGAGAGAGAGCAACACTGAGCTGC
AGAGCATCTCAAAGTGTTTCCAGCTATCTGGCCTGGTACCAGCAA
AAGCCAGGCCAGGCACCACGACTTCTGATTTACGACGCAAGTAAC
AGGGCGACTGGCATCCCCGCCCGCTTTTCCGGATCAGGCAGTGGC
ACAGACTTTACTTTGACGATCAGTTCTTTGGAGCCAGAGGACTTT
GCTGTTTACTACTGTCAGCAGAGATCCAATTGGCCGATTACCTTC
GGCCAAGGTACCAGGCTGGAGATAAAGGGTGGCGGCGGCTCTGGT
GGAGGTGGTTCCGGTGGGGGAGGTAGCGAAGTGCAATTGGTGGAA
AGCGGTGGTGGCTTGGTTCAACCTGGGCGGTCCTTGAGATTGTCA
TGCGCCGCATCCGGCTTCACCTTCAACGACTATGCAATGCACTGG
GTGCGGCAAGCTCCAGGCAAGGGCCTTGAGTGGGTCAGCACTATT
AGCTGGAACTCCGGGAGTATCGGATACGCTGATTCAGTCAAAGGT
CGATTTACCATTTCCCGCGATAATGCCAAAAAAAGCTTGTATCTC
CAAATGAACTCACTCAGGGCTGAAGACACCGCGCTGTATTACTGT
GCTAAAGACATCCAATATGGCAATTACTATTACGGGATGGATGTA
TGGGGGCAAGGGACAACGGTCACTGTCTCATCCGGTGGAGGAGGT
TCAGGCGGAGGAGGCTCAGGCGGAGGCGGTAGCGGCGGAGGAGGA
AGCGGAGGTGGCGGATCAGAGGTCCAGCTGGTACAGTCTGGAGCT
GAGGTGAAGAAGCCTGGGGCCTCAGTGAAGGTCTCCTGCAAGGCT
TCTGGATACACCTTCACCAGCTACTATATGCATTGGGTGCGACAG
GCCCCTGGACAAGGGCTTGAGTGGATGGGATTAATCAACCCTAGT
GGTGGTAGCACAAGCTACGCACAGAAGTTCCAGGGCAGAGTCACC
ATGACCAGGGACACGTCCACGAGCACAGTCTACATGGAGCTGAGC
AGCCTGAGATCTGAGGACACGGCCGTGTACTATTGTGCGAGATCG
GATCGGGGAATTACCGCCACGGACGCTTTTGATATCTGGGGCCAA
GGGACAATGGTCACCGTCTCTTCAGGCGGTGGAGGCTCAGGTGGA
GGAGGTAGCGGAGGCGGTGGTTCCCAGTCTGTGCTGACTCAGCCA
CCCTCGGTGTCAGTGGCCCCAGGGCGGATGGCCAAGATTACCTGT
GGGGGAAGTGACATTGGAAATAAAAATGTCCACTGGTATCAGCAG
AAGCCTGGCCAGGCTCCTGTCCTGGTTGTCTATGATGATTACGAC
CGGCCCTCAGGGATCCCTGAGCGATTTTCAGGCTCCAACTCTGGG
GACGCGGCCACCCTGACGATCAGCACGGTCGAAGTCGGGGATGAG
GCCGACTATTTCTGTCAGGTGTGGGACGGTAGTGGTGATCCTTAT
TGGATGTTCGGCGGAGGGACCCAGCTCACCGTTTTAGGTGCGGCC
GCGACTACCACTCCTGCACCACGGCCACCTACCCCAGCCCCCACC
ATTGCAAGCCAGCCACTTTCACTGCGCCCCGAAGCGTGTAGACCA
GCTGCTGGAGGAGCCGTGCATACCCGAGGGCTGGACTTCGCCTGT
GACATCTACATCTGGGCCCCATTGGCTGGAACTTGCGGCGTGCTG
CTCTTGTCTCTGGTCATTACCCTGTACTGCAGGAGTAAACGCAGC
CGCCTGCTGCATTCAGACTACATGAACATGACCCCACGGCGGCCC
GGCCCAACGCGCAAACACTACCAACCTTACGCCCCACCGCGAGAC
TTTGCCGCCTACAGATCCAAGCGCGGACGGAAGAAACTCTTGTAC
ATCTTCAAGCAGCCGTTCATGCGCCCTGTGCAAACCACCCAAGAA
GAGGACGGGTGCTCCTGCCGGTTCCCGGAAGAGGAAGAGGGCGGC
TGCGAACTGCGCGTGAAGTTTTCCCGGTCCGCCGACGCTCCGGCG
TACCAGCAGGGGCAAAACCAGCTGTACAACGAACTTAACCTCGGT
CGCCGGGAAGAATATGACGTGCTGGACAAGCGGCGGGGAAGAGAT
CCCGAGATGGGTGGAAAGCCGCGGCGGAAGAACCCTCAGGAGGGC
TTGTACAACGAGCTGCAAAAGGACAAAATGGCCGAAGCCTACTCC
GAGATTGGCATGAAGGGAGAGCGCAGACGCGGGAAGGGACACGAT
GGACTGTACCAGGGACTGTCAACCGCGACTAAGGACACTTACGAC
GCCCTGCACATGCAGGCCCTGCCCCCGCGCTAA
amino acid sequence of CAR D0256
(CD20_CD19 CD8 CD28 BBz)
SEQ ID NO: 10
MLLLVTSLLLCELPHPAFLLIPEIVLTQSPATLSLSPGERATLSC
RASQSVSSYLAWYQQKPGQAPRLLIYDASNRATGIPARFSGSGSG
TDFTLTISSLEPEDFAVYYCQQRSNWPITFGQGTRLEIKGGGGSG
GGGSGGGGSEVQLVESGGGLVQPGRSLRLSCAASGFTENDYAMHW
VRQAPGKGLEWVSTISWNSGSIGYADSVKGRFTISRDNAKKSLYL
QMNSLRAEDTALYYCAKDIQYGNYYYGMDVWGQGTTVTVSSGGGG
SGGGGSGGGGSGGGGSGGGGSEVQLVQSGAEVKKPGASVKVSCKA
SGYTFTSYYMHWVRQAPGQGLEWMGLINPSGGSTSYAQKFQGRVT
MTRDTSTSTVYMELSSLRSEDTAVYYCARSDRGITATDAFDIWGQ
GTMVTVSSGGGGSGGGGSGGGGSQSVLTQPPSVSVAPGRMAKITC
GGSDIGNKNVHWYQQKPGQAPVLVVYDDYDRPSGIPERFSGSNSG
DAATLTISTVEVGDEADYFCQVWDGSGDPYWMFGGGTQLTVLGAA
ATTTPAPRPPTPAPTIASQPLSLRPEACRPAAGGAVHTRGLDFAC
DIYIWAPLAGTCGVLLLSLVITLYCRSKRSRLLHSDYMNMTPRRP
GPTRKHYQPYAPPRDFAAYRSKRGRKKLLYIFKQPFMRPVQTTQE
EDGCSCRFPEEEEGGCELRVKFSRSADAPAYQQGQNQLYNELNLG
RREEYDVLDKRRGRDPEMGGKPRRKNPQEGLYNELQKDKMAEAYS
EIGMKGERRRGKGHDGLYQGLSTATKDTYDALHMQALPPR
nucleotide sequence of leader/signal
peptide sequence (LP)
SEQ ID NO: 11
atgctgctgctggtgaccagcctgctgctgtgcgaactgccgcat
ccggcgtttctgctgattccg
amino acid sequence of leader/signal
peptide sequence (LP)
SEQ ID NO: 12
MLLLVTSLLLCELPHPAFLLIP
nucleotide sequence of CAR D0257
(CD20 CD8 CD28z_CD19 CD8 BBz)
SEQ ID NO: 13
ATGCTGCTGCTGGTGACCAGCCTGCTTCTGTGCGAACTGCCGCAT
CCGGCGTTTCTGTTGATTCCGGAAATTGTGTTGACTCAGTCACCG
GCAACTCTTAGTCTCTCCCCTGGAGAGAGAGCAACACTGAGCTGC
AGAGCATCTCAAAGTGTTTCCAGCTATCTGGCCTGGTACCAGCAA
AAGCCAGGCCAGGCACCACGACTTCTGATTTACGACGCAAGTAAC
AGGGCGACTGGCATCCCCGCCCGCTTTTCCGGATCAGGCAGTGGC
ACAGACTTTACTTTGACGATCAGTTCTTTGGAGCCAGAGGACTTT
GCTGTTTACTACTGTCAGCAGAGATCCAATTGGCCGATTACCTTC
GGCCAAGGTACCAGGCTGGAGATAAAGGGTGGCGGCGGCTCTGGT
GGAGGTGGTTCCGGTGGGGGAGGTAGCGAAGTGCAATTGGTGGAA
AGCGGTGGTGGCTTGGTTCAACCTGGGCGGTCCTTGAGATTGTCA
TGCGCCGCATCCGGCTTCACCTTCAACGACTATGCAATGCACTGG
GTGCGGCAAGCTCCAGGCAAGGGCCTTGAGTGGGTCAGCACTATT
AGCTGGAACTCCGGGAGTATCGGATACGCTGATTCAGTCAAAGGT
CGATTTACCATTTCCCGCGATAATGCCAAAAAAAGCTTGTATCTC
CAAATGAACTCACTCAGGGCTGAAGACACCGCGCTGTATTACTGT
GCTAAAGACATCCAATATGGCAATTACTATTACGGGATGGATGTA
TGGGGGCAAGGGACAACGGTCACTGTCTCATCCGCGGCCGCGACT
ACCACTCCTGCACCACGGCCACCTACCCCAGCCCCCACCATTGCA
AGCCAGCCACTTTCACTGCGCCCCGAAGCGTGTAGACCAGCTGCT
GGAGGAGCCGTGCATACCCGAGGGCTGGACTTCGCCTGTGACATC
TACATCTGGGCCCCATTGGCTGGAACTTGCGGCGTGCTGCTCTTG
TCTCTGGTCATTACCCTGTACTGCCGGTCGAAGAGGTCCAGACTC
TTGCACTCCGACTACATGAACATGACTCCTAGAAGGCCCGGACCC
ACTAGAAAGCACTACCAGCCGTACGCCCCTCCTCGGGATTTCGCC
GCATACCGGTCCAGAGTGAAGTTCAGCCGCTCAGCCGATGCACCG
GCCTACCAGCAGGGACAGAACCAGCTCTACAACGAGCTCAACCTG
GGTCGGCGGGAAGAATATGACGTGCTGGACAAACGGCGCGGCAGA
GATCCGGAGATGGGGGGAAAGCCGAGGAGGAAGAACCCTCAAGAG
GGCCTGTACAACGAACTGCAGAAGGACAAGATGGCGGAAGCCTAC
TCCGAGATCGGCATGAAGGGAGAACGCCGGAGAGGGAAGGGTCAT
GACGGACTGTACCAGGGCCTGTCAACTGCCACTAAGGACACTTAC
GATGCGCTCCATATGCAAGCTTTGCCCCCGCGGCGCGCGAAACGC
GGCAGCGGCGCGACCAACTTTAGCCTGCTGAAACAGGCGGGCGAT
GTGGAAGAAAACCCGGGCCCGCGAGCAAAGAGGAATATTATGGCT
CTGCCTGTTACGGCACTGCTCCTTCCGCTTGCATTGTTGTTGCAC
GCAGCGCGGCCCGAGGTCCAGCTGGTACAGTCTGGAGCTGAGGTG
AAGAAGCCTGGGGCCTCAGTGAAGGTCTCCTGCAAGGCTTCTGGA
TACACCTTCACCAGCTACTATATGCACTGGGTGCGACAGGCCCCT
GGACAAGGGCTTGAGTGGATGGGATTAATCAACCCTAGTGGTGGT
AGCACAAGCTACGCACAGAAGTTCCAGGGCAGAGTCACCATGACC
AGGGACACGTCCACGAGCACAGTCTACATGGAGCTGAGCAGCCTG
AGATCTGAGGACACGGCCGTGTATTACTGTGCGAGATCGGATCGG
GGAATTACCGCCACGGACGCTTTTGATATCTGGGGCCAAGGGACA
ATGGTCACCGTCTCTTCAGGCGGAGGAGGCTCCGGGGGAGGAGGT
TCCGGGGGCGGGGGTTCCCAGTCTGTGCTGACTCAGCCACCCTCG
GTGTCAGTGGCCCCAGGGCGGATGGCCAAGATTACCTGTGGGGGA
AGTGACATTGGAAATAAAAATGTCCACTGGTATCAGCAGAAGCCA
GGCCAGGCCCCTGTCCTGGTTGTCTATGATGATTACGACCGGCCC
TCAGGGATCCCTGAGCGATTCTCTGGCTCCAACTCTGGGGACGCG
GCCACCCTGACGATCAGCACGGTCGAAGTCGGGGATGAGGCCGAC
TATTTCTGTCAGGTGTGGGACGGTAGTGGTGATCCTTATTGGATG
TTCGGCGGAGGGACCCAGCTCACCGTTTTAGGTGCTAGCGCAACC
ACTACGCCTGCTCCGCGGCCTCCAACGCCCGCGCCCACGATAGCT
AGTCAGCCGTTGTCTCTCCGACCAGAGGCGTGTAGACCGGCCGCT
GGCGGAGCCGTACATACTCGCGGACTCGACTTCGCTTGCGACATC
TACATTTGGGCACCCTTGGCTGGGACCTGTGGGGTGCTGTTGCTG
TCCTTGGTTATTACGTTGTACTGCAAGAGGGGCCGGAAGAAGCTG
CTTTACATCTTCAAGCAGCCGTTCATGCGGCCCGTGCAGACGACT
CAGGAAGAGGACGGATGCTCGTGCAGATTCCCTGAGGAGGAAGAG
GGGGGATGCGAACTGAGAGTCAAATTTTCCAGGTCCGCAGATGCC
CCCGCGTACCAGCAAGGCCAGAACCAACTTTACAACGAACTGAAC
CTGGGTCGCCGGGAGGAATATGATGTGCTGGATAAACGAAGGGGG
AGGGACCCTGAGATGGGAGGGAAACCTCGCAGGAAAAACCCGCAG
GAAGGTTTGTACAACGAGTTGCAGAAGGATAAGATGGCTGAGGCT
TACTCTGAAATAGGGATGAAGGGAGAGAGACGGAGAGGAAAAGGC
CATGATGGCCTTTACCAGGGCTTGAGCACAGCAACAAAGGATACT
TACGACGCTCTTCACATGCAAGCTCTGCCACCACGGTAG
amino acid sequence of CAR D0257
(CD20 CD8 CD28z_CD19 CD8 BBz)
SEQ ID NO: 14
MLLLVTSLLLCELPHPAFLLIPEIVLTQSPATLSLSPGERATLSC
RASQSVSSYLAWYQQKPGQAPRLLIYDASNRATGIPARFSGSGSG
TDFTLTISSLEPEDFAVYYCQQRSNWPITFGQGTRLEIKGGGGSG
GGGSGGGGSEVQLVESGGGLVQPGRSLRLSCAASGFTFNDYAMHW
VRQAPGKGLEWVSTISWNSGSIGYADSVKGRFTISRDNAKKSLYL
QMNSLRAEDTALYYCAKDIQYGNYYYGMDVWGQGTTVTVSSAAAT
TTPAPRPPTPAPTIASQPLSLRPEACRPAAGGAVHTRGLDFACDI
YIWAPLAGTCGVLLLSLVITLYCRSKRSRLLHSDYMNMTPRRPGP
TRKHYQPYAPPRDFAAYRSRVKFSRSADAPAYQQGQNQLYNELNL
GRREEYDVLDKRRGRDPEMGGKPRRKNPQEGLYNELQKDKMAEAY
SEIGMKGERRRGKGHDGLYQGLSTATKDTYDALHMQALPPRRAKR
GSGATNFSLLKQAGDVEENPGPRAKRNIMALPVTALLLPLALLLH
AARPEVQLVQSGAEVKKPGASVKVSCKASGYTFTSYYMHWVRQAP
GQGLEWMGLINPSGGSTSYAQKFQGRVTMTRDTSTSTVYMELSSL
RSEDTAVYYCARSDRGITATDAFDIWGQGTMVTVSSGGGGSGGGG
SGGGGSQSVLTQPPSVSVAPGRMAKITCGGSDIGNKNVHWYQQKP
GQAPVLVVYDDYDRPSGIPERFSGSNSGDAATLTISTVEVGDEAD
YFCQVWDGSGDPYWMFGGGTQLTVLGASATTTPAPRPPTPAPTIA
SQPLSLRPEACRPAAGGAVHTRGLDFACDIYIWAPLAGTCGVLLL
SLVITLYCKRGRKKLLYIFKQPFMRPVQTTQEEDGCSCRFPEEEE
GGCELRVKFSRSADAPAYQQGQNQLYNELNLGRREEYDVLDKRRG
RDPEMGGKPRRKNPQEGLYNELQKDKMAEAYSEIGMKGERRRGKG
HDGLYQGLSTATKDTYDALHMQALPPR
nucleotide sequence of CAR D0258
(CD20 CD8 BBz_CD19 CD8 CD28z)
SEQ ID NO: 15
ATGCTGCTGCTGGTGACCAGCCTGCTTCTGTGCGAACTGCCGCAT
CCGGCGTTTCTGTTGATTCCGGAAATTGTGTTGACTCAGTCACCG
GCAACTCTTAGTCTCTCCCCTGGAGAGAGAGCAACACTGAGCTGC
AGAGCATCTCAAAGTGTTTCCAGCTATCTGGCCTGGTACCAGCAA
AAGCCAGGCCAGGCACCACGACTTCTGATTTACGACGCAAGTAAC
AGGGCGACTGGCATCCCCGCCCGCTTTTCCGGATCAGGCAGTGGC
ACAGACTTTACTTTGACGATCAGTTCTTTGGAGCCAGAGGACTTT
GCTGTTTACTACTGTCAGCAGAGATCCAATTGGCCGATTACCTTC
GGCCAAGGTACCAGGCTGGAGATAAAGGGTGGCGGCGGCTCTGGT
GGAGGTGGTTCCGGTGGGGGAGGTAGCGAAGTGCAATTGGTGGAA
AGCGGTGGTGGCTTGGTTCAACCTGGGCGGTCCTTGAGATTGTCA
TGCGCCGCATCCGGCTTCACCTTCAACGACTATGCAATGCACTGG
GTGCGGCAAGCTCCAGGCAAGGGCCTTGAGTGGGTCAGCACTATT
AGCTGGAACTCCGGGAGTATCGGATACGCTGATTCAGTCAAAGGT
CGATTTACCATTTCCCGCGATAATGCCAAAAAAAGCTTGTATCTC
CAAATGAACTCACTCAGGGCTGAAGACACCGCGCTGTATTACTGT
GCTAAAGACATCCAATATGGCAATTACTATTACGGGATGGATGTA
TGGGGGCAAGGGACAACGGTCACTGTCTCATCCGCGGCCGCAACG
ACCACTCCTGCACCACGGCCACCTACCCCAGCCCCCACCATTGCA
AGCCAGCCACTTTCACTGCGCCCCGAAGCGTGTAGACCAGCTGCT
GGAGGAGCCGTGCATACCCGAGGGCTGGACTTCGCCTGTGACATC
TACATCTGGGCCCCATTGGCTGGAACTTGCGGCGTGCTGCTCTTG
TCTCTGGTCATTACCCTGTACTGCAAGCGCGGACGGAAGAAACTC
TTGTACATCTTCAAGCAGCCGTTCATGCGCCCTGTGCAAACCACC
CAAGAAGAGGACGGGTGCTCCTGCCGGTTCCCGGAAGAGGAAGAG
GGCGGCTGCGAACTGAGAGTGAAGTTTAGCCGCTCAGCCGATGCA
CCGGCCTACCAGCAGGGACAGAACCAGCTCTACAACGAGCTCAAC
CTGGGTCGGCGGGAAGAATATGACGTGCTGGACAAACGGCGCGGC
AGAGATCCGGAGATGGGGGGAAAGCCGAGGAGGAAGAACCCTCAA
GAGGGCCTGTACAACGAACTGCAGAAGGACAAGATGGCGGAAGCC
TACTCCGAGATCGGCATGAAGGGAGAACGCCGGAGAGGGAAGGGT
CATGACGGACTGTACCAGGGCCTGTCAACTGCCACTAAGGACACT
TACGATGCGCTCCATATGCAAGCTTTGCCCCCGCGGCGCGCGAAA
CGCGGCAGCGGCGCGACCAACTTTAGCCTGCTGAAACAGGGGGCG
ATGTGGAAGAAAACCCGGGCCCGCGAGCAAAGAGGAATATTATGG
CTCTGCCTGTTACGGCACTGCTCCTTCCGCTTGCATTGTTGTTGC
ACGCAGCGCGGCCCGAGGTCCAGCTGGTACAGTCTGGAGCTGAGG
TGAAGAAGCCTGGGGCCTCAGTGAAGGTCTCCTGCAAGGCTTCTG
GATACACCTTCACCAGCTACTATATGCACTGGGTGCGACAGGCCC
CTGGACAAGGGCTTGAGTGGATGGGATTAATCAACCCTAGTGGTG
GTAGCACAAGCTACGCACAGAAGTTCCAGGGCAGAGTCACCATGA
CCAGGGACACGTCCACGAGCACAGTCTACATGGAGCTGAGCAGCC
TGAGATCTGAGGACACGGCCGTGTATTACTGTGCGAGATCGGATC
GGGGAATTACCGCCACGGACGCTTTTGATATCTGGGGCCAAGGGA
CAATGGTCACCGTCTCTTCAGGCGGAGGAGGCTCCGGGGGAGGAG
GTTCCGGGGGCGGGGGTTCCCAGTCTGTGCTGACTCAGCCACCCT
CGGTGTCAGTGGCCCCAGGGCGGATGGCCAAGATTACCTGTGGGG
GAAGTGACATTGGAAATAAAAATGTCCACTGGTATCAGCAGAAGC
CAGGCCAGGCCCCTGTCCTGGTTGTCTATGATGATTACGACCGGC
CCTCAGGGATCCCTGAGCGATTCTCTGGCTCCAACTCTGGGGACG
CGGCCACCCTGACGATCAGCACGGTCGAAGTCGGGGATGAGGCCG
ACTATTTCTGTCAGGTGTGGGACGGTAGTGGTGATCCTTATTGGA
TGTTCGGCGGAGGGACCCAGCTCACCGTTTTAGGTGCTAGCGCAA
CCACTACGCCTGCTCCGCGGCCTCCAACGCCCGCGCCCACGATAG
CTAGTCAGCCGTTGTCTCTCCGACCAGAGGCGTGTAGACCGGCCG
CTGGCGGAGCCGTACATACTCGCGGACTCGACTTCGCTTGCGACA
TCTACATTTGGGCACCCTTGGCTGGGACCTGTGGGGTGCTGTTGC
TGTCCTTGGTTATTACGTTGTACTGCCGGTCGAAGAGGTCCAGAC
TCTTGCACTCCGACTACATGAACATGACTCCTAGAAGGCCCGGAC
CCACTAGAAAGCACTACCAGCCGTACGCCCCTCCTCGGGATTTCG
CCGCATACCGGTCCAGAGTCAAATTTTCCAGGTCCGCAGATGCCC
CCGCGTACCAGCAAGGCCAGAACCAACTTTACAACGAACTGAACC
TGGGTCGCCGGGAGGAATATGATGTGCTGGATAAACGAAGGGGGA
GGGACCCTGAGATGGGAGGGAAACCTCGCAGGAAAAACCCGCAGG
AAGGTTTGTACAACGAGTTGCAGAAGGATAAGATGGCTGAGGCTT
ACTCTGAAATAGGGATGAAGGGAGAGAGACGGAGAGGAAAAGGCC
ATGATGGCCTTTACCAGGGCTTAAGCACAGCAACAAAGGATACTT
ACGACGCTCTTCACATGCAAGCTCTGCCACCACGGTAG
amino acid sequence of CAR D0258
(CD20 CD8 BBz_CD19 CD8 CD28z)
SEQ ID NO: 16
MLLLVTSLLLCELPHPAFLLIPEIVLTQSPATLSLSPGERATLSC
RASQSVSSYLAWYQQKPGQAPRLLIYDASNRATGIPARFSGSGSG
TDFTLTISSLEPEDFAVYYCQQRSNWPITFGQGTRLEIKGGGGGG
GGSGGGGSEVQLVESGGGLVQPGRSLRLSCAASGFTENDYAMHWV
RQAPGKGLEWVSTISWNSGSIGYADSVKGRFTISRDNAKKSLYLQ
MNSLRAEDTALYYCAKDIQYGNYYYGMDVWGQGTTVTVSSAAATT
TPAPRPPTPAPTIASQPLSLRPEACRPAAGGAVHTRGLDFACDIY
IWAPLAGTCGVLLLSLVITLYCKRGRKKLLYIFKQPFMRPVQTTQ
EEDGCSCRFPEEEEGGCELRVKFSRSADAPAYQQGQNQLYNELNL
GRREEYDVLDKRRGRDPEMGGKPRRKNPQEGLYNELQKDKMAEAY
SEIGMKGERRRGKGHDGLYQGLSTATKDTYDALHMQALPPRRAKR
GSGATNFSLLKQAGDVEENPGPRAKRNIMALPVTALLLPLALLLH
AARPEVQLVQSGAEVKKPGASVKVSCKASGYTFTSYYMHWVRQAP
GQGLEWMGLINPSGGSTSYAQKFQGRVTMTRDTSTSTVYMELSSL
RSEDTAVYYCARSDRGITATDAFDIWGQGTMVTVSSGGGGSGGGG
SGGGGSQSVLTQPPSVSVAPGRMAKITCGGSDIGNKNVHWYQQKP
GQAPVLVVYDDYDRPSGIPERFSGSNSGDAATLTISTVEVGDEAD
YFCQVWDGSGDPYWMFGGGTQLTVLGASATTTPAPRPPTPAPTIA
SQPLSLRPEACRPAAGGAVHTRGLDFACDIYIWAPLAGTCGVLLL
SLVITLYCRSKRSRLLHSDYMNMTPRRPGPTRKHYQPYAPPRDFA
AYRSRVKFSRSADAPAYQQGQNQLYNELNLGRREEYDVLDKRRGR
DPEMGGKPRRKNPQEGLYNELQKDKMAEAYSEIGMKGERRRGKGH
DGLYQGLSTATKDTYDALHMQALPPR
nucleotide sequence of CAR D0266
(CD20 CD8 OX40 OX40z_CD19 CD8 ICOSz)
SEQ ID NO: 17
ATGCTGCTGCTGGTGACCAGCCTGCTTCTGTGCGAACTGCCGCAT
CCGGCGTTTCTGTTGATTCCGGAAATTGTGTTGACTCAGTCACCG
GCAACTCTTAGTCTCTCCCCTGGAGAGAGAGCAACACTGAGCTGC
AGAGCATCTCAAAGTGTTTCCAGCTATCTGGCCTGGTACCAGCAA
AAGCCAGGCCAGGCACCACGACTTCTGATTTACGACGCAAGTAAC
AGGGCGACTGGCATCCCCGCCCGCTTTTCCGGATCAGGCAGTGGC
ACAGACTTTACTTTGACGATCAGTTCTTTGGAGCCAGAGGACTTT
GCTGTTTACTACTGTCAGCAGAGATCCAATTGGCCGATTACCTTC
GGCCAAGGTACCAGGCTGGAGATAAAGGGTGGCGGCGGCTCTGGT
GGAGGTGGTTCCGGTGGGGGAGGTAGCGAAGTGCAATTGGTGGAA
AGCGGTGGTGGCTTGGTTCAACCTGGGCGGTCCTTGAGATTGTCA
TGCGCCGCATCCGGCTTCACCTTCAACGACTATGCAATGCACTGG
GTGCGGCAAGCTCCAGGCAAGGGCCTTGAGTGGGTCAGCACTATT
AGCTGGAACTCCGGGAGTATCGGATACGCTGATTCAGTCAAAGGT
CGATTTACCATTTCCCGCGATAATGCCAAAAAAAGCTTGTATCTC
CAAATGAACTCACTCAGGGCTGAAGACACCGCGCTGTATTACTGT
GCTAAAGACATCCAATATGGCAATTACTATTACGGGATGGATGTA
TGGGGGCAAGGGACAACGGTCACTGTCTCATCCGCGGCCGCAACG
ACCACTCCAGCACCGAGACCGCCAACCCCCGCGCCTACCATCGCA
AGTCAACCACTTTCTCTCAGGCCTGAAGCGTGCCGACCTGCAGCT
GGTGGGGCAGTACATACCAGGGGTTTGGACTTCGCATGTGACGTG
GCGGCAATTCTCGGCCTGGGACTTGTCCTTGGTCTGCTTGGTCCG
CTCGCAATACTTCTGGCCTTGTACCTGCTCCGCAGAGACCAAAGA
CTTCCGCCCGACGCCCACAAGCCCCCAGGAGGAGGTTCCTTCAGA
ACGCCTATACAAGAAGAACAAGCAGATGCCCACTCTACCCTGGCT
AAAATCAGGGTGAAGTTTAGCCGCTCAGCCGATGCACCGGCCTAC
CAGCAGGGACAGAACCAGCTCTACAACGAGCTCAACCTGGGTCGG
CGGGAAGAATATGACGTGCTGGACAAACGGCGCGGCAGAGATCCG
GAGATGGGGGGAAAGCCGAGGAGGAAGAACCCTCAAGAGGGCCTG
TACAACGAACTGCAGAAGGACAAGATGGCGGAAGCCTACTCCGAG
ATCGGCATGAAGGGAGAACGCCGGAGAGGGAAGGGTCATGACGGA
CTGTACCAGGGCCTGTCAACTGCCACTAAGGACACTTACGATGCG
CTCCATATGCAAGCTTTGCCCCCGCGGCGCGCGAAACGCGGCAGC
GGCGCGACCAACTTTAGCCTGCTGAAACAGGGGGCGATGTGGAAG
AAAACCCGGGCCCGCGAGCAAAGAGGAATATTATGGCTCTGCCTG
TTACGGCACTGCTCCTTCCGCTTGCATTGTTGTTGCACGCAGCGC
GGCCCGAGGTCCAGCTGGTACAGTCTGGAGCTGAGGTGAAGAAGC
CTGGGGCCTCAGTGAAGGTCTCCTGCAAGGCTTCTGGATACACCT
TCACCAGCTACTATATGCACTGGGTGCGACAGGCCCCTGGACAAG
GGCTTGAGTGGATGGGATTAATCAACCCTAGTGGTGGTAGCACAA
GCTACGCACAGAAGTTCCAGGGCAGAGTCACCATGACCAGGGACA
CGTCCACGAGCACAGTCTACATGGAGCTGAGCAGCCTGAGATCTG
AGGACACGGCCGTGTATTACTGTGCGAGATCGGATCGGGGAATTA
CCGCCACGGACGCTTTTGATATCTGGGGCCAAGGGACAATGGTCA
CCGTCTCTTCAGGCGGAGGAGGCTCCGGGGGAGGAGGTTCCGGGG
GCGGGGGTTCCCAGTCTGTGCTGACTCAGCCACCCTCGGTGTCAG
TGGCCCCAGGGCGGATGGCCAAGATTACCTGTGGGGGAAGTGACA
TTGGAAATAAAAATGTCCACTGGTATCAGCAGAAGCCAGGCCAGG
CCCCTGTCCTGGTTGTCTATGATGATTACGACCGGCCCTCAGGGA
TCCCTGAGCGATTCTCTGGCTCCAACTCTGGGGACGCGGCCACCC
TGACGATCAGCACGGTCGAAGTCGGGGATGAGGCCGACTATTTCT
GTCAGGTGTGGGACGGTAGTGGTGATCCTTATTGGATGTTCGGCG
GAGGGACCCAGCTCACCGTTTTAGGTGCTAGCGCAACCACTACGC
CTGCTCCGCGGCCTCCAACGCCCGCGCCCACGATAGCTAGTCAGC
CGTTGTCTCTCCGACCAGAGGCGTGTAGACCGGCCGCTGGCGGAG
CCGTACATACTCGCGGACTCGACTTCGCTTGCGACATCTACATTT
GGGCACCCTTGGCTGGGACCTGTGGGGTGCTGTTGCTGTCCTTGG
TTATTACGTTGTACTGCTGGCTGACAAAAAAGAAGTATTCATCTA
GTGTACATGATCCGAACGGTGAATACATGTTCATGCGCGCGGTGA
ACACGGCCAAGAAGAGCAGACTGACCGACGTAACCCTTAGAGTCA
AATTTTCCAGGTCCGCAGATGCCCCCGCGTACCAGCAAGGCCAGA
ACCAACTTTACAACGAACTGAACCTGGGTCGCCGGGAGGAATATG
ATGTGCTGGATAAACGAAGGGGGAGGGACCCTGAGATGGGAGGGA
AACCTCGCAGGAAAAACCCGCAGGAAGGTTTGTACAACGAGTTGC
AGAAGGATAAGATGGCTGAGGCTTACTCTGAAATAGGGATGAAGG
GAGAGAGACGGAGAGGAAAAGGCCATGATGGCCTTTACCAGGGCT
TGAGCACAGCAACAAAGGATACTTACGACGCTCTTCACATGCAAG
CTCTGCCACCACGGTAG
amino acid sequence of CAR D0266
(CD20 CD8 OX40 OX40z_CD19 CD8 ICOSz)
SEQ ID NO: 18
MLLLVTSLLLCELPHPAFLLIPEIVLTQSPATLSLSPGERATLSC
RASQSVSSYLAWYQQKPGQAPRLLIYDASNRATGIPARFSGSGSG
TDFTLTISSLEPEDFAVYYCQQRSNWPITFGQGTRLEIKGGGGSG
GGGSGGGGSEVQLVESGGGLVQPGRSLRLSCAASGFTENDYAMHW
VRQAPGKGLEWVSTISWNSGSIGYADSVKGRFTISRDNAKKSLYL
QMNSLRAEDTALYYCAKDIQYGNYYYGMDVWGQGTTVTVSSAAAT
TTPAPRPPTPAPTIASQPLSLRPEACRPAAGGAVHTRGLDFACDV
AAILGLGLVLGLLGPLAILLALYLLRRDQRLPPDAHKPPGGGSFR
TPIQEEQADAHSTLAKIRVKFSRSADAPAYQQGQNQLYNELNLGR
REEYDVLDKRRGRDPEMGGKPRRKNPQEGLYNELQKDKMAEAYSE
IGMKGERRRGKGHDGLYQGLSTATKDTYDALHMQALPPRRAKRGS
GATNFSLLKQAGDVEENPGPRAKRNIMALPVTALLLPLALLLHAA
RPEVQLVQSGAEVKKPGASVKVSCKASGYTFTSYYMHWVRQAPGQ
GLEWMGLINPSGGSTSYAQKFQGRVTMTRDTSTSTVYMELSSLRS
EDTAVYYCARSDRGITATDAFDIWGQGTMVTVSSGGGGSGGGGSG
GGGSQSVLTQPPSVSVAPGRMAKITCGGSDIGNKNVHWYQQKPGQ
APVLVVYDDYDRPSGIPERFSGSNSGDAATLTISTVEVGDEADYF
CQVWDGSGDPYWMFGGGTQLTVLGASATTTPAPRPPTPAPTIASQ
PLSLRPEACRPAAGGAVHTRGLDFACDIYIWAPLAGTCGVLLLSL
VITLYCWLTKKKYSSSVHDPNGEYMFMRAVNTAKKSRLTDVTLRV
KFSRSADAPAYQQGQNQLYNELNLGRREEYDVLDKRRGRDPEMGG
KPRRKNPQEGLYNELQKDKMAEAYSEIGMKGERRRGKGHDGLYQG
LSTATKDTYDALHMQALPPR
nucleotide sequence of CAR LTG1497
(mCD20_CD19 CD8 BBz)
SEQ ID NO: 19
ATGCTCCTTCTCGTGACCTCCCTGCTTCTCTGCGAACTGCCCCAT
CCTGCCTTCCTGCTGATTCCCGAGGTGCAGTTGCAACAGTCAGGA
GCTGAACTGGTCAAGCCAGGAGCCAGCGTGAAGATGAGCTGCAAG
GCCTCCGGTTACACCTTCACCTCCTACAACATGCACTGGGTGAAA
CAGACCCCGGGACAAGGGCTCGAATGGATTGGCGCCATCTACCCC
GGGAATGGCGATACTTCGTACAACCAGAAGTTCAAGGGAAAGGCC
ACCCTGACCGCCGACAAGAGCTCCTCCACCGCGTATATGCAGTTG
AGCTCCCTGACCTCCGAGGACTCCGCCGACTACTACTGCGCACGG
TCCAACTACTATGGAAGCTCGTACTGGTTCTTCGATGTCTGGGGG
GCCGGCACCACTGTGACCGTCAGCTCCGGGGGCGGAGGATCCGGT
GGAGGCGGAAGCGGGGGTGGAGGATCCGACATTGTGCTGACTCAG
TCCCCGGCAATCCTGTCGGCCTCACCGGGCGAAAAGGTCACGATG
ACTTGTAGAGCGTCGTCCAGCGTGAACTACATGGATTGGTACCAA
AAGAAGCCTGGATCGTCACCCAAGCCTTGGATCTACGCTACATCT
AACCTGGCCTCCGGCGTGCCAGCGCGGTTCAGCGGGTCCGGCTCG
GGCACCTCATACTCGCTGACCATCTCCCGCGTGGAGGCTGAGGAC
GCCGCGACCTACTACTGCCAGCAGTGGTCCTTCAACCCGCCGACT
TTTGGAGGCGGTACTAAGCTGGAGATCAAAGGAGGCGGCGGCAGC
GGCGGGGGAGGGTCCGGAGGGGGTGGTTCTGGTGGAGGAGGATCG
GGAGGCGGTGGCAGCGACATTCAGATGACTCAGACCACCTCCTCC
CTGTCCGCCTCCCTGGGCGACCGCGTGACCATCTCATGCCGCGCC
AGCCAGGACATCTCGAAGTACCTCAACTGGTACCAGCAGAAGCCC
GACGGAACCGTGAAGCTCCTGATCTACCACACCTCCCGGCTGCAC
AGCGGAGTGCCGTCTAGATTCTCGGGTTCGGGGTCGGGAACTGAC
TACTCCCTTACTATTTCCAACCTGGAGCAGGAGGATATTGCCACC
TACTTCTGCCAACAAGGAAACACCCTGCCGTACACTTTTGGCGGG
GGAACCAAGCTGGAAATCACTGGCAGCACATCCGGTTCCGGGAAG
CCCGGCTCCGGAGAGGGCAGCACCAAGGGGGAAGTCAAGCTGCAG
GAATCAGGACCTGGCCTGGTGGCCCCGAGCCAGTCACTGTCCGTG
ACTTGTACTGTGTCCGGAGTGTCGCTCCCGGATTACGGAGTGTCC
TGGATCAGGCAGCCACCTCGGAAAGGATTGGAATGGCTCGGAGTC
ATCTGGGGTTCCGAAACCACCTATTACAACTCGGCACTGAAATCC
AGGCTCACCATTATCAAGGATAACTCCAAGTCACAAGTGTTCCTG
AAGATGAATAGCCTGCAGACTGACGACACGGCGATCTACTATTGC
GCCAAGCACTACTACTACGGCGGATCCTACGCTATGGACTACTGG
GGCCAGGGGACCAGCGTGACCGTGTCATCCGCGGCCGCAACTACC
ACCCCTGCCCCTCGGCCGCCGACTCCGGCCCCAACCATCGCAAGC
CAACCCCTCTCCTTGCGCCCCGAAGCTTGCCGCCCGGCCGCGGGT
GGAGCCGTGCATACCCGGGGGCTGGACTTTGCCTGCGATATCTAC
ATTTGGGCCCCGCTGGCCGGCACTTGCGGCGTGCTCCTGCTGTCG
CTGGTCATCACCCTTTACTGCAAGAGGGGCCGGAAGAAGCTGCTT
TACATCTTCAAGCAGCCGTTCATGCGGCCCGTGCAGACGACTCAG
GAAGAGGACGGATGCTCGTGCAGATTCCCTGAGGAGGAAGAGGGG
GGATGCGAACTGCGCGTCAAGTTCTCACGGTCCGCCGACGCCCCC
GCATATCAACAGGGCCAGAATCAGCTCTACAACGAGCTGAACCTG
GGAAGGAGAGAGGAGTACGACGTGCTGGACAAGCGACGCGGACGC
GACCCGGAGATGGGGGGGAAACCACGGCGGAAAAACCCTCAGGAA
GGACTGTACAACGAACTCCAGAAAGACAAGATGGCGGAAGCCTAC
TCAGAAATCGGGATGAAGGGAGAGCGGAGGAGGGGAAAGGGTCAC
GACGGGCTGTACCAGGGACTGAGCACCGCCACTAAGGATACCTAC
GATGCCTTGCATATGCAAGCACTCCCACCCCGGTAG
amino acid sequence of CAR LTG1497
(mCD20_CD19 CD8 BBz)
SEQ ID NO: 20
MLLLVTSLLLCELPHPAFLLIPEVQLQQSGAELVKPGASVKMSCK
ASGYTFTSYNMHWVKQTPGQGLEWIGAIYPGNGDTSYNQKFKGKA
TLTADKSSSTAYMQLSSLTSEDSADYYCARSNYYGSSYWFFDVWG
AGTTVTVSSGGGGSGGGGSGGGGSDIVLTQSPAILSASPGEKVTM
TCRASSSVNYMDWYQKKPGSSPKPWIYATSNLASGVPARFSGSGS
GTSYSLTISRVEAEDAATYYCQQWSFNPPTFGGGTKLEIKGGGGS
GGGGSGGGGSGGGGSGGGGSDIQMTQTTSSLSASLGDRVTISCRA
SQDISKYLNWYQQKPDGTVKLLIYHTSRLHSGVPSRFSGSGSGTD
YSLTISNLEQEDIATYFCQQGNTLPYTFGGGTKLEITGSTSGSGK
PGSGEGSTKGEVKLQESGPGLVAPSQSLSVTCTVSGVSLPDYGVS
WIRQPPRKGLEWLGVIWGSETTYYNSALKSRLTIIKDNSKSQVFL
KMNSLQTDDTAIYYCAKHYYYGGSYAMDYWGQGTSVTVSSAAATT
TPAPRPPTPAPTIASQPLSLRPEACRPAAGGAVHTRGLDFACDIY
IWAPLAGTCGVLLLSLVITLYCKRGRKKLLYIFKQPFMRPVQTTQ
EEDGCSCRFPEEEEGGCELRVKFSRSADAPAYQQGQNQLYNELNL
GRREEYDVLDKRRGRDPEMGGKPRRKNPQEGLYNELQKDKMAEAY
SEIGMKGERRRGKGHDGLYQGLSTATKDTYDALHMQALPPR
nucleotide sequence of CD28 co-stimulatory
domain
SEQ ID NO: 21
AGGAGTAAACGCAGCCGCCTGCTGCATTCAGACTACATGAACATG
ACCCCACGGCGGCCCGGCCCAACGCGCAAACACTACCAACCTTAC
GCCCCACCGCGAGACTTTGCCGCCTACAGATCC
amino acid sequence of CD28 co-stimulatory
domain
SEQ ID NO: 22
RSKRSRLLHSDYMNMTPRRPGPTRKHYQPY APPRDFAAYRS
nucleotide sequence of 4-1BB co-stimulatory
domain
SEQ ID NO: 23
AAGCGCGGACGGAAGAAACTCTTGTACATCTTCAAGCAGCCGTTC
ATGCGCCCTGTGCAAACCACCCAAGAAGAGGACGGGTGCTCCTGC
CGGTTCCCGGAAGAGGAAGAGGGCGGCTGCGAACTG
amino acid sequence of 4-1BB co-stimulatory
domain
SEQ ID NO: 24
KRGRKKLLYIFKQPFMRPVQTTQEEDGCSCRFPEEEEGGCEL
nucleotide sequence of tandem CD28_4-1BB
co-stimulatory domain
SEQ ID NO: 25
AGGAGTAAACGCAGCCGCCTGCTGCATTCAGACTACATGAACATG
ACCCCACGGCGGCCCGGCCCAACGCGCAAACACTACCAACCTTAC
GCCCCACCGCGAGACTTTGCCGCCTACAGATCCAAGCGCGGACGG
AAGAAACTCTTGTACATCTTCAAGCAGCCGTTCATGCGCCCTGTG
CAAACCACCCAAGAAGAGGACGGGTGCTCCTGCCGGTTCCCGGAA
GAGGAAGAGGGCGGCTGCGAACTG
amino acid sequence of tandem CD28_4-1BB
co-stimulatory domain
SEQ ID NO: 26
RSKRSRLLHSDYMNMTPRRPGPTRKHYQPYAPPRDFAAYRSKRGR
KKLLYIFKQPFMRPVQTTQEEDGCSCRFPEEEEGGCEL
nucleotide sequence of OX40 co-stimulatory
domain
SEQ ID NO: 27
GTGGCGGCAATTCTCGGCCTGGGACTTGTCCTTGGTCTGCTTGGT
CCGCTCGCAATACTTCTGGCCTTGTACCTGCTCCGCAGAGACCAA
AGACTTCCGCCCGACGCCCACAAGCCCCCAGGAGGAGGTTCCTTC
AGAACGCCTATACAAGAAGAACAAGCAGATGCCCACTCTACCCTG
GCTAAAATC
amino acid sequence of OX40 co-stimulatory
domain
SEQ ID NO: 28
VAAILGLGLVLGLLGPLAILLALYLLRRDQRLPPDAHKPPGGGSF
RTPIQEEQADAHSTLAKI
nucleotide sequence of ICOS co-stimulatory
domain
SEQ ID NO: 29
TGGCTGACAAAAAAGAAGTATTCATCTAGTGTACATGATCCGAAC
GGTGAATACATGTTCATGCGCGCGGTGAACACGGCCAAGAAGAGC
AGACTGACCGACGTAACCCTT
amino acid sequence of ICOS co-stimulatory
domain
SEQ ID NO: 30
WLTKKKYSSSVHDPNGEYMFMRAVNTAKKSRLTDVTL
nucleotide sequence of CD28 hinge domain
SEQ ID NO: 31
ATCGAAGTGATGTATCCACCTCCGTACCTCGATAACGAGAAATCA
AATGGAACGATCATTCATGTGAAAGGGAAACATCTGTGCCCAAGC
CCATTGTTCCCAGGTCCGTCAAAACCA
amino acid sequence of CD28 hinge domain
SEQ ID NO: 32
IEVMYPPPYLDNEKSNGTIIHVKGKHLCPSPLFPGPSKP
nucleotide sequence of OX40 transmembrane
domain
SEQ ID NO: 33
GTGGCGGCAATTCTCGGCCTGGGACTTGTCCTTGGTCTGCTTGGT
CCGCTCGCAATACTTCTG
amino acid sequence of OX40 transmembrane
domain
SEQ ID NO: 34
VAAILGLGLVLGLLGPLAILL
nucleotide sequence of DNA CD8 transmembrane
domain
SEQ ID NO: 35
atttgggccccgctggccggcacttgcggcgtgctcctgctgtcg
ctggtcatcaccctttactgc
amino acid sequence of CD8 transmembrane
domain
SEQ ID NO: 36
Ile Trp Ala Pro Leu Ala Gly Thr Cys Gly
Val Leu Leu Leu Ser Leu Val Ile Thr Leu
Tyr Cys
nucleotide sequence of DNA CD8 hinge domain
SEQ ID NO: 37
actaccacccctgcccctcggccgccgactccggccccaaccatc
gcaagccaacccctctccttgcgccccgaagcttgccgcccggcc
gcgggtggagccgtgcatacccgggggctggactttgcctgcgat
atctac
amino acid sequence of CD8 hinge domain
SEQ ID NO: 38
Thr Thr Thr Pro Ala Pro Arg Pro Pro
Thr Pro Ala Pro Thr Ile Ala Ser GIn
Pro Leu Ser Leu Arg Pro Glu Ala Cys
Arg Pro Ala Ala Gly Gly Ala Val His
Thr Arg Gly Leu Asp Phe Ala Cys Asp
Ile Tyr
amino acid sequence of amino acid numbers
137 to 206 hinge and transmembrane
region of CD8.alpha.
(NCBI RefSeq: NP.sub.--001759.3)
SEQ ID NO: 39
Thr Thr Thr Pro Ala Pro Arg Pro Pro
Thr Pro Ala Pro Thr Ile Ala Ser Gln
Pro Leu Ser Leu Arg Pro Glu Ala Cys
Arg Pro Ala Ala Gly Gly Ala Val His
Thr Arg Gly Leu Asp Phe Ala Cys Asp
Ile Tyr Ile Trp Ala Pro Leu Ala Gly
Thr Cys Gly Val Leu Leu Leu Ser Leu
Val Ile Thr Leu Tyr Cys
nucleotide sequence of DNA signaling
domain of 4-1BB
SEQ ID NO: 40
aagaggggccggaagaagctgctttacatcttcaagcagccgttc
atgcggcccgtgcagacgactcaggaagaggacggatgctcgtgc
agattccctgaggaggaagaggggggatgcgaactg
amino acid sequence of signaling domain
of 4-1BB
SEQ ID NO: 41
Lys Arg Gly Arg Lys Lys Leu Leu Tyr
Ile Phe Lys Gln Pro Phe Met Arg Pro
Val Gln Thr Thr Gln Glu Glu Asp Gly
Cys Ser Cys Arg Phe Pro Glu Glu Glu
Glu Gly Gly Cys Glu Leu
nucleotide sequence of DNA signaling
domain of CD3-zeta
SEQ ID NO: 42
CGCGTGAAGTTTTCCCGGTCCGCCGACGCTCCGGCGTACCAGCAG
GGGCAAAACCAGCTGTACAACGAACTTAACCTCGGTCGCCGGGAA
GAATATGACGTGCTGGACAAGCGGCGGGGAAGAGATCCCGAGATG
GGTGGAAAGCCGCGGCGGAAGAACCCTCAGGAGGGCTTGTACAAC
GAGCTGCAAAAGGACAAAATGGCCGAAGCCTACTCCGAGATTGGC
ATGAAGGGAGAGCGCAGACGCGGGAAGGGACACGATGGACTGTAC
CAGGGACTGTCAACCGCGACTAAGGACACTTACGACGCCCTGCAC
ATGCAGGCCCTGCCCCCGCGC
amino acid sequence of CD3zeta
SEQ ID NO: 43
RVKFSRSADAPAYQQGQNQLYNELNLGRREEYDVLDKRRGRDPEM
GGKPRRKNPQEGLYNELQKDKMAEAYSEIGMKGERRRGKGHDGLY
QGLSTATKDTYDALHMQALPPR
alternative nucleotide sequence of CD3zeta 1
SEQ ID NO: 44
AGGGTGAAGTTTAGCCGCTCAGCCGATGCACCGGCCTACCAGCAG
GGACAGAACCAGCTCTACAACGAGCTCAACCTGGGTCGGCGGGAA
GAATATGACGTGCTGGACAAACGGCGCGGCAGAGATCCGGAGATG
GGGGGAAAGCCGAGGAGGAAGAACCCTCAAGAGGGCCTGTACAAC
GAACTGCAGAAGGACAAGATGGCGGAAGCCTACTCCGAGATCGGC
ATGAAGGGAGAACGCCGGAGAGGGAAGGGTCATGACGGACTGTAC
CAGGGCCTGTCAACTGCCACTAAGGACACTTACGATGCGCTCCAT
ATGCAAGCTTTGCCCCCGCGG
alternative nucleotide sequence of CD3zeta 2
SEQ ID NO: 45
AGAGTCAAATTTTCCAGGTCCGCAGATGCCCCCGCGTACCAGCAA
GGCCAGAACCAACTTTACAACGAACTGAACCTGGGTCGCCGGGAG
GAATATGATGTGCTGGATAAACGAAGGGGGAGGGACCCTGAGATG
GGAGGGAAACCTCGCAGGAAAAACCCGCAGGAAGGTTTGTACAAC
GAGTTGCAGAAGGATAAGATGGCTGAGGCTTACTCTGAAATAGGG
ATGAAGGGAGAGAGACGGAGAGGAAAAGGCCATGATGGCCTTTAC
CAGGGCTTGAGCACAGCAACAAAGGATACTTACGACGCTCTTCAC
ATGCAAGCTCTGCCACCACGG
nucleotide sequence of CD28 transmembrane
domain
SEQ ID NO: 46
TTCTGGGTGCTTGTCGTTGTTGGGGGTGTACTCGCATGTTATTCT
TTGCTGGTGACTGTGGCGTTTATCATCTTCTGGGTA
amino acid sequence of CD28 transmembrane
domain
SEQ ID NO: 47
FWVLVVVGGVLACYSLLVTVAFIIFWV
nucleotide sequence of CD20/CD19-
reactive ScFv binding domain
SEQ ID NO: 48
GAAATTGTGTTGACTCAGTCACCGGCAACTCTTAGTCTCTCCCCT
GGAGAGAGAGCAACACTGAGCTGCAGAGCATCTCAAAGTGTTTCC
AGCTATCTGGCCTGGTACCAGCAAAAGCCAGGCCAGGCACCACGA
CTTCTGATTTACGACGCAAGTAACAGGGCGACTGGCATCCCCGCC
CGCTTTTCCGGATCAGGCAGTGGCACAGACTTTACTTTGACGATC
AGTTCTTTGGAGCCAGAGGACTTTGCTGTTTACTACTGTCAGCAG
AGATCCAATTGGCCGATTACCTTCGGCCAAGGTACCAGGCTGGAG
ATAAAGGGTGGCGGCGGCTCTGGTGGAGGTGGTTCCGGTGGGGGA
GGTAGCGAAGTGCAATTGGTGGAAAGCGGTGGTGGCTTGGTTCAA
CCTGGGCGGTCCTTGAGATTGTCATGCGCCGCATCCGGCTTCACC
TTCAACGACTATGCAATGCACTGGGTGCGGCAAGCTCCAGGCAAG
GGCCTTGAGTGGGTCAGCACTATTAGCTGGAACTCCGGGAGTATC
GGATACGCTGATTCAGTCAAAGGTCGATTTACCATTTCCCGCGAT
AATGCCAAAAAAAGCTTGTATCTCCAAATGAACTCACTCAGGGCT
GAAGACACCGCGCTGTATTACTGTGCTAAAGACATCCAATATGGC
AATTACTATTACGGGATGGATGTATGGGGGCAAGGGACAACGGTC
ACTGTCTCATCCGGTGGAGGAGGTTCAGGCGGAGGAGGCTCAGGC
GGAGGCGGTAGCGGCGGAGGAGGAAGCGGAGGTGGCGGATCAGAG
GTCCAGCTGGTACAGTCTGGAGCTGAGGTGAAGAAGCCTGGGGCC
TCAGTGAAGGTCTCCTGCAAGGCTTCTGGATACACCTTCACCAGC
TACTATATGCATTGGGTGCGACAGGCCCCTGGACAAGGGCTTGAG
TGGATGGGATTAATCAACCCTAGTGGTGGTAGCACAAGCTACGCA
CAGAAGTTCCAGGGCAGAGTCACCATGACCAGGGACACGTCCACG
AGCACAGTCTACATGGAGCTGAGCAGCCTGAGATCTGAGGACACG
GCCGTGTACTATTGTGCGAGATCGGATCGGGGAATTACCGCCACG
GACGCTTTTGATATCTGGGGCCAAGGGACAATGGTCACCGTCTCT
TCAGGCGGTGGAGGCTCAGGTGGAGGAGGTAGCGGAGGCGGTGGT
TCCCAGTCTGTGCTGACTCAGCCACCCTCGGTGTCAGTGGCCCCA
GGGCGGATGGCCAAGATTACCTGTGGGGGAAGTGACATTGGAAAT
AAAAATGTCCACTGGTATCAGCAGAAGCCTGGCCAGGCTCCTGTC
CTGGTTGTCTATGATGATTACGACCGGCCCTCAGGGATCCCTGAG
CGATTTTCAGGCTCCAACTCTGGGGACGCGGCCACCCTGACGATC
AGCACGGTCGAAGTCGGGGATGAGGCCGACTATTTCTGTCAGGTG
TGGGACGGTAGTGGTGATCCTTATTGGATGTTCGGCGGAGGGACC
CAGCTCACCGTTTTAGGT
amino acid sequence of CD20/CD19-
reactive ScFv binding domain
SEQ ID NO: 49
EIVLTQSPATLSLSPGERATLSCRASQSVSSYLAWYQQKPGQAPR
LLIYDASNRATGIPARFSGSGSGTDFTLTISSLEPEDFAVYYCQQ
RSNWPITFGQGTRLEIKGGGGSGGGGSGGGGSEVQLVESGGGLVQ
PGRSLRLSCAASGFTENDYAMHWVRQAPGKGLEWVSTISWNSGSI
GYADSVKGRFTISRDNAKKSLYLQMNSLRAEDTALYYCAKDIQYG
NYYYGMDVWGQGTTVTVSSGGGGSGGGGSGGGGSGGGGSGGGGSE
VQLVQSGAEVKKPGASVKVSCKASGYTFTSYYMHWVRQAPGQGLE
WMGLINPSGGSTSYAQKFQGRVTMTRDTSTSTVYMELSSLRSEDT
AVYYCARSDRGITATDAFDIWGQGTMVTVSSGGGGSGGGGSGGGG
SQSVLTQPPSVSVAPGRMAKITCGGSDIGNKNVHWYQQKPGQAPV
LVVYDDYDRPSGIPERFSGSNSGDAATLTISTVEVGDEADYFCQV
WDGSGDPYWMFGGGTQLTVLG

1. An isolated nucleic acid molecule encoding a fully human CD20/CD19 tandem chimeric antigen receptor (CAR) comprising at least one extracellular antigen binding domain comprising a fully human CD20/CD19 antigen binding domain, at least one transmembrane domain, and at least one intracellular signaling domain, wherein the fully human CD20/CD19 tandem chimeric antigen receptor (CAR) is encoded by a nucleotide sequence comprising SEQ ID NO. 5, 7, 9, 13, 15, 17, or 19.

2.-13. (canceled)

14. A chimeric antigen receptor (CAR) encoded by the isolated nucleic acid molecule of claim 1.

15.-23. (canceled)

24. A vector comprising a nucleic acid molecule of claim 1.

25. The vector of claim 24, wherein the vector is selected from the group consisting of a DNA vector, an RNA vector, a plasmid vector, a cosmid vector, a herpes virus vector, a measles virus vector, a lentivirus vector, an adenoviral vector, or a retrovirus vector, or a combination thereof.

26. The vector of claim 24, further comprising a promoter.

27. The vector of claim 26, wherein the promoter is an inducible promoter, a constitutive promoter, a tissue specific promoter, a suicide promoter or any combination thereof.

28. A cell comprising the vector of claim 24.

29. The cell of claim 28, wherein the cell is a T cell.

30. The cell of claim 28, wherein the T cell is a CD8+ T cell or a CD4+ T cell.

31. The cell of claim 28, wherein the cell is a human cell.

32. A method of making a cell comprising transducing a T cell with a vector of claim 24.

33. A method of generating a population of RNA-engineered cells comprising introducing an in vitro transcribed RNA or synthetic RNA into a cell, where the RNA comprises a nucleic acid molecule of claim 1.

34.-35. (canceled)

36. A pharmaceutical composition comprising an anti-tumor effective amount of a population of human T cells, wherein the T cells comprise a nucleic acid sequence that encodes a chimeric antigen receptor (CAR), wherein the CAR comprises at least one extracellular antigen binding domain comprising a fully human CD20/CD19 antigen binding domain comprising the amino acid sequence of SEQ ID NO. 2, 4, or 49, at least one linker domain, at least one transmembrane domain, at least one intracellular signaling domain, and wherein the T cells are T cells of a human having a cancer.

37. The pharmaceutical composition of claim 36, wherein the at least one transmembrane domain comprises a transmembrane domain of a protein comprising the alpha, beta or zeta chain of the T-cell receptor, CD8, CD28, CD3 epsilon, CD45, CD4, CD5, CD8, CD9, CD16, CD22, CD33, CD37, CD64, CD80, CD86, CD134, CD137, CD154, and TNFRSF19, or any combination thereof.

38. The pharmaceutical composition of claim 36, wherein the T cells are T cells of a human having a hematological cancer.

39.-44. (canceled)

45. A method of treating cancer in a subject in need thereof, the method comprising administering to the subject the pharmaceutical composition of claim 36.

46. The method of claim 45, wherein the at least one transmembrane domain comprises a transmembrane domain of a protein comprising the alpha, beta or zeta chain of the T-cell receptor, CD8, CD28, CD3 epsilon, CD45, CD4, CD5, CD8, CD9, CD16, CD22, CD33, CD37, CD64, CD80, CD86, CD134, CD137 and CD154, or any combination thereof.

47.-48. (canceled)

49. A pharmaceutical composition comprising an anti-allergic, anti-autoimmune, anti-alloimmune, or anti-autoaggressive effective amount of a population of human T cells, wherein the T cells comprise a nucleic acid sequence that encodes a chimeric antigen receptor (CAR), wherein the CAR comprises at least one extracellular antigen binding domain comprising a fully human CD20/CD19 antigen binding domain comprising the amino acid sequence of SEQ ID NO. 2, 4, or 49, at least one linker domain, at least one transmembrane domain, at least one intracellular signaling domain, and wherein the T cells are T cells of a human having a disease.

50. The pharmaceutical composition of claim 49, wherein the disease includes allergic disease including asthma, atopic eczema, rhinitis, skin allergy, Type I diabetes, or any combination thereof.

51. The pharmaceutical composition of claim 49, wherein the disease includes autoimmune diseases, alloimmune diseases, and autoaggressive diseases comprising rheumatoid arthritis, lupus, celiac disease, Sjögren's syndrome, multiple sclerosis, polymyalgia rheumatica, ankylosing spondylitis, type 1 diabetes, alopecia areata, vasculitis, temporal arteritis, post-streptococcal autoimmune disorder, antineuronal antibody-mediated neuropsychiatric disorder, immune-mediated extrapyramidal movement disorder, Sydenham chorea, alloimmune hemolytic disease, pulmonary fibrosis, systemic scleroderma, or fibrotic disease, or any combination thereof.


SEQUENCE LISTING (49 sequences)

SEQ ID NO: 1 - DNA, 732 bp, Synthetic construct
  source 1..732: mol_type = other DNA
gaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatcc

SEQ ID NO: 2 - protein, 244 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSS

SEQ ID NO: 3 - DNA, 741 bp, Synthetic construct
  source 1..741: mol_type = other DNA
gaggtccagctggtacagtctggagctgaggtgaagaagcctggggcctcagtgaaggtctcctgcaaggcttctggatacaccttcaccagctactatatgcattgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtactattgtgcgagatcggatcggggaattaccgccacggacgcttttgatatctggggccaagggacaatggtcaccgtctcttcaggcggtggaggctcaggtggaggaggtagcggaggcggtggttcccagtctgtgctgactcagccaccctcggtgtcagtggccccagggcggatggccaagattacctgtgggggaagtgacattggaaataaaaatgtccactggtatcagcagaagcctggccaggctcctgtcctggttgtctatgatgattacgaccggccctcagggatccctgagcgattttcaggctccaactctggggacgcggccaccctgacgatcagcacggtcgaagtcggggatgaggccgactatttctgtcaggtgtgggacggtagtggtgatccttattggatgttcggcggagggacccagctcaccgttttaggt

SEQ ID NO: 4 - protein, 247 aa, Synthetic construct
EVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLG

SEQ ID NO: 5 - DNA, 2295 bp, Synthetic construct
  source 1..2295: mol_type = other DNA
atgctgctgctggtgaccagcctgcttctgtgcgaactgccgcatccggcgtttctgttgattccggaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccggtggaggaggttcaggcggaggaggctcaggcggaggcggtagcggcggaggaggaagcggaggtggcggatcagaggtccagctggtacagtctggagctgaggtgaagaagcctggggcctcagtgaaggtctcctgcaaggcttctggatacaccttcaccagctactatatgcattgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtactattgtgcgagatcggatcggggaattaccgccacggacgcttttgatatctggggccaagggacaatggtcaccgtctcttcaggcggtggaggctcaggtggaggaggtagcggaggcggtggttcccagtctgtgctgactcagccaccctcggtgtcagtggccccagggcggatggccaagattacctgtgggggaagtgacattggaaataaaaatgtccactggtatcagcagaagcctggccaggctcctgtcctggttgtctatgatgattacgaccggccctcagggatccctgagcgattttcaggctccaactctggggacgcggccaccctgacgatcagcacggtcgaagtcggggatgaggccgactatttctgtcaggtgtgggacggtagtggtgatccttattggatgttcggcggagggacccagctcaccgttttaggtgcggccgcaacgaccactcctgcaccccgccctccgactccggccccaaccattgccagccagcccctgtccctgcggccggaagcctgcagaccggctgccggcggagccgtccatacccggggactggatttcgcctgcgatatctatatctgggcaccactcgccggaacctgtggagtgctgctgctgtcccttgtgatcaccctgtactgcaagcgcggacggaagaaactcttgtacatcttcaagcagccgttcatgcgccctgtgcaaaccacccaagaagaggacgggtgctcctgccggttcccggaagaggaagagggcggctgcgaactgcgcgtgaagttttcccggtccgccgacgctccggcgtaccagcaggggcaaaaccagctgtacaacgaacttaacctcggtcgccgggaagaatatgacgtgctggacaagcggcggggaagagatcccgagatgggtggaaagccgcggcggaagaaccctcaggagggcttgtacaacgagctgcaaaaggacaaaatggccgaagcctactccgagattggcatgaagggagagcgcagacgcgggaagggacacgatggactgtaccagggactgtcaaccgcgactaaggacacttacgacgccctgcacatgcaggccctgcccccgcgctaa

SEQ ID NO: 6 - protein, 764 aa, Synthetic construct
MLLLVTSLLLCELPHPAFLLIPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSGGGGSGGGGSEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 7 - DNA, 2409 bp, Synthetic construct
  source 1..2409: mol_type = other DNA
atgctgctgctggtgaccagcctgcttctgtgcgaactgccgcatccggcgtttctgttgattccggaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccggtggaggaggttcaggcggaggaggctcaggcggaggcggtagcggcggaggaggaagcggaggtggcggatcagaggtccagctggtacagtctggagctgaggtgaagaagcctggggcctcagtgaaggtctcctgcaaggcttctggatacaccttcaccagctactatatgcattgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtactattgtgcgagatcggatcggggaattaccgccacggacgcttttgatatctggggccaagggacaatggtcaccgtctcttcaggcggtggaggctcaggtggaggaggtagcggaggcggtggttcccagtctgtgctgactcagccaccctcggtgtcagtggccccagggcggatggccaagattacctgtgggggaagtgacattggaaataaaaatgtccactggtatcagcagaagcctggccaggctcctgtcctggttgtctatgatgattacgaccggccctcagggatccctgagcgattttcaggctccaactctggggacgcggccaccctgacgatcagcacggtcgaagtcggggatgaggccgactatttctgtcaggtgtgggacggtagtggtgatccttattggatgttcggcggagggacccagctcaccgttttaggtgcggccgcaatcgaagtgatgtatccacctccgtacctcgataacgagaaatcaaatggaacgatcattcatgtgaaagggaaacatctgtgcccaagcccattgttcccaggtccgtcaaaaccattctgggtgcttgtcgttgttgggggtgtactcgcatgttattctttgctggtgactgtggcgtttatcatcttctgggtaaggagtaaacgcagccgcctgctgcattcagactacatgaacatgaccccacggcggcccggcccaacgcgcaaacactaccaaccttacgccccaccgcgagactttgccgcctacagatccaagcgcggacggaagaaactcttgtacatcttcaagcagccgttcatgcgccctgtgcaaaccacccaagaagaggacgggtgctcctgccggttcccggaagaggaagagggcggctgcgaactgcgcgtgaagttttcccggtccgccgacgctccggcgtaccagcaggggcaaaaccagctgtacaacgaacttaacctcggtcgccgggaagaatatgacgtgctggacaagcggcggggaagagatcccgagatgggtggaaagccgcggcggaagaaccctcaggagggcttgtacaacgagctgcaaaaggacaaaatggccgaagcctactccgagattggcatgaagggagagcgcagacgcgggaagggacacgatggactgtaccagggactgtcaaccgcgactaaggacacttacgacgccctgcacatgcaggccctgcccccgcgctaa

SEQ ID NO: 8 - protein, 802 aa, Synthetic construct
MLLLVTSLLLCELPHPAFLLIPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSGGGGSGGGGSEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGAAAIEVMYPPPYLDNEKSNGTIIHVKGKHLCPSPLFPGPSKPFWVLVVVGGVLACYSLLVTVAFIIFWVRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 9 - DNA, 2418 bp, Synthetic construct
  source 1..2418: mol_type = other DNA
atgctgctgctggtgaccagcctgcttctgtgcgaactgccgcatccggcgtttctgttgattccggaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccggtggaggaggttcaggcggaggaggctcaggcggaggcggtagcggcggaggaggaagcggaggtggcggatcagaggtccagctggtacagtctggagctgaggtgaagaagcctggggcctcagtgaaggtctcctgcaaggcttctggatacaccttcaccagctactatatgcattgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtactattgtgcgagatcggatcggggaattaccgccacggacgcttttgatatctggggccaagggacaatggtcaccgtctcttcaggcggtggaggctcaggtggaggaggtagcggaggcggtggttcccagtctgtgctgactcagccaccctcggtgtcagtggccccagggcggatggccaagattacctgtgggggaagtgacattggaaataaaaatgtccactggtatcagcagaagcctggccaggctcctgtcctggttgtctatgatgattacgaccggccctcagggatccctgagcgattttcaggctccaactctggggacgcggccaccctgacgatcagcacggtcgaagtcggggatgaggccgactatttctgtcaggtgtgggacggtagtggtgatccttattggatgttcggcggagggacccagctcaccgttttaggtgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgcaggagtaaacgcagccgcctgctgcattcagactacatgaacatgaccccacggcggcccggcccaacgcgcaaacactaccaaccttacgccccaccgcgagactttgccgcctacagatccaagcgcggacggaagaaactcttgtacatcttcaagcagccgttcatgcgccctgtgcaaaccacccaagaagaggacgggtgctcctgccggttcccggaagaggaagagggcggctgcgaactgcgcgtgaagttttcccggtccgccgacgctccggcgtaccagcaggggcaaaaccagctgtacaacgaacttaacctcggtcgccgggaagaatatgacgtgctggacaagcggcggggaagagatcccgagatgggtggaaagccgcggcggaagaaccctcaggagggcttgtacaacgagctgcaaaaggacaaaatggccgaagcctactccgagattggcatgaagggagagcgcagacgcgggaagggacacgatggactgtaccagggactgtcaaccgcgactaaggacacttacgacgccctgcacatgcaggccctgcccccgcgctaa

SEQ ID NO: 10 - protein, 805 aa, Synthetic construct
MLLLVTSLLLCELPHPAFLLIPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSGGGGSGGGGSEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 11 - DNA, 66 bp, Synthetic construct
  source 1..66: mol_type = other DNA
atgctgctgctggtgaccagcctgctgctgtgcgaactgccgcatccggcgtttctgctgattccg

SEQ ID NO: 12 - protein, 22 aa, Synthetic construct
MLLLVTSLLLCELPHPAFLLIP

SEQ ID NO: 13 - DNA, 3054 bp, Synthetic construct
  source 1..3054: mol_type = other DNA
atgctgctgctggtgaccagcctgcttctgtgcgaactgccgcatccggcgtttctgttgattccggaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccgcggccgcgactaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgccggtcgaagaggtccagactcttgcactccgactacatgaacatgactcctagaaggcccggacccactagaaagcactaccagccgtacgcccctcctcgggatttcgccgcataccggtccagagtgaagttcagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatggctctgcctgttacggcactgctccttccgcttgcattgttgttgcacgcagcgcggcccgaggtccagctggtacagtctggagctgaggtgaagaagcctggggcctcagtgaaggtctcctgcaaggcttctggatacaccttcaccagctactatatgcactgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtattactgtgcgagatcggatcggggaattaccgccacggacgcttttgatatctggggccaagggacaatggtcaccgtctcttcaggcggaggaggctccgggggaggaggttccgggggcgggggttcccagtctgtgctgactcagccaccctcggtgtcagtggccccagggcggatggccaagattacctgtgggggaagtgacattggaaataaaaatgtccactggtatcagcagaagccaggccaggcccctgtcctggttgtctatgatgattacgaccggccctcagggatccctgagcgattctctggctccaactctggggacgcggccaccctgacgatcagcacggtcgaagtcggggatgaggccgactatttctgtcaggtgtgggacggtagtggtgatccttattggatgttcggcggagggacccagctcaccgttttaggtgctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgcaagaggggccggaagaagctgctttacatcttcaagcagccgttcatgcggcccgtgcagacgactcaggaagaggacggatgctcgtgcagattccctgaggaggaagaggggggatgcgaactgagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacggtag

SEQ ID NO: 14 - protein, 1017 aa, Synthetic construct
MLLLVTSLLLCELPHPAFLLIPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSAAATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGASATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 15 - DNA, 3054 bp, Synthetic construct
  source 1..3054: mol_type = other DNA
atgctgctgctggtgaccagcctgcttctgtgcgaactgccgcatccggcgtttctgttgattccggaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccgcggccgcaacgaccactcctgcaccacggccacctaccccagcccccaccattgcaagccagccactttcactgcgccccgaagcgtgtagaccagctgctggaggagccgtgcatacccgagggctggacttcgcctgtgacatctacatctgggccccattggctggaacttgcggcgtgctgctcttgtctctggtcattaccctgtactgcaagcgcggacggaagaaactcttgtacatcttcaagcagccgttcatgcgccctgtgcaaaccacccaagaagaggacgggtgctcctgccggttcccggaagaggaagagggcggctgcgaactgagagtgaagtttagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatggctctgcctgttacggcactgctccttccgcttgcattgttgttgcacgcagcgcggcccgaggtccagctggtacagtctggagctgaggtgaagaagcctggggcctcagtgaaggtctcctgcaaggcttctggatacaccttcaccagctactatatgcactgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtattactgtgcgagatcggatcggggaattaccgccacggacgcttttgatatctggggccaagggacaatggtcaccgtctcttcaggcggaggaggctccgggggaggaggttccgggggcgggggttcccagtctgtgctgactcagccaccctcggtgtcagtggccccagggcggatggccaagattacctgtgggggaagtgacattggaaataaaaatgtccactggtatcagcagaagccaggccaggcccctgtcctggttgtctatgatgattacgaccggccctcagggatccctgagcgattctctggctccaactctggggacgcggccaccctgacgatcagcacggtcgaagtcggggatgaggccgactatttctgtcaggtgtgggacggtagtggtgatccttattggatgttcggcggagggacccagctcaccgttttaggtgctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgccggtcgaagaggtccagactcttgcactccgactacatgaacatgactcctagaaggcccggacccactagaaagcactaccagccgtacgcccctcctcgggatttcgccgcataccggtccagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttaagcacagcaacaaaggatacttacgacgctcttcacatgcaagctctgccaccacggtag

SEQ ID NO: 16 - protein, 1017 aa, Synthetic construct
MLLLVTSLLLCELPHPAFLLIPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGASATTTPAPRPPTPAPTIASQPLSLRPEACRPAAGGAVHTRGLDFACDIYIWAPLAGTCGVLLLSLVITLYCRSKRSRLLHSDYMNMTPRRPGPTRKHYQPYAPPRDFAAYRSRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 17 - DNA, 3033 bp, Synthetic construct
  source 1..3033: mol_type = other DNA
atgctgctgctggtgaccagcctgcttctgtgcgaactgccgcatccggcgtttctgttgattccggaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccgcggccgcaacgaccactccagcaccgagaccgccaacccccgcgcctaccatcgcaagtcaaccactttctctcaggcctgaagcgtgccgacctgcagctggtggggcagtacataccaggggtttggacttcgcatgtgacgtggcggcaattctcggcctgggacttgtccttggtctgcttggtccgctcgcaatacttctggccttgtacctgctccgcagagaccaaagacttccgcccgacgcccacaagcccccaggaggaggttccttcagaacgcctatacaagaagaacaagcagatgcccactctaccctggctaaaatcagggtgaagtttagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcggcgcgcgaaacgcggcagcggcgcgaccaactttagcctgctgaaacaggcgggcgatgtggaagaaaacccgggcccgcgagcaaagaggaatattatggctctgcctgttacggcactgctccttccgcttgcattgttgttgcacgcagcgcggcccgaggtccagctggtacagtctggagctgaggtgaagaagcctggggcctcagtgaaggtctcctgcaaggcttctggatacaccttcaccagctactatatgcactgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtattactgtgcgagatcggatcggggaattaccgccacggacgcttttgatatctggggccaagggacaatggtcaccgtctcttcaggcggaggaggctccgggggaggaggttccgggggcgggggttcccagtctgtgctgactcagccaccctcggtgtcagtggccccagggcggatggccaagattacctgtgggggaagtgacattggaaataaaaatgtccactggtatcagcagaagccaggccaggcccctgtcctggttgtctatgatgattacgaccggccctcagggatccctgagcgattctctggctccaactctggggacgcggccaccctgacgatcagcacggtcgaagtcggggatgaggccgactatttctgtcaggtgtgggacggtagtggtgatccttattggatgttcggcggagggacccagctcaccgttttaggtgctagcgcaaccactacgcctgctccgcggcctccaacgcccgcgcccacgatagctagtcagccgttgtctctccgaccagaggcgtgtagaccggccgctggcggagccgtacatactcgcggactcgacttcgcttgcgacatctacatttgggcacccttggctgggacctgtggggtgctgttgctgtccttggttattacgttgtactgctggctgacaaaaaagaagtattcatctagtgtacatgatccgaacggtgaatacatgttcatgcgcgcggtgaacacggccaagaagagcagactgaccgacgtaacccttagagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacggtag

SEQ ID NO: 18 - protein, 1010 aa, Synthetic construct
MLLLVTSLLLCELPHPAFLLIPEIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSAAATTTPAPRPPTPAPTIASQPLSLRPEACRPAAGGAVHTRGLDFACDVAAILGLGLVLGLLGPLAILLALYLLRRDQRLPPDAHKPPGGGSFRTPIQEEQADAHSTLAKIRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPRRAKRGSGATNFSLLKQAGDVEENPGPRAKRNIMALPVTALLLPLALLLHAARPEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLGASATTTPAPRPPTPAPTIASQPLSLRPEACRPAAGGAVHTRGLDFACDIYIWAPLAGTCGVLLLSLVITLYCWLTKKKYSSSVHDPNGEYMFMRAVNTAKKSRLTDVTLRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 19 - DNA, 2286 bp, Synthetic construct
  source 1..2286: mol_type = other DNA
atgctccttctcgtgacctccctgcttctctgcgaactgccccatcctgccttcctgctgattcccgaggtgcagttgcaacagtcaggagctgaactggtcaagccaggagccagcgtgaagatgagctgcaaggcctccggttacaccttcacctcctacaacatgcactgggtgaaacagaccccgggacaagggctcgaatggattggcgccatctaccccgggaatggcgatacttcgtacaaccagaagttcaagggaaaggccaccctgaccgccgacaagagctcctccaccgcgtatatgcagttgagctccctgacctccgaggactccgccgactactactgcgcacggtccaactactatggaagctcgtactggttcttcgatgtctggggggccggcaccactgtgaccgtcagctccgggggcggaggatccggtggaggcggaagcgggggtggaggatccgacattgtgctgactcagtccccggcaatcctgtcggcctcaccgggcgaaaaggtcacgatgacttgtagagcgtcgtccagcgtgaactacatggattggtaccaaaagaagcctggatcgtcacccaagccttggatctacgctacatctaacctggcctccggcgtgccagcgcggttcagcgggtccggctcgggcacctcatactcgctgaccatctcccgcgtggaggctgaggacgccgcgacctactactgccagcagtggtccttcaacccgccgacttttggaggcggtactaagctggagatcaaaggaggcggcggcagcggcgggggagggtccggagggggtggttctggtggaggaggatcgggaggcggtggcagcgacattcagatgactcagaccacctcctccctgtccgcctccctgggcgaccgcgtgaccatctcatgccgcgccagccaggacatctcgaagtacctcaactggtaccagcagaagcccgacggaaccgtgaagctcctgatctaccacacctcccggctgcacagcggagtgccgtctagattctcgggttcggggtcgggaactgactactcccttactatttccaacctggagcaggaggatattgccacctacttctgccaacaaggaaacaccctgccgtacacttttggcgggggaaccaagctggaaatcactggcagcacatccggttccgggaagcccggctccggagagggcagcaccaagggggaagtcaagctgcaggaatcaggacctggcctggtggccccgagccagtcactgtccgtgacttgtactgtgtccggagtgtcgctcccggattacggagtgtcctggatcaggcagccacctcggaaaggattggaatggctcggagtcatctggggttccgaaaccacctattacaactcggcactgaaatccaggctcaccattatcaaggataactccaagtcacaagtgttcctgaagatgaatagcctgcagactgacgacacggcgatctactattgcgccaagcactactactacggcggatcctacgctatggactactggggccaggggaccagcgtgaccgtgtcatccgcggccgcaactaccacccctgcccctcggccgccgactccggccccaaccatcgcaagccaacccctctccttgcgccccgaagcttgccgcccggccgcgggtggagccgtgcatacccgggggctggactttgcctgcgatatctacatttgggccccgctggccggcacttgcggcgtgctcctgctgtcgctggtcatcaccctttactgcaagaggggccggaagaagctgctttacatcttcaagcagccgttcatgcggcccgtgcagacgactcaggaagaggacggatgctcgtgcagattccctgaggaggaagaggggggatgcgaactgcgcgtcaagttctcacggtccgccgacgcccccgcatatcaacagggccagaatcagctctacaacgagctgaacctgggaaggagagaggagtacgacgtgctggacaagcgacgcggacgcgacccggagatgggggggaaaccacggcggaaaaaccctcaggaaggactgtacaacgaactccagaaagacaagatggcggaagcctactcagaaatcgggatgaagggagagcggaggaggggaaagggtcacgacgggctgtaccagggactgagcaccgccactaaggatacctacgatgccttgcatatgcaagcactcccaccccggtag

SEQ ID NO: 20 - protein, 761 aa, Synthetic construct
MLLLVTSLLLCELPHPAFLLIPEVQLQQSGAELVKPGASVKMSCKASGYTFTSYNMHWVKQTPGQGLEWIGAIYPGNGDTSYNQKFKGKATLTADKSSSTAYMQLSSLTSEDSADYYCARSNYYGSSYWFFDVWGAGTTVTVSSGGGGSGGGGSGGGGSDIVLTQSPAILSASPGEKVTMTCRASSSVNYMDWYQKKPGSSPKPWIYATSNLASGVPARFSGSGSGTSYSLTISRVEAEDAATYYCQQWSFNPPTFGGGTKLEIKGGGGSGGGGSGGGGSGGGGSGGGGSDIQMTQTTSSLSASLGDRVTISCRASQDISKYLNWYQQKPDGTVKLLIYHTSRLHSGVPSRFSGSGSGTDYSLTISNLEQEDIATYFCQQGNTLPYTFGGGTKLEITGSTSGSGKPGSGEGSTKGEVKLQESGPGLVAPSQSLSVTCTVSGVSLPDYGVSWIRQPPRKGLEWLGVIWGSETTYYNSALKSRLTIIKDNSKSQVFLKMNSLQTDDTAIYYCAKHYYYGGSYAMDYWGQGTSVTVSSAAATTTPAPRPPTPAPTIASQPLSLRPEACRPAAGGAVHTRGLDFACDIYIWAPLAGTCGVLLLSLVITLYCKRGRKKLLYIFKQPFMRPVQTTQEEDGCSCRFPEEEEGGCELRVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 21 - DNA, 123 bp, Homo sapiens
  source 1..123: mol_type = genomic DNA
aggagtaaacgcagccgcctgctgcattcagactacatgaacatgaccccacggcggcccggcccaacgcgcaaacactaccaaccttacgccccaccgcgagactttgccgcctacagatcc

SEQ ID NO: 22 - protein, 41 aa, Homo sapiens
RSKRSRLLHSDYMNMTPRRPGPTRKHYQPYAPPRDFAAYRS

SEQ ID NO: 23 - DNA, 126 bp, Homo sapiens
  source 1..126: mol_type = genomic DNA
aagcgcggacggaagaaactcttgtacatcttcaagcagccgttcatgcgccctgtgcaaaccacccaagaagaggacgggtgctcctgccggttcccggaagaggaagagggcggctgcgaactg

SEQ ID NO: 24 - protein, 42 aa, Homo sapiens
KRGRKKLLYIFKQPFMRPVQTTQEEDGCSCRFPEEEEGGCEL

SEQ ID NO: 25 - DNA, 249 bp, Homo sapiens
  source 1..249: mol_type = genomic DNA
aggagtaaacgcagccgcctgctgcattcagactacatgaacatgaccccacggcggcccggcccaacgcgcaaacactaccaaccttacgccccaccgcgagactttgccgcctacagatccaagcgcggacggaagaaactcttgtacatcttcaagcagccgttcatgcgccctgtgcaaaccacccaagaagaggacgggtgctcctgccggttcccggaagaggaagagggcggctgcgaactg

SEQ ID NO: 26 - protein, 83 aa, Homo sapiens
RSKRSRLLHSDYMNMTPRRPGPTRKHYQPYAPPRDFAAYRSKRGRKKLLYIFKQPFMRPVQTTQEEDGCSCRFPEEEEGGCEL

SEQ ID NO: 27 - DNA, 189 bp, Homo sapiens
  source 1..189: mol_type = genomic DNA
gtggcggcaattctcggcctgggacttgtccttggtctgcttggtccgctcgcaatacttctggccttgtacctgctccgcagagaccaaagacttccgcccgacgcccacaagcccccaggaggaggttccttcagaacgcctatacaagaagaacaagcagatgcccactctaccctggctaaaatc

SEQ ID NO: 28 - protein, 63 aa, Homo sapiens
VAAILGLGLVLGLLGPLAILLALYLLRRDQRLPPDAHKPPGGGSFRTPIQEEQADAHSTLAKI

SEQ ID NO: 29 - DNA, 111 bp, Homo sapiens
  source 1..111: mol_type = genomic DNA
tggctgacaaaaaagaagtattcatctagtgtacatgatccgaacggtgaatacatgttcatgcgcgcggtgaacacggccaagaagagcagactgaccgacgtaaccctt

SEQ ID NO: 30 - protein, 37 aa, Homo sapiens
WLTKKKYSSSVHDPNGEYMFMRAVNTAKKSRLTDVTL

SEQ ID NO: 31 - DNA, 117 bp, Homo sapiens
  source 1..117: mol_type = genomic DNA
atcgaagtgatgtatccacctccgtacctcgataacgagaaatcaaatggaacgatcattcatgtgaaagggaaacatctgtgcccaagcccattgttcccaggtccgtcaaaacca

SEQ ID NO: 32 - protein, 39 aa, Homo sapiens
IEVMYPPPYLDNEKSNGTIIHVKGKHLCPSPLFPGPSKP

SEQ ID NO: 33 - DNA, 63 bp, Homo sapiens
  source 1..63: mol_type = genomic DNA
gtggcggcaattctcggcctgggacttgtccttggtctgcttggtccgctcgcaatacttctg

SEQ ID NO: 34 - protein, 21 aa, Homo sapiens
VAAILGLGLVLGLLGPLAILL

SEQ ID NO: 35 - DNA, 66 bp, Homo sapiens
  source 1..66: mol_type = genomic DNA
atttgggccccgctggccggcacttgcggcgtgctcctgctgtcgctggtcatcaccctttactgc

SEQ ID NO: 36 - protein, 22 aa, Homo sapiens
IWAPLAGTCGVLLLSLVITLYC

SEQ ID NO: 37 - DNA, 141 bp, Homo sapiens
  source 1..141: mol_type = genomic DNA
actaccacccctgcccctcggccgccgactccggccccaaccatcgcaagccaacccctctccttgcgccccgaagcttgccgcccggccgcgggtggagccgtgcatacccgggggctggactttgcctgcgatatctac

SEQ ID NO: 38 - protein, 47 aa, Homo sapiens
TTTPAPRPPTPAPTIASQPLSLRPEACRPAAGGAVHTRGLDFACDIY

SEQ ID NO: 39 - protein, 69 aa, Homo sapiens
TTTPAPRPPTPAPTIASQPLSLRPEACRPAAGGAVHTRGLDFACDIYIWAPLAGTCGVLLLSLVITLYC

SEQ ID NO: 40 - DNA, 126 bp, Homo sapiens
  source 1..126: mol_type = genomic DNA
aagaggggccggaagaagctgctttacatcttcaagcagccgttcatgcggcccgtgcagacgactcaggaagaggacggatgctcgtgcagattccctgaggaggaagaggggggatgcgaactg

SEQ ID NO: 41 - protein, 42 aa, Homo sapiens; identical to SEQ ID NO: 24

SEQ ID NO: 42 - DNA, 336 bp, Homo sapiens
  source 1..336: mol_type = genomic DNA
cgcgtgaagttttcccggtccgccgacgctccggcgtaccagcaggggcaaaaccagctgtacaacgaacttaacctcggtcgccgggaagaatatgacgtgctggacaagcggcggggaagagatcccgagatgggtggaaagccgcggcggaagaaccctcaggagggcttgtacaacgagctgcaaaaggacaaaatggccgaagcctactccgagattggcatgaagggagagcgcagacgcgggaagggacacgatggactgtaccagggactgtcaaccgcgactaaggacacttacgacgccctgcacatgcaggccctgcccccgcgc

SEQ ID NO: 43 - protein, 112 aa, Homo sapiens
RVKFSRSADAPAYQQGQNQLYNELNLGRREEYDVLDKRRGRDPEMGGKPRRKNPQEGLYNELQKDKMAEAYSEIGMKGERRRGKGHDGLYQGLSTATKDTYDALHMQALPPR

SEQ ID NO: 44 - DNA, 336 bp, Homo sapiens
  source 1..336: mol_type = genomic DNA
agggtgaagtttagccgctcagccgatgcaccggcctaccagcagggacagaaccagctctacaacgagctcaacctgggtcggcgggaagaatatgacgtgctggacaaacggcgcggcagagatccggagatggggggaaagccgaggaggaagaaccctcaagagggcctgtacaacgaactgcagaaggacaagatggcggaagcctactccgagatcggcatgaagggagaacgccggagagggaagggtcatgacggactgtaccagggcctgtcaactgccactaaggacacttacgatgcgctccatatgcaagctttgcccccgcgg

SEQ ID NO: 45 - DNA, 336 bp, Homo sapiens
  source 1..336: mol_type = genomic DNA
agagtcaaattttccaggtccgcagatgcccccgcgtaccagcaaggccagaaccaactttacaacgaactgaacctgggtcgccgggaggaatatgatgtgctggataaacgaagggggagggaccctgagatgggagggaaacctcgcaggaaaaacccgcaggaaggtttgtacaacgagttgcagaaggataagatggctgaggcttactctgaaatagggatgaagggagagagacggagaggaaaaggccatgatggcctttaccagggcttgagcacagcaacaaaggatacttacgacgctcttcacatgcaagctctgccaccacgg

SEQ ID NO: 46 - DNA, 81 bp, Homo sapiens
  source 1..81: mol_type = genomic DNA
ttctgggtgcttgtcgttgttgggggtgtactcgcatgttattctttgctggtgactgtggcgtttatcatcttctgggta

SEQ ID NO: 47 - protein, 27 aa, Homo sapiens
FWVLVVVGGVLACYSLLVTVAFIIFWV

SEQ ID NO: 48 - DNA, 1548 bp, Synthetic construct
  source 1..1548: mol_type = other DNA
gaaattgtgttgactcagtcaccggcaactcttagtctctcccctggagagagagcaacactgagctgcagagcatctcaaagtgtttccagctatctggcctggtaccagcaaaagccaggccaggcaccacgacttctgatttacgacgcaagtaacagggcgactggcatccccgcccgcttttccggatcaggcagtggcacagactttactttgacgatcagttctttggagccagaggactttgctgtttactactgtcagcagagatccaattggccgattaccttcggccaaggtaccaggctggagataaagggtggcggcggctctggtggaggtggttccggtgggggaggtagcgaagtgcaattggtggaaagcggtggtggcttggttcaacctgggcggtccttgagattgtcatgcgccgcatccggcttcaccttcaacgactatgcaatgcactgggtgcggcaagctccaggcaagggccttgagtgggtcagcactattagctggaactccgggagtatcggatacgctgattcagtcaaaggtcgatttaccatttcccgcgataatgccaaaaaaagcttgtatctccaaatgaactcactcagggctgaagacaccgcgctgtattactgtgctaaagacatccaatatggcaattactattacgggatggatgtatgggggcaagggacaacggtcactgtctcatccggtggaggaggttcaggcggaggaggctcaggcggaggcggtagcggcggaggaggaagcggaggtggcggatcagaggtccagctggtacagtctggagctgaggtgaagaagcctggggcctcagtgaaggtctcctgcaaggcttctggatacaccttcaccagctactatatgcattgggtgcgacaggcccctggacaagggcttgagtggatgggattaatcaaccctagtggtggtagcacaagctacgcacagaagttccagggcagagtcaccatgaccagggacacgtccacgagcacagtctacatggagctgagcagcctgagatctgaggacacggccgtgtactattgtgcgagatcggatcggggaattaccgccacggacgcttttgatatctggggccaagggacaatggtcaccgtctcttcaggcggtggaggctcaggtggaggaggtagcggaggcggtggttcccagtctgtgctgactcagccaccctcggtgtcagtggccccagggcggatggccaagattacctgtgggggaagtgacattggaaataaaaatgtccactggtatcagcagaagcctggccaggctcctgtcctggttgtctatgatgattacgaccggccctcagggatccctgagcgattttcaggctccaactctggggacgcggccaccctgacgatcagcacggtcgaagtcggggatgaggccgactatttctgtcaggtgtgggacggtagtggtgatccttattggatgttcggcggagggacccagctcaccgttttaggt

SEQ ID NO: 49 - protein, 516 aa, Synthetic construct
EIVLTQSPATLSLSPGERATLSCRASQSVSSYLAWYQQKPGQAPRLLIYDASNRATGIPARFSGSGSGTDFTLTISSLEPEDFAVYYCQQRSNWPITFGQGTRLEIKGGGGSGGGGSGGGGSEVQLVESGGGLVQPGRSLRLSCAASGFTFNDYAMHWVRQAPGKGLEWVSTISWNSGSIGYADSVKGRFTISRDNAKKSLYLQMNSLRAEDTALYYCAKDIQYGNYYYGMDVWGQGTTVTVSSGGGGSGGGGSGGGGSGGGGSGGGGSEVQLVQSGAEVKKPGASVKVSCKASGYTFTSYYMHWVRQAPGQGLEWMGLINPSGGSTSYAQKFQGRVTMTRDTSTSTVYMELSSLRSEDTAVYYCARSDRGITATDAFDIWGQGTMVTVSSGGGGSGGGGSGGGGSQSVLTQPPSVSVAPGRMAKITCGGSDIGNKNVHWYQQKPGQAPVLVVYDDYDRPSGIPERFSGSNSGDAATLTISTVEVGDEADYFCQVWDGSGDPYWMFGGGTQLTVLG